SEQUENCE LISTING (43 sequences)

SEQ ID NO: 1 - protein, 10 aa, synthetic construct
  REGION 1..10: note = Synthetic
GFNIKDYFLH

SEQ ID NO: 2 - protein, 17 aa, synthetic construct
  REGION 1..17: note = Synthetic
WINPDNGNTVYDPKFQG

SEQ ID NO: 3 - protein, 11 aa, synthetic construct
  REGION 1..11: note = Synthetic
DYTYEKAALDY

SEQ ID NO: 4 - protein, 11 aa, synthetic construct
  REGION 1..11: note = Synthetic
RASGNIYNYLA

SEQ ID NO: 5 - protein, 7 aa, synthetic construct
  REGION 1..7: note = Synthetic
DAKTLAD

SEQ ID NO: 6 - protein, 9 aa, synthetic construct
  REGION 1..9: note = Synthetic
QHFWSLPFT

SEQ ID NO: 7 - protein, 139 aa, synthetic construct
  REGION 1..139: note = Synthetic
MALPVTALLLPLALLLHAEVQLQQSGAELVRPGALVKLSCKTSGFNIKDYFLHWVRQRPDQGLEWIGWINPDNGNTVYDPKFQGTASLTADTSSNTVYLQLSGLTSEDTAVYFCTRRDYTYEKAALDYWGQGASVIVSS

SEQ ID NO: 8 - protein, 110 aa, synthetic construct
  REGION 1..110: note = Synthetic
AFQMSQSPASLSASVGETVTITCRASGNIYNYLAWYQQKQGKSPHLLVYDAKTLADGVPSRFSGSGSGTQYSLKISSLQTEDSGNYYCQHFWSLPFTFGSGTKLEIKAAA

SEQ ID NO: 9 - DNA, 792 bp, synthetic construct
  misc_feature 1..792: note = Synthetic
  source 1..792: mol_type = other DNA
atggccttaccagtgaccgccttgctcctgccgctggccttgctgctccacgccgaggttcagctgcagcagtctggggctgagcttgtgaggccaggggccttggtcaagttgtcctgcaaaacttctggcttcaacattaaagactactttttacactgggtgagacagaggcctgaccagggcctggagtggattggatggattaatcctgataatggtaatactgtttatgacccgaagtttcagggcacggccagtttaacagcagacacatcctccaacacagtctacttgcagctcagcggcctgacatctgaggacactgccgtctatttctgtactcggagggactatacttatgaaaaggctgctctggactactggggtcagggagcctcagtcatcgtctcctcaggtggaggtggatcaggtggaggtggatctggtggaggtggatctgccttccagatgtctcagtctccagcctccctatctgcatctgtgggagaaactgtcaccatcacatgtcgagcaagtggaaatatttacaattatttagcatggtatcagcagaaacagggaaaatctcctcacctcctggtctatgatgcaaaaaccttagcagatggtgtgccatcaaggttcagtggcagtggctcagggacacaatattctctcaagattagcagcctgcagactgaagattctgggaattattactgtcaacatttttggagtcttccattcacgttcggctcggggacaaagttggaaataaaagcggccgca

SEQ ID NO: 10 - protein, 537 aa, Homo sapiens
MSAPKLLSLGCIFFPLLLFQQARAQFPRQCATVEALRSGMCCPDLSPVSGPGTDRCGSSSGRGRCEAVTADSRPHSPQYPHDGRDDREVWPLRFFNRTCHCNGNFSGHNCGTCRPGWRGAACDQRVLIVRRNLLDLSKEEKNHFVRALDMAKRTTHPLFVIATRRSEEILGPDGNTPQFENISIYNYFVWTHYYSVKKTFLGVGQESFGEVDFSHEGPAFLTWHRYHLLRLEKDMQEMLQEPSFSLPYWNFATGKNVCDICTDDLMGSRSNFDSTLISPNSVFSQWRVVCDSLEDYDTLGTLCNSTEDGPIRRNPAGNVARPMVQRLPEPQDVAQCLEVGLFDTPPFYSNSTNSFRNTVEGYSDPTGKYDPAVRSLHNLAHLFLNGTGGQTHLSPNDPIFVLLHTFTDAVFDEWLRRYNADISTFPLENAPIGHNRQYNMVPFWPPVTNTEMFVTAPDNLGYTYEIQWPSREFSVPEIIAIAVVGALLLVALIFGTASYLIRARRSMDEANQPLLTDQYQCYAEEYEKLQNPNQSVV

SEQ ID NO: 11 - protein, 15 aa, synthetic construct
  REGION 1..15: note = Synthetic
GGGGSGGGGSGGGGS

SEQ ID NO: 12 - protein, 18 aa, synthetic construct
  REGION 1..18: note = Synthetic
MALPVTALLLPLALLLHA

SEQ ID NO: 13 - protein, 235 aa, Homo sapiens
MALPVTALLLPLALLLHAARPSQFRVSPLDRTWNLGETVELKCQVLLSNPTSGCSWLFQPRGAAASPTFLLYLSQNKPKAAEGLDTQRFSGKRLGDTFVLTLSDFRRENEGYYFCSALSNSIMYFSHFVPVFLPAKPTTTPAPRPPTPAPTIASQPLSLRPEACRPAAGGAVHTRGLDFACDIYIWAPLAGTCGVLLLSLVITLYCNHRNRRRVCKCPRPVVKSGDKPSLSARYV

SEQ ID NO: 14 - protein, 220 aa, Homo sapiens
MLRLLLALNLFPSIQVTGNKILVKQSPMLVAYDNAVNLSCKYSYNLFSREFRASLHKGLDSAVEVCVVYGNYSQQLQVYSKTGFNCDGKLGNESVTFYLQNLYVNQTDIYFCKIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRS

SEQ ID NO: 15 - protein, 164 aa, Homo sapiens
MKWKALFTAAILQAQLPITEAQSFGLLDPKLCYLLDGILFIYGVILTALFLRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 16 - protein, 188 aa, Mus musculus
MKWKVSVLACILHVRFPGAEAQSFGLLDPKLCYLLDGILFIYGVIITALYLRAKFSRSAETAANLQDPNQLYNELNLGRREEYDVLEKKRARDPEMGGKQRRRNPQEGVYNALQKDKMAEAYSEIGTKGERRRGKGHDGLYQDSHFQAVQFGNRREREGSELTRTLGLRARPKACRHKKPLSLPAAVS

SEQ ID NO: 17 - protein, 255 aa, Homo sapiens
MGNSCYNIVATLLLVLNFERTRSLQDPCSNCPAGTFCDNNRNQICSPCPPNSFSSAGGQRTCDICRQCKGVFRTRKECSSTSNAECDCTPGFHCLGAGCSMCEQDCKQGQELTKKGCKDCCFGTFNDQKRGICRPWTNCSLDGKSVLVNGTKERDVVCGPSPADLSPGASSVTPPAPAREPGHSPQIISFFLALTSTALLFLLFFLTLRFSVVKRGRKKLLYIFKQPFMRPVQTTQEEDGCSCRFPEEEEGGCEL

SEQ ID NO: 18 - protein, 277 aa, Homo sapiens
MCVGARRLGRGPCAALLLLGLGLSTVTGLHCVGDTYPSNDRCCHECRPGNGMVSRCSRSQNTVCRPCGPGFYNDVVSSKPCKPCTWCNLRSGSERKQLCTATQDTVCRCRAGTQPLDSYKPGVDCAPCPPGHFSPGDNQACKPWTNCTLAGKHTLQPASNSSDAICEDRDPPATQPQETQGPPARPITVQPTEAWPRTSQGPSTRPVEVPGGRAVAAILGLGLVLGLLGPLAILLALYLLRRDQRLPPDAHKPPGGGSFRTPIQEEQADAHSTLAKI

SEQ ID NO: 19 - protein, 199 aa, Homo sapiens
MKSGLWYFFLFCLRIKVLTGEINGSANYEMFIFHNGGVQILCKYPDIVQQFKMQLLKGGQILCDLTKTKGSGNTVSIKSLKFCHSQLSNNSVSFFLYNLDHSHANYYFCNLSIFDPPPFKVTLTGGYLHIYESQLCCQLKFWLPIGCAAFVVVCILGCILICWLTKKKYSSSVHDPNGEYMFMRAVNTAKKSRLTDVTL

SEQ ID NO: 20 - protein, 223 aa, Homo sapiens
MACLGFQRHKAQLNLATRTWPCTLLFFLLFIPVFCKAMHVAQPAVVLASSRGIASFVCEYASPGKATEVRVTVLRQADSQVTEVCAATYMMGNELTFLDDSICTGTSSGNQVNLTIQGLRAMDTGLYICKVELMYPPPYYLGIGNGTQIYVIDPEPCPDSDFLLWILAAVSSGLFFYSFLLTAVSLSKMLKKRSPLTTGVYVKMPPTEPECEKQFQPYFIPIN

SEQ ID NO: 21 - protein, 288 aa, Homo sapiens
MQIPQAPWPVVWAVLQLGWRPGWFLDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPAGQFQTLVVGVVGGLLGSLVLLVWVLAVICSRAARGTIGARRTGQPLKEDPSAVPVFSVDYGELDFQWREKTPEPPVPCVPEQTEYATIVFPSGMGTSSPARRGSADGPRSAQPLRPEDGHCSWPL

SEQ ID NO: 22 - protein, 525 aa, Homo sapiens
MWEAQFLGLLFLQPLWVAPVKPLQPGAEVPVVWAQEGAPAQLPCSPTIPLQDLSLLRRAGVTWQHQPDSGPPAAAPGHPLAPGPHPAAPSSWGPRPRRYTVLSVGPGGLRSGRLPLQPRVQLDERGRQRGDFSLWLRPARRADAGEYRAAVHLRDRALSCRLRLRLGQASMTASPPGSLRASDWVILNCSFSRPDRPASVHWFRNRGQGRVPVRESPHHHLAESFLFLPQVSPMDSGPWGCILTYRDGFNVSIMYNLTVLGLEPPTPLTVYAGAGSRVGLPCRLPAGVGTRSFLTAKWTPPGGGPDLLVTGDNGDFTLRLEDVSQAQAGTYTCHIHLQEQQLNATVTLAIITVTPKSFGSPGSLGKLLCEVTPVSGQERFVWSSLDTPSQRSFSGPWLEAQEAQLLSQPWQCQLYQGERLLGAAVYFTELSSPGAQRSGRAPGALPAGHLLLFLILGVLSLLLLVTGAFGFHLWRRQWRPRRFSALEQGIHPPQAQSKIEELEQEPEPEPEPEPEPEPEPEPEQL

SEQ ID NO: 23 - protein, 370 aa, Homo sapiens
MLGQVVTLILLLLLKVYQGKGCQGSADHVVSISGVPLQLQPNSIQTKVDSIAWKKLLPSQNGFHHILKWENGSLPSNTSNDRFSFIVKNLSLLIKAAQQQDSGLYCLEVTSISGKVQTATFQVFVFESLLPDKVEKPRLQGQGKILDRGRCQVALSCLVSRDGNVSYAWYRGSKLIQTAGNLTYLDEEVDINGTHTYTCNVSNPVSWESHTLNLTQDCQNAHQEFRFWPFLVIIVILSALFLGTLACFCVWRRKRKEKQSETSPKEFLTIYEDVKDLKTRRNHEQEQTFPGGGSTIYSMIQSQSSAPTSQEPAYTLYSLIQPSRKSGSRKRNHSPSFNSTIYEVIGKSQPKAQNPARLSRKELENFDVYS

SEQ ID NO: 24 - protein, 289 aa, Homo sapiens
MKTLPAMLGTGKLFWVFFLIPYLDIWNIHGKESCDVQLYIKRQSEHSILAGDPFELECPVKYCANRPHVTWCKLNGTTCVKLEDRQTSWKEEKNISFFILHFEPVLPNDNGSYRCSANFQSNLIESHSTTLYVTDVKSASERPSKDEMASRPWLLYRLLPLGGLPLLITTCFCLFCCLRRHQGKQNELSDTAGREINLVDAHLKSEQTEASTRQNSQVLLSETGIYDNDPDLCFRMQEGSEVYSNPCLEENKPGIVYASLNHSVIGPNSRLARNVKEAPTEYASICVRS

SEQ ID NO: 25 - DNA, 1464 bp, synthetic construct
  misc_feature 1..1464: note = Synthetic
  source 1..1464: mol_type = other DNA
atggccttaccagtgaccgccttgctcctgccgctggccttgctgctccacgccgaggttcagctgcagcagtctggggctgagcttgtgaggccaggggccttggtcaagttgtcctgcaaaacttctggcttcaacattaaagactactttttacactgggtgagacagaggcctgaccagggcctggagtggattggatggattaatcctgataatggtaatactgtttatgacccgaagtttcagggcacggccagtttaacagcagacacatcctccaacacagtctacttgcagctcagcggcctgacatctgaggacactgccgtctatttctgtactcggagggactatacttatgaaaaggctgctctggactactggggtcagggagcctcagtcatcgtctcctcaggtggaggtggatcaggtggaggtggatctggtggaggtggatctgccttccagatgtctcagtctccagcctccctatctgcatctgtgggagaaactgtcaccatcacatgtcgagcaagtggaaatatttacaattatttagcatggtatcagcagaaacagggaaaatctcctcacctcctggtctatgatgcaaaaaccttagcagatggtgtgccatcaaggttcagtggcagtggctcagggacacaatattctctcaagattagcagcctgcagactgaagattctgggaattattactgtcaacatttttggagtcttccattcacgttcggctcggggacaaagttggaaataaaagcggccgcatctactactaccaagccagtgctgcgaactccctcacctgtgcaccctaccgggacatctcagccccagagaccagaagattgtcggccccgtggctcagtgaaggggaccggattggacttcgcctgtgatatttacatctgggcacccttggccggaatctgcgtggcccttctgctgtccttgatcatcactctcatctgctacaatagtagaaggaacagactccttcaaagtgactacatgaacatgactccccggaggcctgggctcactcgaaagccttaccagccctacgcccctgccagagactttgcagcgtaccgccccagagcaaaattcagcaggagtgcagagactgctgccaacctgcaggaccccaaccagctctacaatgagctcaatctagggcgaagagaggaatatgacgtcttggagaagaagcgggctcgggatccagagatgggaggcaaacagcagaggaggaggaacccccaggaaggcgtatacaatgcactgcagaaagacaagatggcagaagcctacagtgagatcggcacaaaaggcgagaggcggagaggcaaggggcacgatggcctttaccagggtctcagcactgccaccaaggacacctatgatgccctgcatatgcagaccctggcccctcgctaa

SEQ ID NO: 26 - DNA, 45 bp, synthetic construct
  misc_feature 1..45: note = Synthetic
  source 1..45: mol_type = other DNA
ggtggaggtggatcaggtggaggtggatctggtggaggtggatct

SEQ ID NO: 27 - DNA, 417 bp, synthetic construct
  misc_feature 1..417: note = Synthetic
  source 1..417: mol_type = other DNA
atggccttaccagtgaccgccttgctcctgccgctggccttgctgctccacgccgaggttcagctgcagcagtctggggctgagcttgtgaggccaggggccttggtcaagttgtcctgcaaaacttctggcttcaacattaaagactactttttacactgggtgagacagaggcctgaccagggcctggagtggattggatggattaatcctgataatggtaatactgtttatgacccgaagtttcagggcacggccagtttaacagcagacacatcctccaacacagtctacttgcagctcagcggcctgacatctgaggacactgccgtctatttctgtactcggagggactatacttatgaaaaggctgctctggactactggggtcagggagcctcagtcatcgtctcctca

SEQ ID NO: 28 - DNA, 330 bp, synthetic construct
  misc_feature 1..330: note = Synthetic
  source 1..330: mol_type = other DNA
gccttccagatgtctcagtctccagcctccctatctgcatctgtgggagaaactgtcaccatcacatgtcgagcaagtggaaatatttacaattatttagcatggtatcagcagaaacagggaaaatctcctcacctcctggtctatgatgcaaaaaccttagcagatggtgtgccatcaaggttcagtggcagtggctcagggacacaatattctctcaagattagcagcctgcagactgaagattctgggaattattactgtcaacatttttggagtcttccattcacgttcggctcggggacaaagttggaaataaaagcggccgca

SEQ ID NO: 29 - DNA, 54 bp, synthetic construct
  misc_feature 1..54: note = Synthetic
  source 1..54: mol_type = other DNA
atggccttaccagtgaccgccttgctcctgccgctggccttgctgctccacgcc

SEQ ID NO: 30 - protein, 247 aa, Mus musculus
MASPLTRFLSLNLLLMGESIILGSGEAKPQAPELRIFPKKMDAELGQKVDLVCEVLGSVSQGCSWLFQNSSSKLPQPTFVVYMASSHNKITWDEKLNSSKLFSAVRDTNNKYVLTLNKFSKENEGYYFCSVISNSVMYFSSVVPVLQKVNSTTTKPVLRTPSPVHPTGTSQPQRPEDCRPRGSVKGTGLDFACDIYIWAPLAGICVAPLLSLIITLICYHRSRKRVCKCPRPLVRQEGKPRPSEKIV

SEQ ID NO: 31 - DNA, 342 bp, Homo sapiens
  source 1..342: mol_type = other DNA
agagcaaaattcagcaggagtgcagagactgctgccaacctgcaggaccccaaccagctctacaatgagctcaatctagggcgaagagaggaatatgacgtcttggagaagaagcgggctcgggatccagagatgggaggcaaacagcagaggaggaggaacccccaggaaggcgtatacaatgcactgcagaaagacaagatggcagaagcctacagtgagatcggcacaaaaggcgagaggcggagaggcaaggggcacgatggcctttaccagggtctcagcactgccaccaaggacacctatgatgccctgcatatgcagaccctggcccctcgctaa

SEQ ID NO: 32 - protein, 69 aa, Homo sapiens
STTTKPVLRTPSPVHPTGTSQPQRPEDCRPRGSVKGTGLDFACDIYIWAPLAGICVALLLSLIITLICY

SEQ ID NO: 33 - DNA, 207 bp, Homo sapiens
  source 1..207: mol_type = other DNA
tctactactaccaagccagtgctgcgaactccctcacctgtgcaccctaccgggacatctcagccccagagaccagaagattgtcggccccgtggctcagtgaaggggaccggattggacttcgcctgtgatatttacatctgggcacccttggccggaatctgcgtggcccttctgctgtccttgatcatcactctcatctgctac

SEQ ID NO: 34 - protein, 113 aa, Homo sapiens
RAKFSRSAETAANLQDPNQLYNELNLGRREEYDVLEKKRARDPEMGGKQQRRRNPQEGVYNALQKDKMAEAYSEIGTKGERRRGKGHDGLYQGLSTATKDTYDALHMQTLAPR

SEQ ID NO: 35 - protein, 218 aa, Homo sapiens
MTLRLLFLALNFFSVQVTENKILVKQSPLLVVDSNEVSLSCRYSYNLLAKEFRASLYKGVNSDVEVCVGNGNFTYQPQFRSNAEFNCDGDFDNETVTFRLWNLHVNHTDIYFCKIEFMYPPPYLDNERSNGTIIHIKEKHLCHTQSSPKLFWALVVVAGVLFCYGLLVTVALCVIWTNSRRNRLLQSDYMNMTPRRPGLTRKPYQPYAPARDFAAYRP

SEQ ID NO: 36 - DNA, 96 bp, Homo sapiens
  source 1..96: mol_type = other DNA
agtgactacatgaacatgactccccggaggcctgggctcactcgaaagccttaccagccctacgcccctgccagagactttgcagcgtaccgcccc

SEQ ID NO: 37 - protein, 537 aa, Mus musculus
MKSYNVLPLAYISLFLMLFYQVWAQFPRECANIEALRRGVCCPDLLPSSGPGTDPCGSSSGRGRCVAVIADSRPHSRHYPHDGKDDREAWPLRFFNRTCQCNDNFSGHNCGTCRPGWRGAACNQKILTVRRNLLDLSPEEKSHFVRALDMAKRTTHPQFVIATRRLEDILGPDGNTPQFENISVYNYFVWTHYYSVKKTFLGTGQESFGDVDFSHEGPAFLTWHRYHLLQLERDMQEMLQEPSFSLPYWNFATGKNVCDVCTDDLMGSRSNFDSTLISPNSVFSQWRVVCESLEEYDTLGTLCNSTEGGPIRRNPAGNVGRPAVQRLPEPQDVTQCLEVRVFDTPPFYSNSTDSFRNTVEGYSAPTGKYDPAVRSLHNLAHLFLNGTGGQTHLSPNDPIFVLLHTFTDAVFDEWLRRYNADISTFPLENAPIGHNRQYNMVPFWPPVTNTEMFVTAPDNLGYAYEVQWPGQEFTVSEIITIAVVAALLLVAAIFGVASCLIRSRSTKNEANQPLLTDHYQRYAEDYEELPNPNHSMV

SEQ ID NO: 38 - DNA, 30 bp, synthetic construct
  misc_feature 1..30: note = Synthetic
  source 1..30: mol_type = other DNA
ggcttcaacattaaagactactttttacac

SEQ ID NO: 39 - DNA, 51 bp, synthetic construct
  misc_feature 1..51: note = Synthetic
  source 1..51: mol_type = other DNA
tggattaatcctgataatggtaatactgtttatgacccgaagtttcagggc

SEQ ID NO: 40 - DNA, 33 bp, synthetic construct
  misc_feature 1..33: note = Synthetic
  source 1..33: mol_type = other DNA
gactatacttatgaaaaggctgctctggactac

SEQ ID NO: 41 - DNA, 33 bp, synthetic construct
  misc_feature 1..33: note = Synthetic
  source 1..33: mol_type = other DNA
cgagcaagtggaaatatttacaattatttagca

SEQ ID NO: 42 - DNA, 21 bp, synthetic construct
  misc_feature 1..21: note = Synthetic
  source 1..21: mol_type = other DNA
gatgcaaaaaccttagcagat

SEQ ID NO: 43 - DNA, 27 bp, synthetic construct
  misc_feature 1..27: note = Synthetic
  source 1..27: mol_type = other DNA
caacatttttggagtcttccattcacg

(19) United States
(12) Patent Application Publication
(10) Pub. No.: US 2025/0026855 A1
(43) Pub. Date: Jan. 23, 2025

(54) ANTIGEN-BINDING PROTEINS TARGETING MELANOMA DIFFERENTIATION ANTIGENS AND USES THEREOF

(71) Applicant: MEMORIAL SLOAN-KETTERING CANCER CENTER, New York, NY (US)

(72) Inventors: Taha Merghoub, Jerssey City, NJ (US); Jedd D. Wolchok, New York, NY (US); Daniel Hirschhorn, New York, NY (US); Cailian Liu, Forest Hills, NY (US)

(73) Assignee: MEMORIAL SLOAN-KETTERING CANCER CENTER, New York, NY (US)

(21) Appl. No.: 18/506,518

(22) Filed: Nov. 10, 2023

Related U.S. Application Data
(63) Continuation of application No. 16/388,245, filed on Apr. 18, 2019, now abandoned.
Application No. 16/388,245 is a continuation of application No. PCT/US17/57098, filed on Oct. 18, 2017.
(60) Provisional application No. 62/409,577, filed on Oct. 18, 2016.

(51) Int. Cl. C07K 16/40; A61K 38/00; A61K 39/00; A61P 35/00; C07K 14/705; C07K 14/725; C12N 5/0783

(52) U.S. Cl. CPC C07K 16/40; A61K 39/4611; A61K 39/4613; A61K 39/4631; A61K 39/4632; A61K 39/464456; A61K 39/46449; A61K 2239/31; A61K 2239/38; A61K 2239/57; A61P 35/00; C07K 14/7051; C07K 14/70517; C07K 14/70521; C12N 5/0636; A61K 38/00; A61K 2039/505; C07K 2317/33; C07K 2317/565; C07K 2317/622; C07K 2317/92; C07K 2319/02; C07K 2319/03; C07K 2319/30; C07K 2319/33

(57) ABSTRACT

The presently disclosed subject matter provides methods and compositions for treating cancer (e.g., melanoma). It relates to chimeric antigen receptors (CARs) that specifically target MDA (e.g., Trp1), and immunoresponsive cells comprising such CARs. The presently disclosed MDA-specific CARs have enhanced immune-activating properties, including anti-tumor activity.

### CROSS-REFERENCE TO RELATED APPLICATION

This application is a Continuation of U.S. patent application Ser. No. 16/388,245, filed on Apr. 18, 2019, which is a Continuation of International Patent Application No. PCT/US2017/057098, filed Oct. 18, 2017, which claims priority to U.S. Provisional Application No. 62/409,577 filed on Oct. 18, 2016, the contents of each of which are incorporated by reference in their entireties, and to each of which priority is claimed.

### GRANT INFORMATION

This invention was made with government support under CA056821 awarded by the National Institutes of Health. The government has certain rights in the invention.

### SEQUENCE LISTING

The specification further incorporates by reference the Sequence Listing submitted herewith via on Nov. 10, 2023. The Sequence Listing text file, identified as 0727340867CON.xml, is 57,344 bytes and was created on Nov. 10, 2023. The Sequence Listing, electronically filed herewith, does not extend beyond the scope of the specification and thus does not contain new matter.

### INTRODUCTION

The presently disclosed subject matter provides methods and compositions for treating cancer. It relates to antigen-binding proteins that include antibodies, or antigen-binding portions thereof, and chimeric antigen receptors (CARs) that specifically target melanoma differentiation antigens (MDA). The presently disclosed subject matter further includes immunoresponsive cells comprising such CARs, and methods of using such cells for treating cancers (e.g., melanoma).

### BACKGROUND OF THE SUBJECT MATTER

Cell-based immunotherapy is a therapy with curative potential for the treatment of cancer. T cells and other immune cells may be modified to target tumor antigens through the introduction of genetic material coding for artificial or synthetic receptors for antigen, termed Chimeric Antigen Receptors (CARs), specific to selected antigens. Targeted T cell therapy using CARs has shown recent clinical success in treating hematologic malignancies.

Malignant melanoma is the deadliest skin cancer and is refractory to conventional therapies. Recent clinical studies have shown that potentiating the immune system with monoclonal antibodies (mAbs) can be successful in treating metastatic melanoma (Sharma, et al., Nat Rev Cancer, 11:805-12, 2011). Certain antigens represent molecules associated with the melanocyte lineage and are called melanoma differentiation antigens (MDA) (Hearing, et al, Pigment Cell Res 5: 264-270, 1992). Recent progress with melanoma vaccines has indicated that T cell immunity against MDA can be induced in patients of advanced melanoma (Collela, et al., J Exp Med 191: 1221-1232, 2000). However, inducing immune responses against MDA remains challenging due to unclear reasons. Further, advanced tumors acquire various immune-escape mechanisms that prevent full T cell activation, which hampers traditional immune therapy (Dunn, et al., Nat Immunol 3: 991-998, 2002). Thus, more effective therapy is needed for melanoma treatment.

There has been emerging interest in cellular immunotherapy using T cells expressing either T cell receptors (TCRs) or CARs targeted against melanoma associated antigens following the successful use of CD19 targeted CARs in patients with chronic lymphocytic leukemia and acute lymphoblastic leukemia (Brentjens, R. J., et al. Eradication of systemic B-cell tumors by genetically targeted human T lymphocytes co-stimulated by CD80 and interleukin-15. Nature medicine 9, 279-286 (2003); Brentjens, R. J., et al. CD19-Targeted T Cells Rapidly Induce Molecular Remissions in Adults with Chemotherapy-Refractory Acute Lymphoblastic Leukemia. Science translational medicine 5, 177ra138 (2013); Porter, et al. Chimeric antigen receptor-modified T cells in chronic lymphoid leukemia. N. Engl. J. Med. 365:725-733 (2011)). While there are various reasons to expect that adoptive T cell therapy may work well in melanoma, expanding adoptive T cell therapy to melanoma also poses unique challenges. Accordingly, there is a need for novel therapeutic strategies capable of inducing potent tumor eradication with minimal toxicity and immunogenicity.

### SUMMARY OF THE SUBJECT MATTER

The presently disclosed subject matter generally provides chimeric antigen receptors (CARs) that specifically target an MDA (e.g., Tyrosinase-related protein 1 (Trp1)), immunoresponsive cells comprising such CARs, and uses of these CARs and immunoresponsive cells for treating cancers (e.g., melanoma).

In certain embodiments, the CAR comprises an extracellular antigen-binding domain, a transmembrane domain and an intracellular domain, wherein the extracellular antigen-binding domain specifically binds to an MDA polypeptide. In certain embodiments, the MDA polypeptide is selected from the group consisting of TRP1, tyrosinase, Melan-A, gp100, and TRP2. In certain embodiments, the MDA polypeptide is a Trp1 polypeptide. In certain embodiments, the extracellular antigen-binding domain cross-reacts with a mouse Trp1 polypeptide and a human Trp1 polypeptide. In certain embodiments, the extracellular antigen-binding domain specifically binds to an MDA polypeptide with a binding affinity (K_{d}) of about 3×10⁻⁹ M or less.

In certain embodiments, the extracellular antigen-binding domain comprises a heavy chain variable region comprising an amino acid sequence that is at least about 80% homologous to the sequence set forth in SEQ ID NO:7. In certain embodiments, the extracellular antigen-binding domain comprises a heavy chain variable region comprising amino acids having the sequence set forth in SEQ ID NO:7.

In certain embodiments, the extracellular antigen-binding domain comprises a light chain variable region comprising an amino acid sequence that is at least about 80% homologous to the sequence set forth in SEQ ID NO:8. In certain embodiments, wherein the extracellular antigen-binding domain comprises a light chain variable region comprising amino acids having the sequence set forth in SEQ ID NO:8.

In certain embodiments, the extracellular antigen-binding domain comprises a heavy chain variable region comprising an amino acid sequence that is at least about 80% homologous to the sequence set forth in SEQ ID NO:7, and a light chain variable region comprising an amino acid sequence that is at least about 80% homologous to the sequence set forth in SEQ ID NO:8. In certain embodiments, the extracellular antigen-binding domain comprises a heavy chain variable region comprising amino acids having the sequence set forth in SEQ ID NO:7, and a light chain variable region comprising amino acids having the sequence set forth in SEQ ID NO:8.

In certain embodiments, the extracellular antigen-binding domain comprises a heavy chain variable region CDR1 comprising amino acids having the sequence set forth in SEQ ID NO:1 or a conservative modification thereof; a heavy chain variable region CDR2 comprising amino acids having the sequence set forth in SEQ ID NO: 2 or a conservative modification thereof; and a heavy chain variable region CDR3 comprising amino acids having the sequence set forth in SEQ ID NO:3 or a conservative modification thereof. In certain embodiments, the extracellular antigen-binding domain comprises a heavy chain variable region CDR1 comprising amino acids having the sequence set forth in SEQ ID NO:1; a heavy chain variable region CDR2 comprising amino acids having the sequence set forth in SEQ ID NO: 2; and a heavy chain variable region CDR3 comprising amino acids having the sequence set forth in SEQ ID NO:3.

In certain embodiments, the extracellular antigen-binding domain comprises a light chain variable region CDR1 comprising amino acids having the sequence set forth in SEQ ID NO:4 or a conservative modification thereof; a light chain variable region CDR2 comprising amino acids having the sequence set forth in SEQ ID NO:5 or a conservative modification thereof; and a light chain variable region CDR3 comprising amino acids having the sequence set forth in SEQ ID NO:6 or a conservative modification thereof. In certain embodiments, the extracellular antigen-binding domain comprises a light chain variable region CDR1 comprising amino acids having the sequence set forth in SEQ ID NO:4; a light chain variable region CDR2 comprising amino acids having the sequence set forth in SEQ ID NO:5; and a light chain variable region CDR3 comprising amino acids having the sequence set forth in SEQ ID NO:6.

In certain embodiments, the extracellular antigen-binding domain comprises a heavy chain variable region CDR3 comprising amino acids having the sequence set forth in SEQ ID NO:3 or a conservative modification thereof, and a light chain variable region CDR3 comprising amino acids having the sequence set forth in SEQ ID NO:6 or a conservative modification thereof. In certain embodiments, the extracellular antigen-binding domain comprises a heavy chain variable region CDR2 comprising amino acids having the sequence set forth in SEQ ID NO: 2 or a conservative modification thereof, and a light chain variable region CDR2 comprising amino acids having the sequence set forth in SEQ ID NO: 5 or a conservative modification thereof. In certain embodiments, the extracellular antigen-binding domain comprises a heavy chain variable region CDR1 comprising amino acids having the sequence set forth in SEQ ID NO: 1 or a conservative modification thereof, and a light chain variable region CDR1 comprising amino acids having the sequence set forth in SEQ ID NO: 4.

In certain embodiments, the extracellular antigen-binding domain comprises a heavy chain variable region CDR1 comprising amino acids having the sequence set forth in SEQ ID NO: 1 or a conservative modification thereof; a heavy chain variable region CDR2 comprising amino acids having the sequence set forth in SEQ ID NO: 2 or a conservative modification thereof; a heavy chain variable region CDR3 comprising amino acids having the sequence set forth in SEQ ID NO: 3 or a conservative modification thereof; a light chain variable region CDR1 comprising amino acids having the sequence set forth in SEQ ID NO: 4 or a conservative modification thereof; a light chain variable region CDR2 comprising amino acids having the sequence set forth in SEQ ID NO: 5 or a conservative modification thereof; and a light chain variable region CDR3 comprising amino acids having the sequence set forth in SEQ ID NO: 6 or a conservative modification thereof. In certain embodiments, the extracellular antigen-binding domain comprises a heavy chain variable region CDR1 comprising amino acids having the sequence set forth in SEQ ID NO: 1; a heavy chain variable region CDR2 comprising amino acids having the sequence set forth in SEQ ID NO: 2; a heavy chain variable region CDR3 comprising amino acids having the sequence set forth in SEQ ID NO: 3; a light chain variable region CDR1 comprising amino acids having the sequence set forth in SEQ ID NO: 4; a light chain variable region CDR2 comprising amino acids having the sequence set forth in SEQ ID NO: 5; and a light chain variable region CDR3 comprising amino acids having the sequence set forth in SEQ ID NO: 6.

The presently disclosed subject matter further provides CARs comprising an extracellular antigen-binding domain, a transmembrane domain and an intracellular domain, wherein the extracellular antigen-binding domain cross-competes for binding to an MDA polypeptide with a reference antibody or an antigen-binding portion thereof comprising a heavy chain variable region CDR1 comprising amino acids having the sequence set forth in SEQ ID NO: 1; a heavy chain variable region CDR2 comprising amino acids having the sequence set forth in SEQ ID NO: 2; a heavy chain variable region CDR3 comprising amino acids having the sequence set forth in SEQ ID NO: 3; a light chain variable region CDR1 comprising amino acids having the sequence set forth in SEQ ID NO: 4; a light chain variable region CDR2 comprising amino acids having the sequence set forth in SEQ ID NO:5; and a light chain variable region CDR3 comprising amino acids having the sequence set forth in SEQ ID NO:6.

The presently disclosed subject matter also provides CARs comprising an extracellular antigen-binding domain, a transmembrane domain and an intracellular domain, wherein the extracellular antigen-binding domain binds to the same epitope on an MDA polypeptide as a reference antibody or an antigen-binding portion thereof comprising a heavy chain variable region CDR1 comprising amino acids having the sequence set forth in SEQ ID NO:1; a heavy chain variable region CDR2 comprising amino acids having the sequence set forth in SEQ ID NO:2; a heavy chain variable region CDR3 comprising amino acids having the sequence set forth in SEQ ID NO: 3; a light chain variable region CDR1 comprising amino acids having the sequence set forth in SEQ ID NO: 4; a light chain variable region CDR2 comprising amino acids having the sequence set forth in SEQ ID NO:5; and a light chain variable region CDR3 comprising amino acids having the sequence set forth in SEQ ID NO:6.

In certain embodiments, the reference antibody or antigen-binding portion thereof comprises a heavy chain variable region comprising amino acids having the sequence set forth in SEQ ID NO: 7, and a light chain variable region comprising amino acids having the sequence set forth in SEQ ID NO:8.

In certain non-limiting embodiments, the extracellular antigen-binding domain comprises both of the heavy and light chain variable regions, optionally with a linker sequence, for example a linker peptide, between the heavy chain variable region and the light chain variable region. In certain embodiments, the extracellular antigen-binding domain is a single-chain variable fragment (scFv). In certain embodiments, the extracellular antigen-binding domain is a murine scFv. In certain embodiments, the extracellular antigen-binding domain is a Fab, which is optionally crosslinked. In certain embodiments, the extracellular binding domain is a F(ab)₂. In certain embodiments, any of the foregoing molecules can be comprised in a fusion protein with a heterologous sequence to form the extracellular antigen-binding domain.

In accordance with the presently disclosed subject matter, the extracellular antigen-binding domain is covalently joined to a transmembrane domain. The extracellular antigen-binding domain can comprise a signal peptide that is covalently joined to the 5′ terminus of the extracellular antigen-binding domain. In certain embodiments, the transmembrane domain of a presently disclosed CAR comprises a CD8 polypeptide, a CD28 polypeptide, a CD3zeta polypeptide, a CD4 polypeptide, a 4-1BB polypeptide, an OX40 polypeptide, an ICOS polypeptide, a CTLA-4 polypeptide, a PD-1 polypeptide, a LAG-3 polypeptide, a 2B4 polypeptide, a BTLA polypeptide, a synthetic peptide (not based on a protein associated with the immune response), or a combination thereof. In certain embodiments, the transmembrane domain comprises a CD8 polypeptide.

In accordance with the presently disclosed subject matter, the intracellular domain comprises a CD3zeta polypeptide. In certain embodiments, the intracellular domain further comprises at least one signaling region. In certain embodiments, the at least one signaling region comprises a CD28 polypeptide, a 4-1BB polypeptide, an OX40 polypeptide, an ICOS polypeptide, a DAP-10 polypeptide, a PD-1 polypeptide, a CTLA-4 polypeptide, a LAG-3 polypeptide, a 2B4 polypeptide, a BTLA polypeptide, a synthetic peptide (not based on a protein associated with the immune response), or a combination thereof. In certain embodiments, the signaling region is a co-stimulatory signaling region. In certain embodiments, the co-stimulatory signaling region comprises a CD28 polypeptide, a 4-1BB polypeptide, an OX40 polypeptide, an ICOS polypeptide, a DAP-10 polypeptide, or a combination thereof. In certain embodiments, the at least one co-stimulatory signaling region comprises a CD28 polypeptide. In certain embodiments, the CAR comprises a transmembrane domain that comprises a CD8 polypeptide, an intracellular domain that comprises a CD3zeta polypeptide and a co-stimulatory signaling region that comprises a CD28 polypeptide.

In certain embodiments, the CAR is recombinantly expressed. The CAR can be expressed from a vector. In certain embodiments, the vector is a γ-retroviral vector. The presently disclosed subject matter also provides isolated immunoresponsive cells comprising the above-described CARs. In certain embodiments, the isolated immunoresponsive cell is transduced with the CAR, for example, the CAR is constitutively expressed on the surface of the immunoresponsive cell. In certain embodiments, the isolated immunoresponsive cell is selected from the group consisting of a T cell, a Natural Killer (NK) cell, a human embryonic stem cell, a lymphoid progenitor cell, a T cell-precursor cell, and a pluripotent stem cell from which lymphoid cells may be differentiated. In certain embodiments, the immunoresponsive cell is a T cell. In certain embodiments, the T cell is selected from the group consisting of a cytotoxic T lymphocyte (CTL), a regulatory T cell, a helper T cell, an NK T cell and central memory T cells.

The presently disclosed subject matter further provides nucleic acid molecules encoding the presently disclosed CARs, vectors comprising the nucleic acid molecules, and host cells expressing such nucleic acid molecules. In certain embodiments, the vector is a γ-retroviral vector. In certain embodiments, the host cell is a T cell.

The presently disclosed subject matter further provides pharmaceutical compositions comprising an effective amount of a presently disclosed CAR or a presently disclosed immunoresponsive cell and a pharmaceutically acceptable excipient. In certain embodiments, the pharmaceutical compositions are for treating a neoplasia.

Furthermore, the presently disclosed subject matter provides methods of using a CAR, an immunoresponsive cell, or a pharmaceutical composition disclosed herein for reducing tumor burden in a subject. For example, and not by way of limitation, the presently disclosed subject matter provides methods of reducing tumor burden in a subject, wherein the method comprises administering an effective amount of a presently disclosed CAR or a presently disclosed immunoresponsive cell to the subject, thereby inducing tumor cell death in the subject. In certain embodiments, the subject receives a chemotherapeutic agent. A chemotherapeutic agent can be used as lymphoablative conditioning regimen. In certain embodiments, the chemotherapeutic agent is selected from the group consisting of docetaxel, cyclophosphamide, capecitabine, doxorubic, fludarabin, and combinations thereof. In certain embodiments, the chemotherapeutic agent is cyclophosphamide. In certain embodiments, the subject receives the chemotherapeutic agent prior to the CAR, immunoresponsive cell, pharmaceutical composition. In certain embodiments, the method reduces the number of tumor cells. In certain embodiments, the method reduces the tumor size. In certain embodiments, the method eradicates the tumor in the subject. In certain embodiments, the tumor is associated with overexpression of MDA. In certain embodiments, the tumor is selected from the group consisting of melanoma, glioblastoma multiforme, anaplastic astrocytoma, ependymoma, meningioma, oligodendroglioma, and combinations thereof. In certain embodiments, the tumor is melanoma.

Furthermore, the presently disclosed subject matter provides methods of using a CAR, an immunoresponsive cell or a pharmaceutical composition disclosed herein for increasing or lengthening survival of a subject having neoplasia. For example, and not by way of limitation, the presently disclosed subject matter provides methods of increasing or lengthening survival of a subject having neoplasia, wherein the method comprises administering an effective amount of a presently disclosed CAR, a presently disclosed immunoresponsive cell, or a presently disclosed pharmaceutical composition to the subject, thereby increasing or lengthening survival of the subject.

In certain embodiments, the neoplasia is associated with overexpression of MDA. In certain embodiments, the neoplasia is selected from the group consisting of melanoma, glioblastoma multiforme, anaplastic astrocytoma, ependymoma, meningioma, oligodendroglioma and combinations thereof. In certain embodiments, the neoplasia is melanoma. In certain embodiments, the method reduces or eradicates tumor burden in the subject.

In certain embodiments, the subject is a human. In certain embodiments, the immunoresponsive cell is a T cell.

The presently disclosed subject matter also provides methods for producing an immunoresponsive cell that binds to an MDA polypeptide. In certain embodiments, the method comprises introducing into the immunoresponsive cell a nucleic acid sequence that encodes the above-described CAR.

The presently disclosed subject matter further provides kits for treating a neoplasia, comprising a presently disclosed CAR, at least one presently disclosed immunoresponsive cell, or a presently disclosed pharmaceutical composition. In certain embodiments, the kit further includes written instructions for using the CAR, immunoresponsive cell or pharmaceutical composition for treating a neoplasia. In certain embodiments, the neoplasia is associated with overexpression of MDA. In certain embodiments, the neoplasia is selected from the group consisting of melanoma, glioblastoma multiforme, anaplastic astrocytoma, ependymoma, meningioma, oligodendroglioma and combinations thereof. In certain embodiments, the neoplasia is melanoma.

### BRIEF DESCRIPTION OF THE FIGURES

The following Detailed Description, given by way of example but not intended to limit the subject matter to specific embodiments described, may be understood in conjunction with the accompanying drawings.

FIG. 1 depicts a chimeric antigen receptor in accordance with one non-limiting embodiment of the presently disclosed subject matter.

FIG. 2 depicts Trp1 expression profile. B16, B78H1, and B78H1 engineered to express Trp1 on the surface were stained with TA99, TA99 single chain variable region fused to an Fc domain, or isotype control. The cell lines were washed and stained with anti-mouse IgG PE and further analyzed by flow cytometry. B16 was permeabilized before staining since the level of Trp1 expression is suboptimal in the surface in vitro.

FIG. 3 depicts transgene expression profile. Purified CD8⁺ T cells were activated with anti-CD3/CD28 for 2 days. Cells were transduced with supernatants from Plat-E cell lines on 2 consecutive days in the presence of protamine sulfate. On day 3, cells were analyzed for transgene expression by flow cytometry.

FIG. 4 depicts antitumor activity of T cells transduced with a CAR in accordance with the presently disclosed subject matter. B78H1 (CVT low) and B78H1-Trp1 (CVT high) were incubated with cells transduced with TCR or CAR as shown above at 1:10 ratio overnight. The target cells were washed extensively to remove T cells, trypsinized, and analyzed by flow cytometry. Dead cells were gated out using a viability dye. Measurement of killing of B16 relative to B78H1 was done in triplicate. Means and errors bars are shown. Numbers depict p values.

FIG. 5 depicts the imaging of immune tumor infiltration over time. (Right) C57BL/6 mice (5/group) were inoculated in the flank with B16 cells. After 3 week, mice were injected with cyclophosphamide (CTX). The next day, mice were injected with Trp1 TCR-luciferase CD4⁺ T cells. Imaging was performed 3-4 times weekly. Mean of photons/second/mm2 is plotted. Standard error of the means are represented (Left). Representative image at day 14 after treatment.

FIG. 6 depicts efficacy of Trp1-CAR T cells in vivo. Mice (10/group) bearing B16 melanoma tumors were pre-conditioned with cyclophosphamide (250 mg/Kg). The next day, mice were injected with either Mig (empty control vector) or Trp1-CAR transduced CD4⁺ and CD8⁺ T cells. Tumor size progression was measured over time and represented on the graph. Data points represent average size with standard error of the mean per group.

FIG. 7 depicts efficacy of Trp1-CAR T cells in vivo. Mice (10/group) bearing B16 melanoma were preconditioned with cyclophosphamide (250 mg/Kg). The next day, the mice were adoptively transferred with CD4⁺ and CD8⁺ T cells transduced with either Trp1-TCR or Trp1-CAR. Tumor size progression was measured over time and is represent on the graph.

FIGS. 8A and 8B depict persistence of CAR T cells in vivo. Mice bearing B16 melanoma (9-10/group) where preconditioned with 250 mg/Kg 21 days after tumor challenge. The next day, 100,000 CD4⁺ and CD8⁺ T cells transduced with Trp1-CAR or controls were administered through intravenous injection. After 7 days, the mice were bled retro-orbitally and lymphocytes in the blood were analyzed by flow cytometry before and after treatment. Events were gated on live CD4 and CD8. FIG. 8A depicts one representative plot. FIG. 8B depicts percent of GFP (transduced with CARs or empty vector) 7 days after treatment. Cells transduced with TCR-Trp1 serve as control. Bars represent means and standard error of the mean.

### DETAILED DESCRIPTION OF THE SUBJECT MATTER

The presently disclosed subject matter generally provides antigen-binding proteins chimeric antigen receptors (CARs) targeting MDA (e.g., Trp1). In certain embodiments, the CAR comprises an extracellular antigen-binding domain, a transmembrane domain and an intracellular domain, wherein the extracellular antigen-binding domain specifically binds to a an MDA polypeptide (e.g., a Trp1 polypeptide).

In certain embodiments, the CAR comprises an extracellular antigen-binding domain, a transmembrane domain and an intracellular domain, wherein the extracellular antigen-binding domain cross-competes for binding to an MDA polypeptide with a reference antibody or an antigen-binding portion thereof comprising a heavy chain variable region CDR1 comprising amino acids having the sequence set forth in SEQ ID NO: 1; a heavy chain variable region CDR2 comprising amino acids having the sequence set forth in SEQ ID NO: 2; a heavy chain variable region CDR3 comprising amino acids having the sequence set forth in SEQ ID NO: 3; a light chain variable region CDR1 comprising amino acids having the sequence set forth in SEQ ID NO: 4; a light chain variable region CDR2 comprising amino acids having the sequence set forth in SEQ ID NO: 5; and a light chain variable region CDR3 comprising amino acids having the sequence set forth in SEQ ID NO: 6. In certain embodiments, the CAR comprises an extracellular antigen-binding domain, a transmembrane domain and an intracellular domain, where the extracellular antigen-binding domain binds to the same epitope on an MDA polypeptide as a reference antibody or an antigen-binding portion thereof comprising a heavy chain variable region CDR1 comprising amino acids having the sequence set forth in SEQ ID NO: 1; a heavy chain variable region CDR2 comprising amino acids having the sequence set forth in SEQ ID NO: 2; a heavy chain variable region CDR3 comprising amino acids having the sequence set forth in SEQ ID NO: 3; a light chain variable region CDR1 comprising amino acids having the sequence set forth in SEQ ID NO: 4; a light chain variable region CDR2 comprising amino acids having the sequence set forth in SEQ ID NO: 5; and a light chain variable region CDR3 comprising amino acids having the sequence set forth in SEQ ID NO: 6.

The presently disclosed subject matter further provides immunoresponsive cells (e.g., a T cell (e.g., a cytotoxic T lymphocyte (CTL), a regulatory T cell, a central memory T cell, etc.), a Natural Killer (NK) cell, a human embryonic stem cell, a lymphoid progenitor cell, a T cell-precursor cell, and a pluripotent stem cell from which lymphoid cells may be differentiated) expressing the MDA-targeted CARs, and methods of using such immunoresponsive cells for treating a tumor, e.g., melanoma.

### I. Definitions

Unless defined otherwise, all technical and scientific terms used herein have the meaning commonly understood by a person skilled in the art to which this subject matter belongs. The following references provide one of skill with a general definition of many of the terms used in this subject matter: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, the following terms have the meanings ascribed to them below, unless specified otherwise.

As used herein, the term “about” or “approximately” means within an acceptable error range for the particular value as determined by one of ordinary skill in the art, which will depend in part on how the value is measured or determined, i.e., the limitations of the measurement system. For example, “about” can mean within 3 or more than 3 standard deviations, per the practice in the art. Alternatively, “about” can mean a range of up to 20%, preferably up to 10%, more preferably up to 5%, and more preferably still up to 1% of a given value. Alternatively, particularly with respect to biological systems or processes, the term can mean within an order of magnitude, preferably within 5-fold, and more preferably within 2-fold, of a value.

As used herein, the term “cell population” refers to a group of at least two cells expressing similar or different phenotypes. In non-limiting examples, a cell population can include at least about 10, at least about 100, at least about 200, at least about 300, at least about 400, at least about 500, at least about 600, at least about 700, at least about 800, at least about 900, at least about 1000 cells expressing similar or different phenotypes.

As used herein, the term “antibody” means not only intact antibody molecules, but also fragments of antibody molecules that retain immunogen-binding ability. Such fragments are also well known in the art and are regularly employed both in vitro and in vivo. Accordingly, as used herein, the term “antibody” means not only intact immunoglobulin molecules but also the well-known active fragments F(ab′)₂, and Fab. F(ab′)₂, and Fab fragments that lack the Fc fragment of intact antibody, clear more rapidly from the circulation, and may have less non-specific tissue binding of an intact antibody (Wahl et al., J. Nucl. Med. 24:316-325 (1983)). The antibodies of the presently disclosed subject matter comprise whole native antibodies, bispecific antibodies; chimeric antibodies; Fab, Fab′, single chain V region fragments (scFv), fusion polypeptides, and unconventional antibodies. In certain embodiments, an antibody is a glycoprotein comprising at least two heavy (H) chains and two light (L) chains inter-connected by disulfide bonds. Each heavy chain is comprised of a heavy chain variable region (abbreviated herein as V_{H}) and a heavy chain constant (C_{H}) region. The heavy chain constant region is comprised of three domains, CH1, CH2 and CH3. Each light chain is comprised of a light chain variable region (abbreviated herein as V_{L}) and a light chain constant C_{L} region. The light chain constant region is comprised of one domain, C_{L}. The V_{H} and V_{L} regions can be further sub-divided into regions of hypervariability, termed complementarity determining regions (CDR), interspersed with regions that are more conserved, termed framework regions (FR). Each V_{H} and V_{L} is composed of three CDRs and four FRs arranged from amino-terminus to carboxy-terminus in the following order: FR1, CDR1, FR2, CDR2, FR3, CDR3, FR4. The variable regions of the heavy and light chains contain a binding domain that interacts with an antigen. The constant regions of the antibodies may mediate the binding of the immunoglobulin to host tissues or factors, including various cells of the immune system (e.g., effector cells) and the first component (C1 q) of the classical complement system.

As used herein interchangeably, the terms “antigen-binding portion”, “antigen-binding fragment”, or “antigen-binding region” of an antibody, refer to the region or portion of an antibody that binds to the antigen and which confers antigen specificity to the antibody; fragments of antigen-binding proteins, for example, antibodies includes one or more fragments of an antibody that retain the ability to specifically bind to an antigen (e.g., an peptide/HLA complex). It has been shown that the antigen-binding function of an antibody can be performed by fragments of a full-length antibody. Examples of antigen-binding portions encompassed within the term “antibody fragments” of an antibody include a Fab fragment, a monovalent fragment consisting of the V_{L}, V_{H}, C_{L} and CH1 domains; a F(ab)₂ fragment, a bivalent fragment comprising two Fab fragments linked by a disulfide bridge at the hinge region; a Fd fragment consisting of the V_{H} and CH1 domains; a Fv fragment consisting of the V_{L} and V_{H} domains of a single arm of an antibody; a dAb fragment (Ward et al., 1989 Nature 341:544-546), which consists of a V_{H} domain; and an isolated complementarity determining region (CDR).

Furthermore, although the two domains of the Fv fragment, V_{L} and V_{H}, are coded for by separate genes, they can be joined, using recombinant methods, by a synthetic linker that enables them to be made as a single protein chain in which the V_{L} and V_{H} regions pair to form monovalent molecules. These are known as single chain Fv (scFv); see e.g., Bird et al., 1988 Science 242:423-426; and Huston et al., 1988 Proc. Natl. Acad. Sci. 85:5879-5883. These antibody fragments are obtained using conventional techniques known to those of ordinary skill in the art, and the fragments are screened for utility in the same manner as are intact antibodies.

An “isolated antibody” or “isolated antigen-binding protein” is one which has been identified and separated and/or recovered from a component of its natural environment. “Synthetic antibodies” or “recombinant antibodies” are generally generated using recombinant technology or using peptide synthetic techniques known to those of skill in the art.

As used herein, the term “single-chain variable fragment” or “scFv” is a fusion protein of the variable regions of the heavy (V_{H}) and light chains (V_{L}) of an immunoglobulin (e.g., mouse or human) covalently linked to form a V_{H}:V_{L} heterodimer. The heavy (V_{H}) and light chains (V_{L}) are either joined directly or joined by a peptide-encoding linker (e.g., about 10, 15, 20, 25 amino acids), which connects the N-terminus of the V_{H} with the C-terminus of the V_{L}, or the C-terminus of the V_{H} with the N-terminus of the V_{L}. The linker is usually rich in glycine for flexibility, as well as serine or threonine for solubility. The linker can link the heavy chain variable region and the light chain variable region of the extracellular antigen-binding domain. In certain embodiments, the linker comprises amino acids having the sequence set forth in SEQ ID NO: 11 as provided below.

[SEQ ID NO: 11]
GGGGSGGGGSGGGGS

In certain embodiments, the nucleic acid sequence encoding the amino acid sequence of SEQ ID NO: 11 is set forth in SEQ ID NO:26, which is provided below:

[SEQ ID NO: 26]
GGTGGAGGTGGATCAGGTGGAGGTGGATCTGGTGGAGGTGGATCT

Despite removal of the constant regions and the introduction of a linker, scFv proteins retain the specificity of the original immunoglobulin. Single chain Fv polypeptide antibodies can be expressed from a nucleic acid comprising V_{H}- and V_{L}-encoding sequences as described by Huston, et al. (Proc. Nat. Acad. Sci. USA, 85:5879-5883, 1988). See, also, U.S. Pat. Nos. 5,091,513, 5,132,405 and 4,956,778; and U.S. Patent Publication Nos. 2005/0196754 and 20050196754. Antagonistic scFvs having inhibitory activity have been described (see, e.g., Zhao et al., Hyrbidoma (Larchmt) 2008 27(6):455-51; Peter et al., J Cachexia Sarcopenia Muscle 2012 Aug. 12; Shieh et al., J Imunol 2009 183(4):2277-85; Giomarelli et al., Thromb Haemost 2007 97(6):955-63; Fife eta., J Clin Invst 2006 116(8):2252-61; Brocks et al., Immunotechnology 1997 3(3):173-84; Moosmayer et al., Ther Immunol 1995 2(10:31-40). Agonistic scFvs having stimulatory activity have been described (see, e.g., Peter et al., J Bioi Chern 2003 25278(38):36740-7; Xie et al., Nat Biotech 1997 15(8):768-71; Ledbetter et al., Crit Rev Immunol 1997 17(5-6):427-55; Ho et al., BioChim Biophys Acta 2003 1638(3):257-66).

As used herein, “F(ab)” refers to a fragment of an antibody structure that binds to an antigen but is monovalent and does not have a Fc portion, for example, an antibody digested by the enzyme papain yields two F(ab) fragments and an Fc fragment (e.g., a heavy (H) chain constant region; Fc region that does not bind to an antigen).

As used herein, “F(ab′)₂” refers to an antibody fragment generated by pepsin digestion of whole IgG antibodies, wherein this fragment has two antigen binding (ab′) (bivalent) regions, wherein each (ab′) region comprises two separate amino acid chains, a part of a H chain and a light (L) chain linked by an S—S bond for binding an antigen and where the remaining H chain portions are linked together. A “F(ab′)₂” fragment can be split into two individual Fab′ fragments.

As used herein, the term “vector” refers to any genetic element, such as a plasmid, phage, transposon, cosmid, chromosome, virus, virion, etc., which is capable of replication when associated with the proper control elements and which can transfer gene sequences into cells. Thus, the term includes cloning and expression vehicles, as well as viral vectors and plasmid vectors.

As used herein, the term “expression vector” refers to a recombinant nucleic acid sequence, e.g., a recombinant DNA molecule, containing a desired coding sequence and appropriate nucleic acid sequences necessary for the expression of the operably linked coding sequence in a particular host organism. Nucleic acid sequences necessary for expression in prokaryotes usually include a promoter, an operator (optional), and a ribosome binding site, often along with other sequences. Eukaryotic cells are known to utilize promoters, enhancers, and termination and polyadenylation signals.

As used herein, “CDRs” are defined as the complementarity determining region amino acid sequences of an antibody which are the hypervariable regions of immunoglobulin heavy and light chains. See, e.g., Kabat et al., Sequences of Proteins of Immunological Interest, 4th U.S. Department of Health and Human Services, National Institutes of Health (1987). Generally, antibodies comprise three heavy chain and three light chain CDRs or CDR regions in the variable region. CDRs provide the majority of contact residues for the binding of the antibody to the antigen or epitope. In certain embodiments, the CDRs regions are delineated using the Kabat system (Kabat, E. A., et al. (1991) Sequences of Proteins of Immunological Interest, Fifth Edition, U.S. Department of Health and Human Services, NIH Publication No. 91-3242).

As used herein, the term “affinity” is meant a measure of binding strength. Affinity may depend on the closeness of stereochemical fit between antibody combining sites and antigen determinants, on the size of the area of contact between them, and on the distribution of charged and hydrophobic groups. Affinity also includes the term “avidity,” which refers to the strength of the antigen-antibody bond after formation of reversible complexes. Methods for calculating the affinity of an antibody for an antigen are known in the art, comprising use of binding experiments to calculate affinity. Antibody activity in functional assays (e.g., flow cytometry assay) is also reflective of antibody affinity. Antibodies and affinities can be phenotypically characterized and compared using functional assays (e.g., flow cytometry assay).

Nucleic acid molecules useful in the presently disclosed subject matter include any nucleic acid molecule that encodes a polypeptide or a fragment thereof. In certain embodiments, nucleic acid molecules useful in the presently disclosed subject matter include nucleic acid molecules that encode an antibody or an antigen-binding portion thereof. Such nucleic acid molecules need not be 100% identical with an endogenous nucleic acid sequence, but will typically exhibit substantial identity. Polynucleotides having “substantial homology” or “substantial identity” to an endogenous sequence are typically capable of hybridizing with at least one strand of a double-stranded nucleic acid molecule. By “hybridize” is meant pair to form a double-stranded molecule between complementary polynucleotide sequences (e.g., a gene described herein), or portions thereof, under various conditions of stringency. (See, e.g., Wahl, G. M. and S. L. Berger (1987) Methods Enzymol. 152:399; Kimmel, A. R. (1987) Methods Enzymol. 152:507).

For example, stringent salt concentration will ordinarily be less than about 750 mM NaCl and 75 mM trisodium citrate, preferably less than about 500 mM NaCl and 50 mM trisodium citrate, and more preferably less than about 250 mM NaCl and 25 mM trisodium citrate. Low stringency hybridization can be obtained in the absence of organic solvent, e.g., formamide, while high stringency hybridization can be obtained in the presence of at least about 35% formamide, and more preferably at least about 50% formamide. Stringent temperature conditions will ordinarily include temperatures of at least about 30° C., more preferably of at least about 37° C., and most preferably of at least about 42° C. Varying additional parameters, such as hybridization time, the concentration of detergent, e.g., sodium dodecyl sulfate (SDS), and the inclusion or exclusion of carrier DNA, are well known to those skilled in the art. Various levels of stringency are accomplished by combining these various conditions as needed. In a preferred: embodiment, hybridization will occur at 30° C. in 750 mM NaCl, 75 mM trisodium citrate, and 1% SDS. In a more preferred embodiment, hybridization will occur at 37° C. in 500 mM NaCl, 50 mM trisodium citrate, 1% SDS, 35% formamide, and 100 μg/ml denatured salmon sperm DNA (ssDNA). In a most preferred embodiment, hybridization will occur at 42° C. in 250 mM NaCl, 25 mM trisodium citrate, 1% SDS, 50% formamide, and 200 μg/ml ssDNA. Useful variations on these conditions will be readily apparent to those skilled in the art.

For most applications, washing steps that follow hybridization will also vary in stringency. Wash stringency conditions can be defined by salt concentration and by temperature. As above, wash stringency can be increased by decreasing salt concentration or by increasing temperature. For example, stringent salt concentration for the wash steps will preferably be less than about 30 mM NaCl and 3 mM trisodium citrate, and most preferably less than about 15 mM NaCl and 1.5 mM trisodium citrate. Stringent temperature conditions for the wash steps will ordinarily include a temperature of at least about 25° C., more preferably of at least about 42° C., and even more preferably of at least about 68° C. In a preferred embodiment, wash steps will occur at 25° C. in 30 mM NaCl, 3 mM trisodium citrate, and 0.1% SDS. In a more preferred embodiment, wash steps will occur at 42° C. in 15 mM NaCl, 1.5 mM trisodium citrate, and 0.1% SDS. In a more preferred embodiment, wash steps will occur at 68° C. in 15 mM NaCl, 1.5 mM trisodium citrate, and 0.1% SDS. Additional variations on these conditions will be readily apparent to those skilled in the art. Hybridization techniques are well known to those skilled in the art and are described, for example, in Benton and Davis (Science 196:180, 1977); Grunstein and Rogness (Proc. Natl. Acad. Sci., USA 72:3961, 1975); Ausubel et al. (Current Protocols in Molecular Biology, Wiley Interscience, New York, 2001); Berger and Kimmel (Guide to Molecular Cloning Techniques, 1987, Academic Press, New York); and Sambrook et al., Molecular Cloning: A Laboratory Manual, Cold Spring Harbor Laboratory Press, New York.

The terms “substantially homologous” or “substantially identical” mean a polypeptide or nucleic acid molecule that exhibits at least 50% homology or identity to a reference amino acid sequence (for example, any one of the amino acid sequences described herein) or nucleic acid sequence (for example, any one of the nucleic acid sequences described herein). For example, such a sequence is at least 60%, 65%, 70%, 75%, 80%, 85%, 90%, 95% or even 99% homologous or identical at the amino acid level or nucleic acid to the sequence used for comparison.

Sequence homology or sequence identity is typically measured using sequence analysis software (for example, Sequence Analysis Software Package of the Genetics Computer Group, University of Wisconsin Biotechnology Center, 1710 University Avenue, Madison, Wis. 53705, BLAST, BESTFIT, GAP, or PILEUP/PRETTYBOX programs). Such software matches identical or similar sequences by assigning degrees of homology to various substitutions, deletions, and/or other modifications. In an exemplary approach to determining the degree of identity, a BLAST program may be used, with a probability score between e⁻³ and e⁻¹⁰⁰ indicating a closely related sequence.

In certain embodiments, the term “cross-compete” or “compete” refers to the situation where binding of an extracellular antigen-binding domain of a presently disclosed CAR to a given antigen or a given polypeptide, i.e., an MDA (e.g., Trp1), decreases or reduces binding of a reference antibody or an antigen-binding portion thereof, e.g., that comprises the V_{H} and V_{L} CDR1, CDR2, and CDR3 sequences or V_{H} and V_{L} sequences disclosed in Table 1, to the same antigen, i.e., an MDA (e.g., Trp1). The term “cross-compete” or “compete” also refers to the situation where binding of a reference antibody or an antigen-binding portion thereof to a given antigen or a given polypeptide, i.e., an MDA (e.g., Trp1), decreases or reduces binding of an extracellular antigen-binding domain of a presently disclosed CAR to the same antigen. In certain embodiments, the “cross-competing” or “competing” extracellular antigen-binding domain binds to the same or substantially the same epitope, an overlapping epitope, or an adjacent epitope on an MDA (e.g., Trp1) as the reference antibody or antigen-binding portion thereof.

As used herein, the term “analog” refers to a structurally related polypeptide or nucleic acid molecule having the function of a reference polypeptide or nucleic acid molecule.

As used herein, the term “ligand” refers to a molecule that binds to a receptor. In particular, the ligand binds a receptor on another cell, allowing for cell-to-cell recognition and/or interaction.

As used herein, the term “disease” refers to any condition or disorder that damages or interferes with the normal function of a cell, tissue, or organ. Examples of diseases include neoplasia or pathogen infection of cell.

An “effective amount” (or “therapeutically effective amount”) is an amount sufficient to affect a beneficial or desired clinical result upon treatment. An effective amount can be administered to a subject in one or more doses. In terms of treatment, an effective amount is an amount that is sufficient to palliate, ameliorate, stabilize, reverse or slow the progression of the disease (e.g., a neoplasia), or otherwise reduce the pathological consequences of the disease (e.g., a neoplasia). The effective amount is generally determined by the physician on a case-by-case basis and is within the skill of one in the art. Several factors are typically taken into account when determining an appropriate dosage to achieve an effective amount. These factors include age, sex and weight of the subject, the condition being treated, the severity of the condition and the form and effective concentration of the immunoresponsive cells administered.

As used herein, the term “neoplasia” refers to a disease characterized by the pathological proliferation of a cell or tissue and its subsequent migration to or invasion of other tissues or organs. Neoplasia growth is typically uncontrolled and progressive, and occurs under conditions that would not elicit, or would cause cessation of, multiplication of normal cells. Neoplasias can affect a variety of cell types, tissues, or organs, including but not limited to an organ selected from the group consisting of skin, bladder, colon, bone, brain, breast, cartilage, glia, esophagus, fallopian tube, gallbladder, heart, intestines, kidney, liver, lung, lymph node, nervous tissue, ovaries, pleura, pancreas, prostate, skeletal muscle, spinal cord, spleen, stomach, testes, thymus, thyroid, trachea, urogenital tract, ureter, urethra, uterus, and vagina, or a tissue or cell type thereof. Neoplasias include cancers, such as melanoma, sarcomas, carcinomas, or plasmacytomas (malignant tumor of the plasma cells).

As used herein, the term “heterologous nucleic acid molecule or polypeptide” refers to a nucleic acid molecule (e.g., a cDNA, DNA or RNA molecule) or polypeptide that is not normally present in a cell or sample obtained from a cell. This nucleic acid may be from another organism, or it may be, for example, an mRNA molecule that is not normally expressed in a cell or sample.

As used herein, the term “immunoresponsive cell” refers to a cell that functions in an immune response or a progenitor, or progeny thereof.

As used herein, the term “modulate” refers positively or negatively alter. Exemplary modulations include an about 1%, about 2%, about 5%, about 10%, about 25%, about 50%, about 75%, or about 100% change.

As used herein, the term “increase” refers to alter positively by at least about 5%, including, but not limited to, alter positively by about 5%, by about 10%, by about 25%, by about 30%, by about 50%, by about 75%, or by about 100%.

As used herein, the term “reduce” refers to alter negatively by at least about 5% including, but not limited to, alter negatively by about 5%, by about 10%, by about 25%, by about 30%, by about 50%, by about 75%, or by about 100%.

As used herein, the term “isolated cell” refers to a cell that is separated from the molecular and/or cellular components that naturally accompany the cell.

As used herein, the term “isolated,” “purified,” or “biologically pure” refers to material that is free to varying degrees from components which normally accompany it as found in its native state. “Isolate” denotes a degree of separation from original source or surroundings. “Purify” denotes a degree of separation that is higher than isolation. A “purified” or “biologically pure” protein is sufficiently free of other materials such that any impurities do not materially affect the biological properties of the protein or cause other adverse consequences. That is, a nucleic acid or polypeptide of the presently disclosed subject matter is purified if it is substantially free of cellular material, viral material, or culture medium when produced by recombinant DNA techniques, or chemical precursors or other chemicals when chemically synthesized. Purity and homogeneity are typically determined using analytical chemistry techniques, for example, polyacrylamide gel electrophoresis or high performance liquid chromatography. The term “purified” can denote that a nucleic acid or protein gives rise to essentially one band in an electrophoretic gel. For a protein that can be subjected to modifications, for example, phosphorylation or glycosylation, different modifications may give rise to different isolated proteins, which can be separately purified.

As used herein, the term “secreted” refers to a polypeptide that is released from a cell via the secretory pathway through the endoplasmic reticulum, Golgi apparatus, and as a vesicle that transiently fuses at the cell plasma membrane, releasing the proteins outside of the cell.

As used herein, the term “specifically binds” or “specifically binds to” or “specifically target” refers to a polypeptide or fragment thereof (e.g., the extracellular antigen-binding domain of the CAR) that recognizes and binds to a biological molecule of interest (e.g., a polypeptide), but which does not substantially recognize and bind other molecules in a sample, for example, a biological sample, which includes or expresses an MDA (e.g., human MDA or mouse MDA), e.g., Trp1 (e.g., human Trp1 or mouse Trp1).

As used herein, the term “treating” or “treatment” refers to clinical intervention in an attempt to alter the disease course of the individual or cell being treated, and can be performed either for prophylaxis or during the course of clinical pathology. Therapeutic effects of treatment include, without limitation, preventing occurrence or recurrence of disease, alleviation of symptoms, diminishment of any direct or indirect pathological consequences of the disease, preventing metastases, decreasing the rate of disease progression, amelioration or palliation of the disease state, and remission or improved prognosis. By preventing progression of a disease or disorder, a treatment can prevent deterioration due to a disorder in an affected or diagnosed subject or a subject suspected of having the disorder, but also a treatment may prevent the onset of the disorder or a symptom of the disorder in a subject at risk for the disorder or suspected of having the disorder.

As used herein, the term “subject” refers to any animal (e.g., a mammal), including, but not limited to, humans, non-human primates, rodents, and the like (e.g., which is to be the recipient of a particular treatment, or from whom cells are harvested).

### II. Melanoma Differentiation Antigens

Genes that encode melanoma antigens recognized by tumor-infiltrating lymphocytes (TIL) have been identified (Rosenberg. Immunol. Today (1997); 18: 175). With the exception of melanocytes and retina, normal tissues do not express these antigens, and no expression of these genes has been observed in cancers other than melanoma. Hence, these antigens represent molecules associated with the melanocyte lineage and are called melanoma differentiation antigens (MDA). MDA are reckoned to be tumor rejection antigens as TIL targeting MDA were associated with in vivo tumor regression when adoptively transferred to patients with metastatic melanoma (Rosenberg, et al. N. Engl. J. Med. (1988); 319: 1676).

In certain embodiments, the MDA is selected from the group consisting of Tyrosinase related protein 1 (“TRP1”), tyrosinase, Melan-A, gp100, and TRP2. Tyrosinase is also known as OCA1 or SKC35. Melan-A is also known as MART-1. gp100 is also known as D10H12S53E, D12S53Eh, gp87, Pmel17, or Si. TRP2 is also known as TRP-2, tyrosinase-related protein-2, Tyrp2, Tyrp-2, or DCT.

In certain embodiments, the MDA is Trp1. TRP1 (also known as TRP, TRP-1, CAS2, CATB, GP75, OCA3, TRP1, TYRP, TYRP1, b-PROTEIN) encodes a melanosomal enzyme of the tyrosinase family, and is involved in melanin synthesis. Additionally, Trp1 is involved in stabilizing and modulating tyrosinase protein, and affects melanosome structure and melanocyte proliferation. Defects in this gene can cause rufous oculocutaneous albinism and oculocutaneous albinism type III (OCA3).

A well defined TCR recognizing Trp1 is available. This Trp1-recognizing TCR can be used for comparison with a Trp1-expressing CAR side by side.

### III. Chimeric Antigen Receptor (CAR)

Chimeric antigen receptors (CARs) are engineered receptors, which graft or confer a specificity of interest onto an immune effector cell. CARs can be used to graft the specificity of a monoclonal antibody onto a T cell; with transfer of their coding sequence facilitated by retroviral vectors.

There are at least three generations of CARS. “First generation” CARs are typically composed of an extracellular antigen binding domain (e.g., a single-chain variable fragments (scFv)) fused to a transmembrane domain, fused to cytoplasmic/intracellular domain of the T cell receptor chain. “First generation” CARs typically have the intracellular domain from the CD3zeta-chain, which is the primary transmitter of signals from endogenous TCRs. “First generation” CARs can provide de novo antigen recognition and cause activation of both CD4⁺ and CD8⁺ T cells through their CD3zeta chain signaling domain in a single fusion molecule, independent of HLA-mediated antigen presentation. “Second generation” CARs add intracellular domains from various co-stimulatory molecules (e.g., CD28, 4-1BB, ICOS, OX40) to the cytoplasmic tail of the CAR to provide additional signals to the T cell. “Second generation” CARs comprise those that provide both co-stimulation (e.g., CD28 or 4-1BB) and activation (CD3zeta). Preclinical studies have indicated that “Second Generation” CARs can improve the anti-tumor activity of T cells. For example, robust efficacy of “Second Generation” CAR modified T cells was demonstrated in clinical trials targeting the CD19 molecule in patients with chronic lymphoblastic leukemia (CLL) and acute lymphoblastic leukemia (ALL). “Third generation” CARs comprise those that provide multiple co-stimulation (e.g., CD28 and 4-1BB) and activation (CD3zeta).

In certain non-limiting embodiments, the extracellular antigen-binding domain of a presently disclosed CAR cross-reacts with both human and mouse MDA (e.g., both human and mouse Trp1). In certain non-limiting embodiments, the extracellular antigen-binding domain of a presently disclosed CAR has a high binding specificity as well as high binding affinity to both mouse and human MDA (e.g., mouse and human Trp1). For example, in such embodiments, the extracellular antigen-binding domain of the CAR (embodied, for example, an scFv or an analog thereof) binds to an MDA polypeptide (e.g., a Trp1 polypeptide) with a dissociation constant (K_{d}) of about 2×10⁻⁷ M or less. In certain embodiments, the K_{d} is about 2×10⁻⁷ M or less, about 1×10⁻⁷ M or less, about 9×10⁻⁸ M or less, about 1×10⁻⁸ M or less, about 9×10⁻⁹ M or less, about 5×10⁻⁹ M or less, about 4×10⁻⁹ M or less, about 3×10⁻⁹ M or less, about 2×10⁻⁹ M or less, or about 1×10⁻⁹ M or less. In certain non-limiting embodiments, the K_{d} is from about 3×10⁻⁹ M or less. In certain non-limiting embodiments, the K_{d} is from about 1×10⁻⁹ M to about 3×10⁻⁷ M. In certain non-limiting embodiments, the K_{d} is from about 1.5×10⁻⁹ M to about 3×10⁻⁷ M. In certain non-limiting embodiments, the K_{d} is from about 1.5×10⁻⁹ M to about 2.7×10⁻⁷ M.

Binding of the extracellular antigen-binding domain (for example, an scFv or an analog thereof) of a presently disclosed MDA-targeted CAR can be confirmed by, for example, enzyme-linked immunosorbent assay (ELISA), radioimmunoassay (RIA), FACS analysis, bioassay (e.g., growth inhibition), or Western Blot assay. Each of these assays generally detect the presence of protein-antibody complexes of particular interest by employing a labeled reagent (e.g., an antibody, or a scFv) specific for the complex of interest. For example, the scFv can be radioactively labeled and used in a radioimmunoassay (RIA) (see, for example, Weintraub, B., Principles of Radioimmunoassays, Seventh Training Course on Radioligand Assay Techniques, The Endocrine Society, March 1986, which is incorporated by reference herein). The radioactive isotope can be detected by such means as the use of a y counter or a scintillation counter or by autoradiography. In certain embodiments, the extracellular antigen-binding domain of the MDA-targeted CAR is labeled with a fluorescent marker. Non-limiting examples of fluorescent markers include green fluorescent protein (GFP), blue fluorescent protein (e.g., EBFP, EBFP2, Azurite, and mKalama1), cyan fluorescent protein (e.g., ECFP, Cerulean, and CyPet), and yellow fluorescent protein (e.g., YFP, Citrine, Venus, and YPet). In one embodiment, the scFv of a presently disclosed MDA-targeted CAR is labeled with GFP.

In accordance with the presently disclosed subject matter, the CARs comprise an extracellular antigen-binding domain, a transmembrane domain and an intracellular domain, wherein the extracellular antigen-binding domain specifically binds to an MDA (e.g., Trp1). In certain embodiments, the extracellular antigen-binding domain is an scFv. In certain embodiments, the extracellular antigen-binding domain is a Fab, which is optionally crosslinked. In a certain embodiments, the extracellular antigen-binding domain is a F(ab)₂. In certain embodiments, any of the foregoing molecules may be comprised in a fusion protein with a heterologous sequence to form the extracellular antigen-binding domain. In certain embodiments, the extracellular antigen-binding domain comprises a murine scFv that binds specifically to an MDA (e.g., Trp1). In certain embodiments, the extracellular antigen-binding domain comprises a human scFv that binds specifically to an MDA (e.g., Trp1). In certain embodiments, the scFv is identified by screening scFv phage library with MDA-Fc fusion proteins.

### Extracellular Antigen-Binding Domain of CAR

In certain embodiments, the extracellular antigen-binding domain specifically binds to an MDA polypeptide (e.g., a Trp1 polypeptide). In certain embodiments, the Trp1 polypeptide is a human Trp1 polypeptide. The human Trp1 polypeptide can have an amino acid sequence that is at least about 85%, about 90%, about 95%, about 96%, about 97%, about 98%, about 99% or 100% homologous to the sequence having a NCBI Reference No: NP_000541.1 (SEQ ID NO: 10, provided below), or fragments thereof, and/or may optionally comprise up to one or up to two or up to three conservative amino acid substitutions. In certain embodiments, the human Trp1 polypeptide can have an amino acid sequence that is a consecutive portion of SEQ ID NO:10 which is at least 20, or at least 30, or at least 40, or at least 50, or at least 100, or at least 200, or at least 300, or at least 400, or at least 500, and up to 537 amino acids in length. Alternatively or additionally, in non-limiting various embodiments, the human Trp1 polypeptide has an amino acid sequence of amino acids 1 to 537, 1 to 50, 50 to 100, 100 to 200, 200 to 300, 300 to 400, or 400 to 537 of SEQ ID NO: 10.

```
[SEQ ID NO: 10]
    1 MSAPKLLSLG CIFFPLLLFQ QARAQFPRQC ATVEALRSGM CCPDLSPVSG PGTDRCGSSS
  61 GRGRCEAVTA DSRPHSPQYP HDGRDDREVW PLRFFNRTCH CNGNFSGHNC GTCRPGWRGA
121 ACDQRVLIVR RNLLDLSKEE KNHFVRALDM AKRTTHPLFV IATRRSEEIL GPDGNTPQFE
181 NISIYNYFVW THYYSVKKTF LGVGQESFGE VDFSHEGPAF LTWHRYHLLR LEKDMQEMLQ
241 EPSFSLPYWN FATGKNVCDI CTDDLMGSRS NFDSTLISPN SVFSQWRVVC DSLEDYDTLG
301 TLCNSTEDGP IRRNPAGNVA RPMVQRLPEP QDVAQCLEVG LFDTPPFYSN STNSFRNTVE
361 GYSDPTGKYD PAVRSLHNLA HLFLNGTGGQ THLSPNDPIF VLLHTFTDAV FDEWLRRYNA
421 DISTFPLENA PIGHNRQYNM VPFWPPVTNT EMFVTAPDNL GYTYEIQWPS REFSVPEIIA
481 IAVVGALLLV ALIFGTASYL IRARRSMDEA NQPLLTDQYQ CYAEEYEKLQ NPNQSVV
```

In certain embodiments, the Trp1 polypeptide is a mouse/murine Trp1 polypeptide. The mouse Trp1 polypeptide can have an amino acid sequence that is at least about 85%, about 90%, about 95%, about 96%, about 97%, about 98%, about 99% or 100% homologous to the sequence having a NCBI Reference No: NP_001268944.1 (SEQ ID No: 37, provided below), or fragments thereof, and/or may optionally comprise up to one or up to two or up to three conservative amino acid substitutions. In certain embodiments, the mouse Trp1 polypeptide can have an amino acid sequence that is a consecutive portion of SEQ ID NO: 37 which is at least 20, or at least 30, or at least 40, or at least 50, or at least 100, or at least 200, or at least 300, or at least 400, or at least 500, and up to 537 amino acids in length. Alternatively or additionally, in non-limiting various embodiments, the mouse Trp1 polypeptide has an amino acid sequence of amino acids 1 to 537, 1 to 50, 50 to 100, 100 to 200, 200 to 300, 300 to 400, or 400 to 537 of SEQ ID NO: 37.

```
[SEQ ID NO: 37]
    1 MKSYNVLPLA YISLFLMLFY QVWAQFPREC ANIEALRRGV CCPDLLPSSG PGTDPCGSSS
  61 GRGRCVAVIA DSRPHSRHYP HDGKDDREAW PLRFFNRTCQ CNDNFSGHNC GTCRPGWRGA
121 ACNQKILTVR RNLLDLSPEE KSHFVRALDM AKRTTHPQFV IATRRLEDIL GPDGNTPQFE
181 NISVYNYFVW THYYSVKKTF LGTGQESFGD VDFSHEGPAF LTWHRYHLLQ LERDMQEMLQ
241 EPSFSLPYWN FATGKNVCDV CTDDLMGSRS NFDSTLISPN SVFSQWRVVC ESLEEYDTLG
301 TLCNSTEGGP IRRNPAGNVG RPAVQRLPEP QDVTQCLEVR VFDTPPFYSN STDSFRNTVE
361 GYSAPTGKYD PAVRSLHNLA HLFLNGTGGQ THLSPNDPIF VLLHTFTDAV FDEWLRRYNA
421 DISTFPLENA PIGHNRQYNM VPFWPPVTNT EMFVTAPDNL GYAYEVQWPG QEFTVSEIIT
481 IAVVAALLLV AAIFGVASCL IRSRSTKNEA NQPLLTDHYQ RYAEDYEELP NPNHSMV
```

In certain embodiments, the extracellular antigen-binding domain specifically binds to a human Trp1 polypeptide as well as a mouse Trp1 polypeptide.

In certain embodiments, the extracellular antigen-binding domain is an scFv. In certain embodiments, the scFv is a murine scFv. In certain embodiments, the scFv is a human scFv. In certain embodiments, the scFv is a humanized scFv. In certain embodiments, the scFv comprises amino acids having the sequence set forth in SEQ ID NO: 9, which is described in the following Table 1. In certain embodiments, the scFv is derived from the TA99 antibody disclosed in International Patent Publication No. WO96/40249, the content of which is herein incorporated by reference in its entirety.

In certain embodiments, the extracellular antigen-binding domain is an scFv, which comprises a heavy chain variable region (V_{H}) comprising amino acids having the sequence set forth in SEQ ID NO:7 and a light chain variable region (V_{L}) comprising amino acids having the sequence set forth in SEQ ID NO:8, optionally with (iii) a linker sequence, for example a linker peptide, between the heavy chain variable region and the light chain variable region. In one non-limiting embodiment, the linker comprises amino acids having the sequence set forth in SEQ ID NO:11. In certain embodiments, the extracellular antigen-binding domain is an scFv-Fc fusion protein or full length human IgG with V_{H} and V_{L} regions or CDRs selected from Table 1. In certain embodiments, the extracellular antigen-binding domain comprises a V_{H} comprising an amino acid sequence that is at least about 80% (e.g., at least about 85%, at least about 90%, or at least about 95%) homologous to the sequence set forth in SEQ ID NO: 7, as shown in Table 1. For example, the extracellular antigen-binding domain comprises a V_{H} comprising an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% homologous to the sequence set forth in SEQ ID NO: 7. In one non-limiting embodiment, the extracellular antigen-binding domain comprises a V_{H} comprising amino acids having the sequence set forth in SEQ ID NO:7. In certain embodiments, the extracellular antigen-binding domain comprises a V_{L} comprising an amino acid sequence that is at least about 80% (e.g., at least about 85%, at least about 90%, or at least about 95%) homologous to the sequence set forth in SEQ ID NO: 8, as shown in Table 1. For example, the extracellular antigen-binding domain comprises a V_{L} comprising an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% homologous to the sequence set forth in SEQ ID NO: 8. In one non-limiting embodiment, the extracellular antigen-binding domain comprises a V_{L} comprising amino acids having the sequence set forth in SEQ ID NO:8. In certain embodiments, the extracellular antigen-binding domain comprises a V_{H} comprising an amino acid sequence that is at least about 80% (e.g., at least about 85%, at least about 90%, or at least about 95%) homologous to the sequence set forth in SEQ ID NO: 7, and a V_{L} comprising an amino acid sequence that is at least about 80% (e.g., at least about 85%, at least about 90%, or at least about 95%) homologous to the sequence set forth in SEQ ID NO: 8. In certain embodiments, the extracellular antigen-binding domain comprises a V_{H} comprising amino acids having the sequence set forth in SEQ ID NO:7 and a V_{L} comprising amino acids having the sequence set forth in SEQ ID NO:8.

In certain embodiments, the extracellular antigen-binding domain comprises a V_{H} CDR1 comprising amino acids having the sequence set forth in SEQ ID NO:1 or a conservative modification thereof, a V_{H} CDR2 comprising amino acids having the sequence set forth in SEQ ID NO:2 or a conservative modification thereof, and a V_{H} CDR3 comprising amino acids having the sequence set forth in SEQ ID NO:3 or conservative modifications thereof, as shown in Table 1. In certain embodiments, the extracellular antigen-binding domain comprises a V_{H} CDR1 comprising amino acids having the sequence set forth in SEQ ID NO:1, a V_{H} CDR2 comprising amino acids having the sequence set forth in SEQ ID NO:2, and a V_{H} CDR3 comprising amino acids having the sequence set forth in SEQ ID NO:3.

In certain embodiments, the extracellular antigen-binding domain comprises a V_{L} CDR1 comprising amino acids having the sequence set forth in SEQ ID NO:4 or a conservative modification thereof, a V_{L} CDR2 comprising amino acids having the sequence set forth in SEQ ID NO: 5 or a conservative modification thereof, and a V_{L} CDR3 comprising amino acids having the sequence set forth in SEQ ID NO: 6 or a conservative modification thereof, as shown in Table 1. In certain embodiments, the extracellular antigen-binding domain comprises a V_{L} CDR1 comprising amino acids having the sequence set forth in SEQ ID NO:4, a V_{L} CDR2 comprising amino acids having the sequence set forth in SEQ ID NO: 5, and a V_{L} CDR3 comprising amino acids having the sequence set forth in SEQ ID NO: 6.

In certain embodiments, the extracellular antigen-binding domain comprises a V_{H} CDR1 comprising amino acids having the sequence set forth in SEQ ID NO: 1 or a conservative modification thereof, a V_{H} CDR2 comprising amino acids having the sequence set forth in SEQ ID NO: 2 or a conservative modification thereof, a V_{H} CDR3 comprising amino acids having the sequence set forth in SEQ ID NO: 3 or conservative modifications thereof, a V_{L} CDR1 comprising amino acids having the sequence set forth in SEQ ID NO: 4 or a conservative modification thereof, a V_{L} CDR2 comprising amino acids having the sequence set forth in SEQ ID NO: 5 or a conservative modification thereof, and a V_{L} CDR3 comprising amino acids having the sequence set forth in SEQ ID NO: 6 or a conservative modification thereof. In certain embodiments, the extracellular antigen-binding domain comprises a V_{H} CDR1 comprising amino acids having the sequence set forth in SEQ ID NO: 1, a V_{H} CDR2 comprising amino acids having the sequence set forth in SEQ ID NO: 2, a V_{H} CDR3 comprising amino acids having the sequence set forth in SEQ ID NO: 3, a V_{L} CDR1 comprising amino acids having the sequence set forth in SEQ ID NO: 4, a V_{L} CDR2 comprising amino acids having the sequence set forth in SEQ ID NO: 5, and a V_{L} CDR3 comprising amino acids having the sequence set forth in SEQ ID NO: 6.

TABLE 1

| Antigen | | A Trp1 polypeptide | | |
|---|---|---|---|---|
| CDRs | | 1 | 2 | 3 |
| V_{H} | a.a. | GFNIKDYFLH [SEQ ID | WINPDNGNTVYDPKFQG | DYTYEKAALDY [SEQ |
| | | NO: 1] | [SEQ ID NO: 2] | ID NO: 3] |
| | DNA | GGCTTCAACATTAAAGAC | TGGATTAATCCTGATAAT | GACTATACTTATGAAA |
| | | TACTTTTTACAC [SEQ | GGTAATACTGTTTATGAC | AGGCTGCTCTGGACTA |
| | | ID NO: 38] | CCGAAGTTTCAGGGC | C [SEQ ID NO: |
| | | | [SEQ ID NO: 39] | 40] |
| V_{L} | a.a. | RASGNIYNYLA [SEQ | DAKTLAD [SEQ ID | QHFWSLPFT [SEQ |
| | | ID NO: 4] | NO: 5] | ID NO: 6] |
| | DNA | CGAGCAAGTGGAAATATT | GATGCAAAAACCTTAGCA | CAACATTTTTGGAGTC |
| | | TACAATTATTTAGCA | GAT [SEQ ID NO: | TTCCATTCACG [SEQ |
| | | [SEQ ID NO: 41] | 42] | ID NO: 43] |

| | |
|---|---|
| Full V_{H} | MALPVTALLLPLALLLHAEVQLQQSGAELVRPGALVKLSCKTSGFNIKDYFLHWVRQ |
| | RPDQGLEWIGWINPDNGNTVYDPKFQGTASLTADTSSNTVYLQLSGLTSEDTAVYFC |
| | TRRDYTYEKAALDYWGQGASVIVSS [SEQ ID NO: 7] |
| DNA | ATGGCCTTACCAGTGACCGCCTTGCTCCTGCCGCTGGCCTTGCTGCTCCACGCCGAG |
| | GTTCAGCTGCAGCAGTCTGGGGCTGAGCTTGTGAGGCCAGGGGCCTTGGTCAAGTTG |
| | TCCTGCAAAACTTCTGGCTTCAACATTAAAGACTACTTTTTACACTGGGTGAGACAG |
| | AGGCCTGACCAGGGCCTGGAGTGGATTGGATGGATTAATCCTGATAATGGTAATACT |
| | GTTTATGACCCGAAGTTTCAGGGCACGGCCAGTTTAACAGCAGACACATCCTCCAAC |
| | ACAGTCTACTTGCAGCTCAGCGGCCTGACATCTGAGGACACTGCCGTCTATTTCTGT |
| | ACTCGGAGGGACTATACTTATGAAAAGGCTGCTCTGGACTACTGGGGTCAGGGAGCC |
| | TCAGTCATCGTCTCCTCA [SEQ ID NO: 27] |
| Full V_{L} | AFQMSQSPASLSASVGETVTITCRASGNIYNYLAWYQQKQGKSPHLLVYDAKTLADG |
| | VPSRFSGSGSGTQYSLKISSLQTEDSGNYYCQHFWSLPFTFGSGTKLEIKAAA |
| | [SEQ ID NO: 8] |
| DNA | GCCTTCCAGATGTCTCAGTCTCCAGCCTCCCTATCTGCATCTGTGGGAGAAACTGTC |
| | ACCATCACATGTCGAGCAAGTGGAAATATTTACAATTATTTAGCATGGTATCAGCAG |
| | AAACAGGGAAAATCTCCTCACCTCCTGGTCTATGATGCAAAAACCTTAGCAGATGGT |
| | GTGCCATCAAGGTTCAGTGGCAGTGGCTCAGGGACACAATATTCTCTCAAGATTAGC |
| | AGCCTGCAGACTGAAGATTCTGGGAATTATTACTGTCAACATTTTTGGAGTCTTCCA |
| | TTCACGTTCGGCTCGGGGACAAAGTTGGAAATAAAAGCGGCCGCA [SEQ ID NO: |
| | 28] |
| scFv | ATGGCCTTACCAGTGACCGCCTTGCTCCTGCCGCTGGCCTTGCTGCTCCACGCCGAG |
| | GTTCAGCTGCAGCAGTCTGGGGCTGAGCTTGTGAGGCCAGGGGCCTTGGTCAAGTTG |
| | TCCTGCAAAACTTCTGGCTTCAACATTAAAGACTACTTTTTACACTGGGTGAGACAG |
| | AGGCCTGACCAGGGCCTGGAGTGGATTGGATGGATTAATCCTGATAATGGTAATACT |
| | GTTTATGACCCGAAGTTTCAGGGCACGGCCAGTTTAACAGCAGACACATCCTCCAAC |
| | ACAGTCTACTTGCAGCTCAGCGGCCTGACATCTGAGGACACTGCCGTCTATTTCTGT |
| | ACTCGGAGGGACTATACTTATGAAAAGGCTGCTCTGGACTACTGGGGTCAGGGAGCC |
| | TCAGTCATCGTCTCCTCAGGTGGAGGTGGATCAGGTGGAGGTGGATCTGGTGGAGGT |
| | GGATCTGCCTTCCAGATGTCTCAGTCTCCAGCCTCCCTATCTGCATCTGTGGGAGAA |
| | ACTGTCACCATCACATGTCGAGCAAGTGGAAATATTTACAATTATTTAGCATGGTAT |
| | CAGCAGAAACAGGGAAAATCTCCTCACCTCCTGGTCTATGATGCAAAAACCTTAGCA |
| | GATGGTGTGCCATCAAGGTTCAGTGGCAGTGGCTCAGGGACACAATATTCTCTCAAG |
| | ATTAGCAGCCTGCAGACTGAAGATTCTGGGAATTATTACTGTCAACATTTTTGGAGT |
| | CTTCCATTCACGTTCGGCTCGGGGACAAAGTTGGAAATAAAAGCGGCCGCA [SEQ |
| | ID NO: 9] |

As used herein, the term “a conservative modification” refers to an amino acid modification that does not significantly affect or alter the binding characteristics of the presently disclosed CAR (e.g., the extracellular antigen-binding domain of the CAR) comprising the amino acid sequence. Conservative modifications can include amino acid substitutions, additions and deletions. Modifications can be introduced into the scFv of the presently disclosed CAR by standard techniques known in the art, such as site-directed mutagenesis and PCR-mediated mutagenesis. Amino acids can be classified into groups according to their physicochemical properties such as charge and polarity. Conservative amino acid substitutions are ones in which the amino acid residue is replaced with an amino acid within the same group. For example, amino acids can be classified by charge: positively-charged amino acids include lysine, arginine, histidine, negatively-charged amino acids include aspartic acid, glutamic acid, neutral charge amino acids include alanine, asparagine, cysteine, glutamine, glycine, isoleucine, leucine, methionine, phenylalanine, proline, serine, threonine, tryptophan, tyrosine, and valine. In addition, amino acids can be classified by polarity: polar amino acids include arginine (basic polar), asparagine, aspartic acid (acidic polar), glutamic acid (acidic polar), glutamine, histidine (basic polar), lysine (basic polar), serine, threonine, and tyrosine; non-polar amino acids include alanine, cysteine, glycine, isoleucine, leucine, methionine, phenylalanine, proline, tryptophan, and valine. Thus, one or more amino acid residues within a CDR region can be replaced with other amino acid residues from the same group and the altered antibody can be tested for retained function (i.e., the functions set forth in (c) through (l) above) using the functional assays described herein. In certain embodiments, no more than one, no more than two, no more than three, no more than four, no more than five residues within a specified sequence or a CDR region are altered.

The V_{H} and/or V_{L} amino acid sequences having at least about 80%, at least about 85%, at least about 90%, or at least about 95% (e.g., about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99%) homology to the specified sequences (e.g., SEQ ID NOs: 7 and/or 8) contain substitutions (e.g., conservative substitutions), insertions, or deletions relative to the specified sequence(s), but retain the ability to bind to an MDA polypeptide (e.g., a human MDA polypeptide (e.g., a human Trp1 polypeptide), and/or a mouse MDA polypeptide (e.g., a mouse Trp1 polypeptide)). In certain embodiments, the extracellular antigen-binding domain specifically binds to an MDA polypeptide (e.g., a human MDA polypeptide (e.g., a human Trp1 polypeptide), and/or a mouse MDA polypeptide (e.g., a mouse Trp1 polypeptide)) with a binding affinity (K_{d}) of about 3×10⁻⁹ or less. In certain embodiments, the extracellular antigen-binding domain binds to an MDA polypeptide (e.g., a human MDA polypeptide (e.g., a human Trp1 polypeptide), or a mouse MDA polypeptide (e.g., a mouse Trp1 polypeptide)) with a binding affinity (K_{d}) of from about 1×10⁻⁹ M to about 3×10⁻⁹ M. In certain embodiments, the extracellular antigen-binding domain binds to an MDA polypeptide (e.g., a human MDA polypeptide (e.g., a human Trp1 polypeptide), and/or a mouse MDA polypeptide (e.g., a mouse Trp1 polypeptide)) with a binding affinity (K_{d}) of from about 1.5×10⁻⁹ M to about 3×10⁻⁹ M. In certain embodiments, a total of 1 to 10 amino acids are substituted, inserted and/or deleted in SEQ ID NOs: 7 and/or 8. In certain embodiments, substitutions, insertions, or deletions occur in regions outside the CDRs (e.g., in the FRs) of the extracellular antigen-binding domain. In certain embodiments, the extracellular antigen-binding domain comprises V_{H} and/or V_{L} sequence selected from the group consisting of SEQ ID NOs: 7 and/or 8, including post-translational modifications of that sequence (SEQ ID NO: 7 and/or 8).

As used herein, the percent homology between two amino acid sequences is equivalent to the percent identity between the two sequences. The percent identity between the two sequences is a function of the number of identical positions shared by the sequences (i.e., % homology=# of identical positions/total # of positions×100), taking into account the number of gaps, and the length of each gap, which need to be introduced for optimal alignment of the two sequences. The comparison of sequences and determination of percent identity between two sequences can be accomplished using a mathematical algorithm.

The percent homology between two amino acid sequences can be determined using the algorithm of E. Meyers and W. Miller (Comput. Appl. Biosci., 4:11-17 (1988)) which has been incorporated into the ALIGN program (version 2.0), using a PAM120 weight residue table, a gap length penalty of 12 and a gap penalty of 4. In addition, the percent homology between two amino acid sequences can be determined using the Needleman and Wunsch (J. Mol. Biol. 48:444-453 (1970)) algorithm which has been incorporated into the GAP program in the GCG software package (available at www.gcg.com), using either a Blossum 62 matrix or a PAM250 matrix, and a gap weight of 16, 14, 12, 10, 8, 6, or 4 and a length weight of 1, 2, 3, 4, 5, or 6.

Additionally or alternatively, the amino acids sequences of the presently disclosed subject matter can further be used as a “query sequence” to perform a search against public databases to, for example, identify related sequences. Such searches can be performed using the XBLAST program (version 2.0) of Altschul, et al. (1990) J. Mol. Biol. 215:403-10. BLAST protein searches can be performed with the XBLAST program, score=50, wordlength=3 to obtain amino acid sequences homologous to the specified sequences (e.g., heavy and light chain variable region sequences of scFv m903, m904, m905, m906, and m900) disclosed herein. To obtain gapped alignments for comparison purposes, Gapped BLAST can be utilized as described in Altschul et al., (1997) Nucleic Acids Res. 25(17):3389-3402. When utilizing BLAST and Gapped BLAST programs, the default parameters of the respective programs (e.g., XBLAST and NBLAST) can be used.

In certain embodiments, the extracellular antigen-binding domain of a presently disclosed CAR cross-competes for binding to an MDA polypeptide (e.g., a human MDA polypeptide (e.g., a human Trp1 polypeptide), and/or a mouse MDA polypeptide (e.g., a mouse Trp1 polypeptide)) with a reference antibody or an antigen-binding portion thereof comprising, e.g., the V_{H} CDR1, CDR2, and CDR3 sequences and/or the V_{L} CDR1, CDR2, and CDR3 sequences described in Table 1. For example, the extracellular antigen-binding domain of a presently disclosed CAR cross-competes for binding to an MDA polypeptide (e.g., a human MDA polypeptide (e.g., a human Trp 1 polypeptide), and/or a mouse MDA polypeptide (e.g., a mouse Trp1 polypeptide)) with a reference antibody or an antigen-binding portion thereof comprising a V_{H} CDR1 comprising amino acids having the sequence set forth in SEQ ID NO: 1; a V_{H} CDR2 comprising amino acids having the sequence set forth in SEQ ID NO: 2; a V_{H} CDR3 comprising amino acids having the sequence set forth in SEQ ID NO: 3; a V_{L} CDR1 comprising amino acids having the sequence set forth in SEQ ID NO: 4; a V_{L} CDR2 comprising amino acids having the sequence set forth in SEQ ID NO: 5; and a V_{L} CDR3 comprising amino acids having the sequence set forth in SEQ ID NO: 6. In certain embodiments, the extracellular antigen-binding domain of a presently disclosed CAR cross-competes for binding to an MDA polypeptide (e.g., a human MDA polypeptide (e.g., a human Trp1 polypeptide), and/or a mouse MDA polypeptide (e.g., a mouse Trp1 polypeptide)) with a reference antibody or an antigen-binding portion thereof comprising, e.g., the V_{H} and V_{L} sequences described in Table 1. For example, the extracellular antigen-binding domain of a presently disclosed CAR cross-competes for binding to an MDA polypeptide (e.g., a human MDA polypeptide (e.g., a human Trp1 polypeptide) and/or a mouse MDA polypeptide (e.g., a mouse Trp1 polypeptide)) with a reference antibody or an antigen-binding portion thereof comprising a V_{H} comprising amino acids having the sequence set forth in SEQ ID NO: 7, and a V_{L} comprising amino acids having the sequence set forth in SEQ ID NO: 8.

In certain embodiments, the extracellular antigen-binding domain binds to the same epitope on an MDA (e.g., a human MDA (e.g., human Trp1) and/or a mouse MDA (e.g., mouse Trp1)) as the reference antibody or antigen-binding portion thereof. For example, the extracellular antigen-binding domain of a presently disclosed CAR binds to the same epitope on an MDA (e.g., a human MDA (e.g., human Trp1) and/or a mouse MDA (e.g., mouse Trp1)) as a reference antibody or an antigen-binding portion thereof comprising, e.g., the V_{H} CDR1, CDR2, and CDR3 sequences and the V_{L} CDR1, CDR2, and CDR3 sequences described in Table 1. For example, the extracellular antigen-binding domain of a presently disclosed CAR binds to the same epitope on an MDA (e.g., a human MDA (e.g., human Trp1, e.g., a human Trp1 polypeptide) and/or a mouse MDA (e.g., mouse Trp1, e.g., a mouse Trp1 polypeptide) as a reference antibody or an antigen-binding portion thereof comprising a V_{H} CDR1 comprising amino acids having the sequence set forth in SEQ ID NO: 1; a V_{H} CDR2 comprising amino acids having the sequence set forth in SEQ ID NO: 2; a V_{H} CDR3 comprising amino acids having the sequence set forth in SEQ ID NO: 3; a V_{L} CDR1 comprising amino acids having the sequence set forth in SEQ ID NO: 4; a V_{L} CDR2 comprising amino acids having the sequence set forth in SEQ ID NO: 5; and a V_{L} CDR3 comprising amino acids having the sequence set forth in SEQ ID NO: 6. In certain embodiments, the extracellular antigen-binding domain of a presently disclosed CAR binds to the same or substantially the same epitope on an MDA (e.g., a human MDA (e.g., human Trp1) and/or a mouse MDA (e.g., mouse Trp1)) as a reference antibody or an antigen-binding portion thereof comprising the V_{H} and V_{L} sequences described in Table 1. For example, the extracellular antigen-binding domain of a presently disclosed CAR binds to the same or substantially the same epitope on an MDA (e.g., a human MDA (e.g., human Trp1) and/or a mouse MDA (e.g., mouse Trp1, e.g., a mouse Trp1 polypeptide) as a reference antibody or an antigen-binding portion thereof comprising a V_{H} comprising amino acids having the sequence set forth in SEQ ID NO: 7, and a V_{L} comprising amino acids having the sequence set forth in SEQ ID NO: 8.

Extracellular antigen-binding domains that cross-compete or compete with the reference antibody or antigen-binding portions thereof for binding to an MDA polypeptide (e.g., a human MDA polypeptide (e.g., a human Trp1 polypeptide) and/or a mouse MDA polypeptide (e.g., a mouse Trp1 polypeptide)) can be identified by using routine methods known in the art, including, but not limited to, ELISAs, radioimmunoassays (RIAs), Biacore, flow cytometry, Western blotting, and any other suitable quantitative or qualitative antibody-binding assays. Competition ELISA is described in Morris, “Epitope Mapping of Protein Antigens by Competition ELISA”, The Protein Protocols Handbook (1996), pp 595-600, edited by J. Walker, which is incorporated by reference in its entirety. In certain embodiments, the antibody-binding assay comprises measuring an initial binding of a reference antibody or an antigen-binding portion thereof to an MDA polypeptide (e.g., a human MDA polypeptide (e.g., a human Trp1 polypeptide) and/or a mouse MDA polypeptide (e.g., a mouse Trp1 polypeptide)), admixing the reference antibody with a test extracellular antigen-binding domain, measuring a second binding of the reference antibody or antigen-binding portion thereof to the MDA polypeptide in the presence of the test extracellular antigen-binding domain, and comparing the initial binding with the second binding of the reference antibody, wherein a decreased second binding of the reference antibody or antigen-binding portion thereof to the MDA polypeptide in comparison to the initial binding indicates that the test extracellular antigen-binding domain cross-competes with the reference antibody or antigen-binding portion thereof for binding to the MDA polypeptide, e.g., one that recognizes the same or substantially the same epitope, an overlapping epitope, or an adjacent epitope. In certain embodiments, the reference antibody or antigen-binding portion thereof is labeled, e.g., with a fluorochrome, biotin, or peroxidase. In certain embodiments, the MDA polypeptide is expressed in cells, e.g., in a flow cytometry test. In certain embodiments, the MDA polypeptide is immobilized onto a surface, including a Biacore ship (e.g., in a Biacore test), or other media suitable for surface plasmon resonance analysis. The binding of the reference antibody or antigen-binding portion thereof in the presence of a completely irrelevant antibody (that does not bind to the MDA polypeptide) can serve as the control high value. The control low value can be obtained by incubating a labeled reference antibody with an unlabeled reference antibody, where competition and reduced binding of the labeled reference antibody would occur. In certain embodiments, a test extracellular antigen-binding domain that reduces the binding of the reference antibody or antigen-binding portion thereof to an MDA polypeptide by at least about 20%, at least about 30%, at least about 40%, at least about 50%, at least about 60%, at least about 70%, at least about 80%, at least about 90%, or at least about 95% is considered to be an extracellular antigen-binding domain that cross-competes with the reference antibody or antigen-binding portion thereof for binding to the MDA polypeptide. In certain embodiments, the assays are performed at room temperature.

It is well known in the art that the CDR3 domain, independently from the CDR1 and/or CDR2 domain(s), alone can determine the binding specificity of an antibody or an antigen-binding portion thereof, for a cognate antigen and that multiple antibodies can predictably be generated having the same binding specificity based on a common CDR3 sequence. See, for example, Klimka et al., British J. of Cancer 83(2):252-260 (2000) (describing the production of a humanized anti-CD30 antibody using only the heavy chain variable domain CDR3 of murine anti-CD30 antibody Ki-4); Beiboer et al., J. Mol. Bioi. 296:833-849 (2000) (describing recombinant epithelial glycoprotein-2 (EGP-2) antibodies using only the heavy chain CDR3 sequence of the parental murine MOC-31 anti-EGP-2 antibody); Rader et al., Proc. Natl. Acad Sci. US.A. 95:8910-8915 (1998) (describing a panel of humanized anti-integrin αᵥβ3 antibodies using a heavy and light chain variable CDR3 domain of a murine anti-integrin αᵥβ3 antibody LM609 wherein each member antibody comprises a distinct sequence outside the CDR3 domain and capable of binding the same epitope as the parent muring antibody with affinities as high or higher than the parent murine antibody); Barbas et al., J. Am. Chem. Soc. 116:2161-2162 (1994) (disclosing that the CDR3 domain provides the most significant contribution to antigen binding); Barbas et al., Proc. Natl. Acad Sci. US.A. 92:2529-2533 (1995) (describing the grafting of heavy chain CDR3 sequences of three Fabs (SI-1, SI-40, and SI-32) against human placental DNA onto the heavy chain of an anti-tetanus toxoid Fab thereby replacing the existing heavy chain CDR3 and demonstrating that the CDR3 domain alone conferred binding specificity); and Ditzel et ai., J. Immunol. 157:739-749 (1996) (describing grafting studies wherein transfer of only the heavy chain CDR3 of a parent polyspecific Fab LNA3 to a heavy chain of a monospecific IgG tetanus toxoid-binding Fab p313 antibody was sufficient to retain binding specificity of the parent Fab). Each of these references is hereby incorporated by reference in its entirety. In certain embodiments, the extracellular antigen-binding domain comprises a heavy chain variable region CDR3 comprising amino acids having the sequence set forth in SEQ ID NO: 3 or a conservative modification thereof, and/or a light chain variable region CDR3 comprising amino acids having the sequence set forth in SEQ ID NO: 6 or a conservative modification thereof. The extracellular antigen-binding domain can comprise a heavy chain variable region CDR2 comprising amino acids having the sequence set forth in SEQ ID NO: 2 or a conservative modification thereof, and a light chain variable region CDR2 comprising amino acids having the sequence set forth in SEQ ID NO: 5 or a conservative modification thereof. The extracellular antigen-binding domain can further comprise a heavy chain variable region CDR1 comprising amino acids having the sequence set forth in SEQ ID NO: 1 or a conservative modification thereof, and a light chain variable region CDR1 comprising amino acids having the sequence set forth in SEQ ID NO: 4 or a conservative modification thereof.

In certain embodiments, the extracellular antigen-binding domain comprises a V_{H} CDR1 comprising amino acids having the sequence set forth in SEQ ID NO: 1, a V_{H} CDR2 comprising amino acids having the sequence set forth in SEQ ID NO: 2, a V_{H} CDR3 comprising amino acids having the sequence set forth in SEQ ID NO: 3, a V_{L} CDR1 comprising amino acids having the sequence set forth in SEQ ID NO: 4, a V_{L} CDR2 comprising amino acids having the sequence set forth in SEQ ID NO: 5, and a V_{L} CDR3 comprising amino acids having the sequence set forth in SEQ ID NO: 6.

In certain embodiments, an extracellular antigen-binding domain of a presently disclosed CAR can comprise a linker connecting the heavy chain variable region and light chain variable region of the extracellular antigen-binding domain. As used herein, the term “linker” refers to a functional group (e.g., chemical or polypeptide) that covalently attaches two or more polypeptides or nucleic acids so that they are connected to one another. As used herein, a “peptide linker” refers to one or more amino acids used to couple two proteins together (e.g., to couple V_{H} and V_{L} domains). In one non-limiting example, the linker comprises amino acids having the sequence set forth in SEQ ID NO: 11. In one embodiment, the nucleotide sequence encoding the amino acid sequence of SEQ ID NO: 11 is set forth in SEQ ID NO: 26.

In addition, the extracellular antigen-binding domain can comprise a leader or a signal peptide that directs the nascent protein into the endoplasmic reticulum. Signal peptide or leader can be essential if the CAR is to be glycosylated and anchored in the cell membrane. The signal sequence or leader can be a peptide sequence (about 5, about 10, about 15, about 20, about 25, or about 30 amino acids long) present at the N-terminus of newly synthesized proteins that directs their entry to the secretory pathway. In non-limiting examples, the signal peptide is covalently joined to the 5′ terminus of the extracellular antigen-binding domain. In certain embodiments, the signal peptide comprises amino acids having the sequence set forth in SEQ ID NO: 12 as provided below.

[SEQ ID NO: 12]
MALPVTALLLPLALLLHA

The nucleotide sequence encoding the amino acid sequence of SEQ ID NO: 12 is set forth in SEQ ID NO: 29, which is provided below:

[SEQ ID NO: 29]
ATGGCCTTACCAGTGACCGCCTTGCTCCGBCCGCTGGCCTTGCTGCTCC
ACGCC

### Transmembrane Domain of a CAR

In certain non-limiting embodiments, the transmembrane domain of the CAR comprises a hydrophobic alpha helix that spans at least a portion of the membrane. Different transmembrane domains result in different receptor stability. After antigen recognition, receptors cluster and a signal is transmitted to the cell. In accordance with the presently disclosed subject matter, the transmembrane domain of the CAR can comprise a CD8 polypeptide, a CD28 polypeptide, a CD3zeta polypeptide, a CD4 polypeptide, a 4-1BB polypeptide, an OX40 polypeptide, an ICOS polypeptide, a CTLA-4 polypeptide, a PD-1 polypeptide, a LAG-3 polypeptide, a 2B4 polypeptide, a BTLA polypeptide, a synthetic peptide (not based on a protein associated with the immune response), or a combination thereof.

In certain embodiments, the transmembrane domain comprises a CD8 polypeptide. In certain embodiments, the CD8 polypeptide has an amino acid sequence that is at least about 85%, about 90%, about 95%, about 96%, about 97%, about 98%, about 99% or about 100% homologous to the sequence having a NCBI Reference No: NP_001139345.1 (SEQ ID NO: 13) (homology herein may be determined using standard software such as BLAST or FASTA) as provided below, or fragments thereof, and/or may optionally comprise up to one or up to two or up to three conservative amino acid substitutions. In certain embodiments, the CD8 polypeptide can have an amino acid sequence that is a consecutive portion of SEQ ID NO: 13 which is at least 20, or at least 30, or at least 40, or at least 50, and up to 235 amino acids in length. Alternatively or additionally, in non-limiting various embodiments, the CD8 polypeptide comprises or has an amino acid sequence of amino acids 1 to 235, 1 to 50, 50 to 100, 100 to 150, 150 to 200, or 200 to 235 of SEQ ID NO: 13. In certain embodiments, the CAR of the presently disclosed comprises a transmembrane domain comprising a CD8 polypeptide that comprises an amino acid sequence of amino acids 137 to 209 of SEQ ID NO: 13.

[SEQ ID NO: 13]
MALPVTALLLPLALLLHAARPSQFRVSPLDRTWNLGETVELKCQVLLS
NPTSGCSWLFQPRGAAASPTFLLYLSQNKPKAAEGLDTQRFSGKRLGD
TFVLTLSDFRRENEGYYFCSALSNSIMYFSHFVPVFLPAKPTTTPAPR
PPTPAPTIASQPLSLRPEACRPAAGGAVHTRGLDFACDIYIWAPLAGT
CGVLLLSLVITLYCNHRNRRRVCKCPRPVVKSGDKPSLSARYV

In certain embodiments, the CD8 polypeptide has an amino acid sequence that is at least about 85%, about 90%, about 95%, about 96%, about 97%, about 98%, about 99% or about 100% homologous to the sequence having a NCBI Reference No: AAA92533.1 (SEQ ID NO: 30) (homology herein may be determined using standard software such as BLAST or FASTA) as provided below, or fragments thereof, and/or may optionally comprise up to one or up to two or up to three conservative amino acid substitutions. In certain embodiments, the CD8 polypeptide can have an amino acid sequence that is a consecutive portion of SEQ ID NO: 30 which is at least about 20, or at least about 30, or at least about 40, or at least about 50, or at least about 60, or at least about 70, or at least about 100, or at least about 200, and up to 247 amino acids in length. Alternatively or additionally, in non-limiting various embodiments, the CD8 polypeptide comprises or has an amino acid sequence of amino acids 1 to 247, 1 to 50, 50 to 100, 100 to 150, 150 to 200, 151 to 219, or 200 to 247 of SEQ ID NO: 30. In certain embodiments, the CAR of the presently disclosed comprises a transmembrane domain comprising a CD8 polypeptide that comprises an amino acid sequence of amino acids 151 to 219 of SEQ ID NO: 30.

```
[SEQ ID NO: 30]
    1 MASPLTRELS LNLLLMGESI ILGSGEAKPQ APELRIFPKK MDAELGQKVD LVCEVLGSVS
  61 QGCSWLFQNS SSKLPQPTFV VYMASSHNKI TWDEKLNSSK LFSAVRDTNN KYVLTLNKFS
121 KENEGYYFCS VISNSVMYFS SVVPVLQKVN STTTKPVLRT PSPVHPTGTS QPQRPEDCRP
181 RGSVKGTGLD FACDIYIWAP LAGICVAPLL SLIITLICYH RSRKRVCKCP RPLVRQEGKP
241 RPSEKIV
```

In certain embodiments, the CD8 polypeptide comprises or has the amino acid sequence set forth in SEQ ID NO: 32, which is provided below:

[SEQ ID NO: 32]
STTTKPVLRTPSPVHPTGTSQPQRPEDCRPRGSVKGTGLDFACDIYIW
APLAGICVALLLSLIITLICY

In accordance with the presently disclosed subject matter, a “CD8 nucleic acid molecule” refers to a polynucleotide encoding a CD8 polypeptide.

In certain embodiments, the CD8 nucleic acid molecule encoding the CD8 polypeptide comprised in the transmembrane domain of the presently disclosed CAR (SEQ ID NO: 32) comprises nucleic acids having the sequence set forth in SEQ ID NO: 33 as provided below.

[SEQ ID NO: 33]
TCTACTACTACCAAGCCAGTGCTGCGAACTCCCTCACCTGTGCACCCT
ACCGGGACATCTCAGCCCCAGAGACCAGAAGATTGTCGGCCCCGTGGC
TCAGTGAAGGGGACCGGATTGGACTTCGCCTGTGATATTTACATCTGG
GCACCCTTGGCCGGAATCTGCGTGGCCCTTCTGCTGTCCTTGATCATC
ACTCTCATCTGCTAC

In certain embodiments, the transmembrane domain of a presently disclosed CAR comprises a CD28 polypeptide. The CD28 polypeptide can have an amino acid sequence that is at least about 85%, about 90%, about 95%, about 96%, about 97%, about 98%, about 99% or 100% homologous to the sequence having a NCBI Reference No: P10747 or NP_006130 (SEQ ID NO:14), or fragments thereof, and/or may optionally comprise up to one or up to two or up to three conservative amino acid substitutions. In certain non-limiting embodiments, the CD28 polypeptide can have an amino acid sequence that is a consecutive portion of SEQ ID NO: 14 which is at least 20, or at least 30, or at least 40, or at least 50, and up to 220 amino acids in length. Alternatively or additionally, in various non-limiting embodiments, the CD28 polypeptide has an amino acid sequence of amino acids 1 to 220, 1 to 50, 50 to 100, 100 to 150, 114 to 220, 150 to 200, or 200 to 220 of SEQ ID NO: 14.

SEQ ID NO: 14 is provided below:

```
[SEQ ID NO: 14]
    1 MLRLLLALNL FPSIQVTGNK ILVKQSPMLV AYDNAVNLSC KYSYNLFSRE FRASLHKGLD
  61 SAVEVCVVYG NYSQQLQVYS KTGFNCDGKL GNESVTFYLQ NLYVNQTDIY FCKIEVMYPP
121 PYLDNEKSNG TIIHVKGKHL CPSPLFPGPS KPFWVLVVVG GVLACYSLLV TVAFIIFWVR
181 SKRSRLLHSD YMNMTPRRPG PTRKHYQPYA PPRDFAAYRS
```

In accordance with the presently disclosed subject matter, a “CD28 nucleic acid molecule” refers to a polynucleotide encoding a CD28 polypeptide.

In certain non-limiting embodiments, a CAR can also comprise a spacer region that links the extracellular antigen-binding domain to the transmembrane domain. The spacer region can be flexible enough to allow the antigen binding domain to orient in different directions to facilitate antigen recognition. The spacer region can be the hinge region from IgG1, or the CH2CH3 region of immunoglobulin and portions of CD3.

### Intracellular Domain of a CAR

In certain non-limiting embodiments, an intracellular domain of the CAR can comprise a CD3zeta polypeptide, which can activate or stimulate a cell (e.g., a cell of the lymphoid lineage, e.g., a T cell). CD3zeta comprises 3 ITAMs, and transmits an activation signal to the cell (e.g., a cell of the lymphoid lineage, e.g., a T cell) after antigen is bound. In certain embodiments, the CD3zeta polypeptide has an amino acid sequence that is at least about 85%, about 90%, about 95%, about 96%, about 97%, about 98%, about 99% or about 100% homologous to the sequence having a NCBI Reference No: NP_932170 (SEQ ID NO: 15), or fragments thereof, and/or may optionally comprise up to one or up to two or up to three conservative amino acid substitutions. In certain non-limiting embodiments, the CD3zeta polypeptide can have an amino acid sequence that is a consecutive portion of SEQ ID NO: 15 which is at least 20, or at least 30, or at least 40, or at least 50, and up to 164 amino acids in length. Alternatively or additionally, in non-limiting various embodiments, the CD3zeta polypeptide has an amino acid sequence of amino acids 1 to 164, 1 to 50, 50 to 100, 100 to 150, or 150 to 164 of SEQ ID NO: 15. In certain embodiments, the CD3zeta polypeptide comprises or has an amino acid sequence of amino acids 52 to 164 of SEQ ID NO: 15.

SEQ ID NO: 15 is provided below:

[SEQ ID NO: 15]

| | |
|---|---|
| 1 | MKWKALFTAA ILQAQLPITE AQSFGLLDPK LCYLLDGILF |
| | IYGVILTALF LRVKFSRSAD |
| 61 | APAYQQGQNQ LYNELNLGRR EEYDVLDKRR GRDPEMGGKP |
| | QRRKNPQEGL YNELQKDKMA |
| 121 | EAYSEIGMKG ERRRGKGHDG LYQGLSTATK DTYDALHMQA |
| | LPPR |

In certain embodiments, the CD3zeta polypeptide has an amino acid sequence that is at least about 85%, about 90%, about 95%, about 96%, about 97%, about 98%, about 99% or about 100% homologous to the sequence having a NCBI Reference No: NP_001106864.2 (SEQ ID NO: 16), or fragments thereof, and/or may optionally comprise up to one or up to two or up to three conservative amino acid substitutions. In certain non-limiting embodiments, the CD3zeta polypeptide can have an amino acid sequence that is a consecutive portion of SEQ ID NO: 16 which is at least about 20, or at least about 30, or at least about 40, or at least about 50, or at least about 90, or at least about 100, and up to 188 amino acids in length. Alternatively or additionally, in non-limiting various embodiments, the CD3zeta polypeptide has an amino acid sequence of amino acids 1 to 164, 1 to 50, 50 to 100, 52 to 142, 100 to 150, or 150 to 188 of SEQ ID NO: 16. In certain embodiments, the CD3zeta polypeptide comprises or has an amino acid sequence of amino acids 52 to 142 of SEQ ID NO: 16.

SEQ ID NO: 16 is provided below:

[SEQ ID NO: 16]

| | |
|---|---|
| 1 | MKWKVSVLAC ILHVRFPGAE AQSFGLLDPK LCYLLDGILF |
| | IYGVIITALY LRAKFSRSAE |
| 61 | TAANLQDPNQ LYNELNLGRR EEYDVLEKKR ARDPEMGGKQ |
| | RRRNPQEGVY NALQKDKMAE |
| 121 | AYSEIGTKGE RRRGKGHDGL YQDSHFQAVQ FGNRREREGS |
| | ELTRTLGLRA RPKACRHKKP |
| 181 | LSLPAAVS |

In certain embodiments, the CD3zeta polypeptide comprises or has the amino acid sequence set forth in SEQ ID NO: 34, which is provided below:

[SEQ ID NO: 34]
RAKFSRSAETAANLQDPNQLYNELNLGRREEYDVLEKKRARDPEMGGKQQ
RRRNPQEGVYNALQKDKMAEAYSEIGTKGERRRGKGHDGLYQGLSTATKD
TYDALHMQTLAPR

In accordance with the presently disclosed subject matter, a “CD3zeta nucleic acid molecule” refers to a polynucleotide encoding a CD3zeta polypeptide. In certain embodiments, the CD3zeta nucleic acid molecule encoding the CD3zeta polypeptide comprised in the intracellular domain of a presently disclosed CAR (SEQ ID NO: 34) comprises the nucleotide sequence set forth in SEQ ID NO: 31 as provided below.

[SEQ ID NO: 31]
AGAGCAAAATTCAGCAGGAGTGCAGAGACTGCTGCCAACCTGCAGGACCC
CAACCAGCTCTACAATGAGCTCAATCTAGGGCGAAGAGAGGAATATGACG
TCTTGGAGAAGAAGCGGGCTCGGGATCCAGAGATGGGAGGCAAACAGCAG
AGGAGGAGGAACCCCCAGGAAGGCGTATACAATGCACTGCAGAAAGACAA
GATGGCAGAAGCCTACAGTGAGATCGGCACAAAAGGCGAGAGGCGGAGAG
GCAAGGGGCACGATGGCCTTTACCAGGGTCTCAGCACTGCCACCAAGGAC
ACCTATGATGCCCTGCATATGCAGACCCTGGCCCCTCGCTAA

In certain non-limiting embodiments, an intracellular domain of the CAR further comprises at least one signaling region. The at least one signaling region can include a CD28 polypeptide, a 4-1BB polypeptide, an OX40 polypeptide, an ICOS polypeptide, a DAP-10 polypeptide, a PD-1 polypeptide, a CTLA-4 polypeptide, a LAG-3 polypeptide, a 2B4 polypeptide, a BTLA polypeptide, a synthetic peptide (not based on a protein associated with the immune response), or a combination thereof.

In certain embodiments, the signaling region is a co-stimulatory signaling region. In certain embodiments, the co-stimulatory region comprises at least one co-stimulatory molecule, which can provide optimal lymphocyte activation. As used herein, “co-stimulatory molecules” refer to cell surface molecules other than antigen receptors or their ligands that are required for an efficient response of lymphocytes to antigen. The at least one co-stimulatory signaling region can include a CD28 polypeptide, a 4-1BB polypeptide, an OX40 polypeptide, an ICOS polypeptide, a DAP-10 polypeptide, or a combination thereof. The co-stimulatory molecule can bind to a co-stimulatory ligand, which is a protein expressed on cell surface that upon binding to its receptor produces a co-stimulatory response, i.e., an intracellular response that effects the stimulation provided when an antigen binds to its CAR molecule. Co-stimulatory ligands, include, but are not limited to CD80, CD86, CD70, OX40L, 4-1BBL, CD48, TNFRSF14, and PD-L1. As one example, a 4-1BB ligand (i.e., 4-1BBL) may bind to 4-1BB (also known as “CD137”) for providing an intracellular signal that in combination with a CAR signal induces an effector cell function of the CAR⁺ T cell. CARs comprising an intracellular domain that comprises a co-stimulatory signaling region comprising 4-1BB, ICOS or DAP-10 are disclosed in U.S. Pat. No. 7,446,190 (e.g., the nucleotide sequence encoding 4-1BB is set forth in SEQ ID NO:15, the nucleotide sequence encoding ICOS is set forth in SEQ ID NO: 16, and the nucleotide sequence encoding DAP-10 is set forth in SEQ ID NO:17 in U.S. Pat. No. 7,446,190), which is herein incorporated by reference in its entirety. In certain embodiments, the intracellular domain of the CAR comprises a co-stimulatory signaling region that comprises a CD28 polypeptide.

In certain embodiments, the CD28 polypeptide has an amino acid sequence that is at least about 85%, about 90%, about 95%, about 96%, about 97%, about 98%, about 99% or 100% homologous to the sequence having a NCBI Reference No: P10747 or NP_006130 (SEQ ID NO: 14), or fragments thereof, and/or may optionally comprise up to one or up to two or up to three conservative amino acid substitutions. In non-limiting certain embodiments, the CD28 polypeptide has an amino acid sequence that is a consecutive portion of SEQ ID NO: 14 which is at least 20, or at least 30, or at least 40, or at least 50, and up to 220 amino acids in length. Alternatively or additionally, in non-limiting various embodiments, the CD28 polypeptide has an amino acid sequence of amino acids 1 to 220, 1 to 50, 50 to 100, 100 to 150, 114 to 220, 150 to 200, or 200 to 220 of SEQ ID NO: 14.

In certain embodiments, the CD28 polypeptide has an amino acid sequence that is at least about 85%, about 90%, about 95%, about 96%, about 97%, about 98%, about 99% or 100% homologous to the sequence having a NCBI Reference No: NP_031668.3 (SEQ ID NO: 35), or fragments thereof, and/or may optionally comprise up to one or up to two or up to three conservative amino acid substitutions. In non-limiting certain embodiments, the CD28 polypeptide has an amino acid sequence that is a consecutive portion of SEQ ID NO: 35 which is at least about 20, or at least about 30, or at least about 40, or at least about 50, and up to 218 amino acids in length. Alternatively or additionally, in non-limiting various embodiments, the CD28 polypeptide has an amino acid sequence of amino acids 1 to 218, 1 to 50, 50 to 100, 100 to 150, 114 to 220, 150 to 200, 178 to 218, or 200 to 220 of SEQ ID NO: 35. In certain embodiments, the co-stimulatory signaling region of a presently disclosed CAR comprises a CD28 polypeptide that comprises or has the amino acids 178 to 218 of SEQ ID NO: 35.

SEQ ID NO: 35 is provided below:

[SEQ ID NO: 35]

| | |
|---|---|
| 1 | MTLRLLFLAL NFFSVQVTEN KILVKQSPLL VVDSNEVSLS |
| | CRYSYNLLAK EFRASLYKGV |
| 61 | NSDVEVCVGN GNFTYQPQFR SNAEFNCDGD FDNETVTFRL |
| | WNLHVNHTDI YFCKIEFMYP |
| 121 | PPYLDNERSN GTIIHIKEKH LCHTQSSPKL FWALVVVAGV |
| | LFCYGLLVTV ALCVIWTNSR |
| 181 | RNRLLQSDYM NMTPRRPGLT RKPYQPYAPA RDFAAYRP |

In accordance with the presently disclosed subject matter, a “CD28 nucleic acid molecule” refers to a polynucleotide encoding a CD28 polypeptide. In certain embodiments, a CD28 nucleic acid molecule that encodes a CD28 polypeptide comprised in the co-stimulatory signaling region of a presently disclosed CAR (e.g., amino acids 178 to 218 of SEQ ID NO: 35) comprises or has a nucleotide sequence set forth in SEQ ID NO: 36, which is provided below.

[SEQ ID NO: 36]
AATAGTAGAAGGAACAGACTCCTTCAAAGTGACTACATGAACATGACTCC
CCGGAGGCCTGGGCTCACTCGAAAGCCTTACCAGCCCTACGCCCCTGCCA
GAGACTTTGCAGCGTACCGCCCC

In certain embodiments, the intracellular domain of the CAR comprises a co-stimulatory signaling region that comprises two co-stimulatory molecules: CD28 and 4-1BB or CD28 and OX40.

4-1BB can act as a tumor necrosis factor (TNF) ligand and have stimulatory activity. The 4-1BB polypeptide can have an amino acid sequence that is at least about 85%, about 90%, about 95%, about 96%, about 97%, about 98%, about 99% or 100% homologous to the sequence having a NCBI Reference No: P41273 or NP_001552 (SEQ ID NO: 17) or fragments thereof, and/or may optionally comprise up to one or up to two or up to three conservative amino acid substitutions.

SEQ ID NO: 17 is provided below:

[SEQ ID NO: 17]

| | |
|---|---|
| 1 | MGNSCYNIVA TLLLVLNFER TRSLQDPCSN CPAGTFCDNN |
| | RNQICSPCPP NSFSSAGGQR |
| 61 | TCDICRQCKG VFRTRKECSS TSNAECDCTP GFHCLGAGCS |
| | MCEQDCKQGQ ELTKKGCKDC |
| 121 | CFGTFNDQKR GICRPWTNCS LDGKSVLVNG TKERDVVCGP |
| | SPADLSPGAS SVTPPAPARE |
| 181 | PGHSPQIISF FLALTSTALL FLLFFLTLRF SVVKRGRKKL |
| | LYIFKQPFMR PVQTTQEEDG |
| 241 | CSCRFPEEEE GGCEL |

In accordance with the presently disclosed subject matter, a “4-1BB nucleic acid molecule” refers to a polynucleotide encoding a 4-1BB polypeptide.

An OX40 polypeptide can have an amino acid sequence that is at least about 85%, about 90%, about 95%, about 96%, about 97%, about 98%, about 99% or 100% homologous to the sequence having a NCBI Reference No: P43489 or NP_003318 (SEQ ID NO: 18), or fragments thereof, and/or may optionally comprise up to one or up to two or up to three conservative amino acid substitutions.

SEQ ID NO: 18 is provided below:

[SEQ ID NO: 18]

| | |
|---|---|
| 1 | MCVGARRLGR GPCAALLLLG LGLSTVTGLH CVGDTYPSND |
| | RCCHECRPGN GMVSRCSRSQ |
| 61 | NTVCRPCGPG FYNDVVSSKP CKPCTWCNLR SGSERKQLCT |
| | ATQDTVCRCR AGTQPLDSYK |
| 121 | PGVDCAPCPP GHFSPGDNQA CKPWTNCTLA GKHTLQPASN |
| | SSDAICEDRD PPATQPQETQ |
| 181 | GPPARPITVQ PTEAWPRTSQ GPSTRPVEVP GGRAVAAILG |
| | LGLVLGLLGP LAILLALYLL |
| 241 | RRDQRLPPDA HKPPGGGSFR TPIQEEQADA HSTLAKI |

In accordance with the presently disclosed subject matter, an “OX40 nucleic acid molecule” refers to a polynucleotide encoding an OX40 polypeptide.

An ICOS polypeptide can have an amino acid sequence that is at least about 85%, about 90%, about 95%, about 96%, about 97%, about 98%, about 99% or 100% homologous to the sequence having a NCBI Reference No: NP_036224 (SEQ ID NO: 19) or fragments thereof, and/or may optionally comprise up to one or up to two or up to three conservative amino acid substitutions.

SEQ ID NO: 19 is provided below:

[SEQ ID NO: 19]

| | |
|---|---|
| 1 | MKSGLWYFFL FCLRIKVLTG EINGSANYEM FIFHNGGVQI |
| | LCKYPDIVQQ FKMQLLKGGQ |
| 61 | ILCDLIKTKG SGNTVSIKSL KFCHSQLSNN SVSFFLYNLD |
| | HSHANYYFCN LSIFDPPPFK |
| 121 | VTLIGGYLHI YESQLCCQLK FWLPIGCAAF VVVCILGCIL |
| | ICWLTKKKYS SSVHDPNGEY |
| 181 | MFMRAVNTAK KSRLTDVTL |

In accordance with the presently disclosed subject matter, an “ICOS nucleic acid molecule” refers to a polynucleotide encoding an ICOS polypeptide.

CTLA-4 is an inhibitory receptor expressed by activated T cells, which when engaged by its corresponding ligands (CD80 and CD86; B7-1 and B7-2, respectively), mediates activated T cell inhibition or anergy. In both preclinical and clinical studies, CTLA-4 blockade by systemic antibody infusion, enhanced the endogenous anti-tumor response albeit, in the clinical setting, with significant unforeseen toxicities.

CTLA-4 contains an extracellular V domain, a transmembrane domain, and a cytoplasmic tail. Alternate splice variants, encoding different isoforms, have been characterized. The membrane-bound isoform functions as a homodimer interconnected by a disulfide bond, while the soluble isoform functions as a monomer. The intracellular domain is similar to that of CD28, in that it has no intrinsic catalytic activity and contains one YVKM motif able to bind PI3K, PP2A and SHP-2 and one proline-rich motif able to bind SH3 containing proteins. One role of CTLA-4 in inhibiting T cell responses seem to be directly via SHP-2 and PP2A dephosphorylation of TCR-proximal signaling proteins such as CD3 and LAT. CTLA-4 can also affect signaling indirectly via competing with CD28 for CD80/86 binding. CTLA-4 has also been shown to bind and/or interact with PI3K, CD80, AP2M1, and PPP2R5A.

In accordance with the presently disclosed subject matter, a CTLA-4 polypeptide can have an amino acid sequence that is at least about 85%, about 90%, about 95%, about 96%, about 97%, about 98%, about 99% or about 100% homologous to UniProtKB/Swiss-Prot Ref. No.: P16410.3 (SEQ ID NO: 20) (homology herein may be determined using standard software such as BLAST or FASTA) or fragments thereof, and/or may optionally comprise up to one or up to two or up to three conservative amino acid substitutions.

SEQ ID NO: 20 is provided below:

[SEQ ID NO: 20]

| | |
|---|---|
| 1 | MACLGFQRHK AQLNLATRTW PCTLLFFLLF IPVFCKAMHV |
| | AQPAVVLASS RGIASFVCEY |
| 61 | ASPGKATEVR VTVLRQADSQ VTEVCAATYM MGNELTFLDD |
| | SICTGTSSGN QVNLTIQGLR |
| 121 | AMDTGLYICK VELMYPPPYY LGIGNGTQIY VIDPEPCPDS |
| | DFLLWILAAV SSGLFFYSFL |
| 181 | LTAVSLSKML KKRSPLTTGV YVKMPPTEPE CEKQFQPYFI |
| | PIN |

In accordance with the presently disclosed subject matter, a “CTLA-4 nucleic acid molecule” refers to a polynucleotide encoding a CTLA-4 polypeptide.

PD-1 is a negative immune regulator of activated T cells upon engagement with its corresponding ligands PD-L1 and PD-L2 expressed on endogenous macrophages and dendritic cells. PD-1 is a type I membrane protein of 268 amino acids. PD-1 has two ligands, PD-L1 and PD-L2, which are members of the B7 family. The protein's structure comprises an extracellular IgV domain followed by a transmembrane region and an intracellular tail. The intracellular tail contains two phosphorylation sites located in an immunoreceptor tyrosine-based inhibitory motif and an immunoreceptor tyrosine-based switch motif, that PD-1 negatively regulates TCR signals. SHP-I and SHP-2 phosphatases bind to the cytoplasmic tail of PD-1 upon ligand binding. Upregulation of PD-L1 is one mechanism tumor cells may evade the host immune system. In pre-clinical and clinical trials, PD-1 blockade by antagonistic antibodies induced anti-tumor responses mediated through the host endogenous immune system.

In accordance with the presently disclosed subject matter, a PD-1 polypeptide can have an amino acid sequence that is at least about 85%, about 90%, about 95%, about 96%, about 97%, about 98%, about 99% or about 100% homologous to NCBI Reference No: NP_005009.2 (SEQ ID NO: 21) or fragments thereof, and/or may optionally comprise up to one or up to two or up to three conservative amino acid substitutions.

SEQ ID NO: 21 is provided below:

[SEQ ID NO: 21]

| | |
|---|---|
| 1 | MQIPQAPWPV VWAVLQLGWR PGWFLDSPDR PWNPPTFSPA |
| | LLVVTEGDNA TFTCSFSNTS |
| 61 | ESFVLNWYRM SPSNQTDKLA AFPEDRSQPG QDCRFRVTQL |
| | PNGRDFHMSV VRARRNDSGT |
| 121 | YLCGAISLAP KAQIKESLRA ELRVTERRAE VPTAHPSPSP |
| | RPAGQFQTLV VGVVGGLLGS |
| 181 | LVLLVWVLAV ICSRAARGTI GARRTGQPLK EDPSAVPVFS |
| | VDYGELDFQW REKTPEPPVP |
| 241 | CVPEQTEYAT IVFPSGMGTS SPARRGSADG PRSAQPLRPE |
| | DGHCSWPL |

In accordance with the presently disclosed subject matter, a “PD-1 nucleic acid molecule” refers to a polynucleotide encoding a PD-1 polypeptide.

Lymphocyte-activation protein 3 (LAG-3) is a negative immune regulator of immune cells. LAG-3 belongs to the immunoglobulin (lg) superfamily and contains 4 extracellular Ig-like domains. The LAG3 gene contains 8 exons. The sequence data, exon/intron organization, and chromosomal localization all indicate a close relationship of LAG3 to CD4. LAG3 has also been designated CD223 (cluster of differentiation 223).

In accordance with the presently disclosed subject matter, a LAG-3 polypeptide can have an amino acid sequence that is at least about 85%, about 90%, about 95%, about 96%, about 97%, about 98%, about 99% or about 100% homologous to UniProtKB/Swiss-Prot Ref. No.: P18627.5 (SEQ ID NO: 22) or fragments thereof, and/or may optionally comprise up to one or up to two or up to three conservative amino acid substitutions.

SEQ ID NO: 22 is provided below:

[SEQ ID NO: 22]

| | |
|---|---|
| 1 | MWEAQFLGLL FLQPLWVAPV KPLQPGAEVP VVWAQEGAPA |
| | QLPCSPTIPL QDLSLLRRAG |
| 61 | VTWQHQPDSG PPAAAPGHPL APGPHPAAPS SWGPRPRRYT |
| | VLSVGPGGLR SGRLPLQPRV |
| 121 | QLDERGRQRG DFSLWLRPAR RADAGEYRAA VHLRDRALSC |
| | RLRLRLGQAS MTASPPGSLR |
| 181 | ASDWVILNCS FSRPDRPASV HWFRNRGQGR VPVRESPHHH |
| | LAESFLFLPQ VSPMDSGPWG |
| 241 | CILTYRDGFN VSIMYNLTVL GLEPPTPLTV YAGAGSRVGL |
| | PCRLPAGVGT RSFLTAKWTP |
| 301 | PGGGPDLLVT GDNGDFTLRL EDVSQAQAGT YTCHIHLQEQ |
| | QLNATVTLAI ITVTPKSFGS |
| 361 | PGSLGKLLCE VTPVSGQERF VWSSLDTPSQ RSFSGPWLEA |
| | QEAQLLSQPW QCQLYQGERL |
| 421 | LGAAVYFTEL SSPGAQRSGR APGALPAGHL LLFLILGVLS |
| | LLLLVTGAFG FHLWRRQWRP |
| 481 | RRFSALEQGI HPPQAQSKIE ELEQEPEPEP EPEPEPEPEP |
| | EPEQL |

In accordance with the presently disclosed subject matter, a “LAG-3 nucleic acid molecule” refers to a polynucleotide encoding a LAG-3 polypeptide.

Natural Killer Cell Receptor 2B4 (2B4) mediates non-MHC restricted cell killing on NK cells and subsets of T cells. The 2B4-S isoform is believed to be an activating receptor, and the 2B4-L isoform believed to be a negative immune regulator of immune cells. 2B4 becomes engaged upon binding its high-affinity ligand, CD48. 2B4 contains a tyrosine-based switch motif, a molecular switch that allows the protein to associate with various phosphatases. 2B4 has also been designated CD244 (cluster of differentiation 244).

In accordance with the presently disclosed subject matter, a 2B4 polypeptide can have an amino acid sequence that is at least about 85%, about 90%, about 95%, about 96%, about 97%, about 98%, about 99% or about 100% homologous to UniProtKB/Swiss-Prot Ref. No.: Q9BZW8.2 (SEQ ID NO: 23) or fragments thereof, and/or may optionally comprise up to one or up to two or up to three conservative amino acid substitutions.

SEQ ID NO: 23 is provided below:

[SEQ ID NO: 23]

| | |
|---|---|
| 1 | MLGQVVTLIL LLLLKVYQGK GCQGSADHVV SISGVPLQLQ |
| | PNSIQTKVDS IAWKKLLPSQ |
| 61 | NGFHHILKWE NGSLPSNTSN DRFSFIVKNL SLLIKAAQQQ |
| | DSGLYCLEVT SISGKVQTAT |
| 121 | FQVFVFESLL PDKVEKPRLQ GQGKILDRGR CQVALSCLVS |
| | RDGNVSYAWY RGSKLIQTAG |
| 181 | NLTYLDEEVD INGTHTYTCN VSNPVSWESH TLNLTQDCQN |
| | AHQEFRFWPF LVIIVILSAL |
| 241 | FLGTLACFCV WRRKRKEKQS ETSPKEFLTI YEDVKDLKTR |
| | RNHEQEQTFP GGGSTIYSMI |
| 301 | QSQSSAPTSQ EPAYTLYSLI QPSRKSGSRK RNHSPSFNST |
| | IYEVIGKSQP KAQNPARLSR |
| 361 | KELENFDVYS |

In accordance with the presently disclosed subject matter, a “2B4 nucleic acid molecule” refers to a polynucleotide encoding a 2B4 polypeptide.

B- and T-lymphocyte attenuator (BTLA) expression is induced during activation of T cells, and BTLA remains expressed on Th1 cells but not Th2 cells. Like PD1 and CTLA4, BTLA interacts with a B7 homolog, B7H4. However, unlike PD-1 and CTLA-4, BTLA displays T-Cell inhibition via interaction with tumor necrosis family receptors (TNF-R), not just the B7 family of cell surface receptors. BTLA is a ligand for tumor necrosis factor (receptor) superfamily, member 14 (TNFRSF14), also known as herpes virus entry mediator (HVEM). BTLA-HVEM complexes negatively regulate T-cell immune responses. BTLA activation has been shown to inhibit the function of cancer-specific human CD8⁺ T cells. BTLA has also been designated as CD272 (cluster of differentiation 272).

In accordance with the presently disclosed subject matter, a BTLA polypeptide can have an amino acid sequence that is at least about 85%, about 90%, about 95%, about 96%, about 97%, about 98%, about 99% or about 100% homologous to UniProtKB/Swiss-Prot Ref. No.: Q7Z6A9.3 (SEQ ID NO: 24) or fragments thereof, and/or may optionally comprise up to one or up to two or up to three conservative amino acid substitutions.

SEQ ID NO: 24 is provided below:

[SEQ ID NO: 24]

| | |
|---|---|
| 1 | MKTLPAMLGT GKLFWVFFLI PYLDIWNIHG KESCDVQLYI |
| | KRQSEHSILA GDPFELECPV |
| 61 | KYCANRPHVT WCKLNGTTCV KLEDRQTSWK EEKNISFFIL |
| | HFEPVLPNDN GSYRCSANFQ |
| 121 | SNLIESHSTT LYVTDVKSAS ERPSKDEMAS RPWLLYRLLP |
| | LGGLPLLITT CFCLFCCLRR |
| 181 | HQGKQNELSD TAGREINLVD AHLKSEQTEA STRQNSQVLL |
| | SETGIYDNDP DLCFRMQEGS |
| 241 | EVYSNPCLEE NKPGIVYASL NHSVIGPNSR LARNVKEAPT |
| | EYASICVRS |

In accordance with the presently disclosed subject matter, a “BTLA nucleic acid molecule” refers to a polynucleotide encoding a BTLA polypeptide.

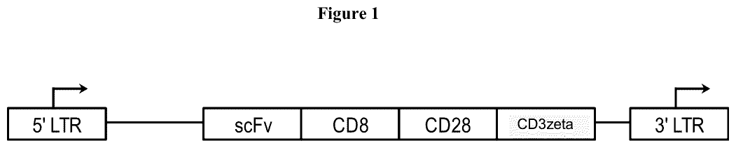

In certain embodiments, the CAR comprises, from 5′ to 3′, an extracellular antigen-binding region that comprises an scFv that specifically binds to a Trp1 polypeptide, a transmembrane domain comprising a CD8 polypeptide, and an intracellular domain comprising a co-stimulatory signaling region that comprises a CD28 polypeptide and a CD3zeta polypeptide, as shown in FIG. 1. As shown in FIG. 1, the CAR also comprises a signal peptide or a leader covalently joined to the 5′ terminus of the extracellular antigen-binding domain. In certain embodiments, the signal peptide comprises amino acids having the sequence set forth in SEQ ID NO: 12. In certain embodiments, the scFv comprises the sequences provided in Table 1.

In certain embodiments, the CAR of the presently disclosed subject matter can further comprise an inducible promoter, for expressing nucleic acid sequences in human cells. Promoters for use in expressing CAR genes can be a constitutive promoter, such as ubiquitin C (UbiC) promoter.

The presently disclosed subject matter also provides isolated nucleic acid molecule encoding the MDA-targeted CAR described herein or a functional portion thereof. In certain embodiments, the isolated nucleic acid molecule encodes a presently disclosed MDA-targeted CAR comprising an scFv that specifically binds to an MDA polypeptide (e.g., a Trp1 polypeptide), a transmembrane domain comprising a CD8 polypeptide, and an intracellular domain comprising a co-stimulatory signaling region comprising a CD28 polypeptide and a CD3zeta polypeptide.

In certain embodiments, the isolated nucleic acid molecule comprises the nucleotide sequence of SEQ ID NO: 25, which is provided below.

[SEQ ID NO: 25]
ATGGCCTTACCAGTGACCGCCTTGCTCCTGCCGCTGGCCTTGCTGCTCCA
CGCCGAGGTTCAGCTGCAGCAGTCTGGGGCTGAGCTTGTGAGGCCAGGGG
CCTTGGTCAAGTTGTCCTGCAAAACTTCTGGCTTCAACATTAAAGACTAC
TTTTTACACTGGGTGAGACAGAGGCCTGACCAGGGCCTGGAGTGGATTGG
ATGGATTAATCCTGATAATGGTAATACTGTTTATGACCCGAAGTTTCAGG
GCACGGCCAGTTTAACAGCAGACACATCCTCCAACACAGTCTACTTGCAG
CTCAGCGGCCTGACATCTGAGGACACTGCCGTCTATTTCTGTACTCGGAG
GGACTATACTTATGAAAAGGCTGCTCTGGACTACTGGGGTCAGGGAGCCT
CAGTCATCGTCTCCTCAGGTGGAGGTGGATCAGGTGGAGGTGGATCTGGT
GGAGGTGGATCTGCCTTCCAGATGTCTCAGTCTCCAGCCTCCCTATCTGC
ATCTGTGGGAGAAACTGTCACCATCACATGTCGAGCAAGTGGAAATATTT
ACAATTATTTAGCATGGTATCAGCAGAAACAGGGAAAATCTCCTCACCTC
CTGGTCTATGATGCAAAAACCTTAGCAGATGGTGTGCCATCAAGGTTCAG
TGGCAGTGGCTCAGGGACACAATATTCTCTCAAGATTAGCAGCCTGCAGA
CTGAAGATTCTGGGAATTATTACTGTCAACATTTTTGGAGTCTTCCATTC
ACGTTCGGCTCGGGGACAAAGTTGGAAATAAAAGCGGCCGCATCTACTAC
TACCAAGCCAGTGCTGCGAACTCCCTCACCTGTGCACCCTACCGGGACAT
CTCAGCCCCAGAGACCAGAAGATTGTCGGCCCCGTGGCTCAGTGAAGGGG
ACCGGATTGGACTTCGCCTGTGATATTTACATCTGGGCACCCTTGGCCGG
AATCTGCGTGGCCCTTCTGCTGTCCTTGATCATCACTCTCATCTGCTACA
ATAGTAGAAGGAACAGACTCCTTCAAAGTGACTACATGAACATGACTCCC
CGGAGGCCTGGGCTCACTCGAAAGCCTTACCAGCCCTACGCCCCTGCCAG
AGACTTTGCAGCGTACCGCCCCAGAGCAAAATTCAGCAGGAGTGCAGAGA
CTGCTGCCAACCTGCAGGACCCCAACCAGCTCTACAATGAGCTCAATCTA
GGGCGAAGAGAGGAATATGACGTCTTGGAGAAGAAGCGGGCTCGGGATCC
AGAGATGGGAGGCAAACAGCAGAGGAGGAGGAACCCCCAGGAAGGCGTAT
ACAATGCACTGCAGAAAGACAAGATGGCAGAAGCCTACAGTGAGATCGGC
ACAAAAGGCGAGAGGCGGAGAGGCAAGGGGCACGATGGCCTTTACCAGGG
TCTCAGCACTGCCACCAAGGACACCTATGATGCCCTGCATATGCAGACCC
TGGCCCCTCGCTAA

The isolated nucleic acid molecule having the nucleotide sequence of SEQ ID NO: 25 encodes a Trp1-targeted CAR comprising an scFv that comprises a heavy chain variable region comprising amino acids having the sequence set forth in SEQ ID NO: 7, a light chain variable region comprising amino acids having the sequence set forth in SEQ ID NO: 8, and a linker having an amino acid sequence of SEQ ID NO: 11 positioned between the heavy chain variable region and the light chain variable region, a transmembrane domain comprising a CD8 polypeptide comprising the amino acid sequence set forth in SEQ ID NO: 30, an intracellular domain comprising a co-stimulatory signaling region comprising a CD28 polypeptide comprising an amino acid sequence of amino acids 178 to 218 of SEQ ID NO: 35 and a CD3zeta polypeptide comprising the amino acid sequence set forth in SEQ ID NO: 34.

In certain embodiments, the isolated nucleic acid molecule encodes a functional portion of a presently disclosed MDA-targeted CAR. As used herein, the term “functional portion” refers to any portion, part or fragment of a presently disclosed MDA-targeted CAR, which portion, part or fragment retains the biological activity of the MDA-targeted CAR (the parent CAR). For example, functional portions encompass the portions, parts or fragments of a presently disclosed MDA-targeted CAR that retains the ability to recognize a target cell, to treat a disease, e.g., melanoma, to a similar, same, or even a higher extent as the parent CAR. In certain embodiments, an isolated nucleic acid molecule encoding a functional portion of a presently disclosed MDA-targeted CAR can encode a protein comprising, e.g., about 10%, about 20%, about 25%, about 30%, about 35%, about 40%, about 45%, about 50%, about 55%, about 60%, about 65%, about 70%, about 75%, about 80%, about 85%, about 90%, and about 95%, or more of the parent CAR.

### IV. Immunoresponsive Cells

The presently disclosed subject matter provides immunoresponsive cells comprising and/or expressing a CAR that comprises an extracellular antigen-binding domain, a transmembrane domain and an intracellular domain, wherein the extracellular antigen-binding domain specifically binds to an MDA polypeptide (e.g., a Trp1 polypeptide) as described above. The immunoresponsive cells can be transduced with a presently disclosed CAR such that the cells express the CAR. The presently disclosed subject matter also provides methods of using such cells for the treatment of a tumor, e.g., melanoma. The immunoresponsive cells of the presently disclosed subject matter can be cells of the lymphoid lineage. The lymphoid lineage, comprising B, T and natural killer (NK) cells, provides for the production of antibodies, regulation of the cellular immune system, detection of foreign agents in the blood, detection of cells foreign to the host, and the like. Non-limiting examples of immunoresponsive cells of the lymphoid lineage include T cells, Natural Killer (NK) cells, embryonic stem cells, and pluripotent stem cells (e.g., those from which lymphoid cells may be differentiated). T cells can be lymphocytes that mature in the thymus and are chiefly responsible for cell-mediated immunity. T cells are involved in the adaptive immune system. The T cells of the presently disclosed subject matter can be any type of T cells, including, but not limited to, helper T cells, cytotoxic T cells, memory T cells (including central memory T cells, stem-cell-like memory T cells (or stem-like memory T cells), and two types of effector memory T cells: e.g., TEM cells and TEMRA cells, Regulatory T cells (also known as suppressor T cells), Natural killer T cells, Mucosal associated invariant T cells, and γδ T cells. Cytotoxic T cells (CTL or killer T cells) are a subset of T lymphocytes capable of inducing the death of infected somatic or tumor cells. In certain embodiments, the CAR-expressing T cells express Foxp3 to achieve and maintain a T regulatory phenotype.

Natural killer (NK) cells can be lymphocytes that are part of cell-mediated immunity and act during the innate immune response. NK cells do not require prior activation in order to perform their cytotoxic effect on target cells.

The immunoresponsive cells of the presently disclosed subject matter can express an extracellular antigen-binding domain (e.g., an scFv, a Fab that is optionally crosslinked, or a F(ab)₂) that specifically binds to an MDA polypeptide (e.g., a Trp1 polypeptide), for the treatment of cancer, e.g., melanoma. Such immunoresponsive cells can be administered to a subject (e.g., a human subject) in need thereof for the treatment of cancer, e.g., melanoma. In certain embodiments, the immunoresponsive cells are T cells. The T cells can be CD4⁺ T cells, CD8⁺ T cells, or a combination/mixture of CD4⁺ T cells and CD8⁺ T cells. In certain embodiments, the T cells are CD4⁺ T cells. In certain embodiments, the T cells are CD8⁺ T cells. In certain embodiments, the T cells are a mixture of CD4⁺ T cells and CD8⁺ T cells.

A presently disclosed immunoresponsive cell can further include at least one recombinant or exogenous co-stimulatory ligand. For example, a presently disclosed immunoresponsive cell can be further transduced with at least one co-stimulatory ligand, such that the immunoresponsive cell co-expresses or is induced to co-express the MDA-targeted CAR and the at least one co-stimulatory ligand. The interaction between the MDA-targeted CAR and at least one co-stimulatory ligand provides a non-antigen-specific signal important for full activation of an immunoresponsive cell (e.g., T cell). Co-stimulatory ligands include, but are not limited to, members of the tumor necrosis factor (TNF) superfamily, and immunoglobulin (Ig) superfamily ligands. TNF is a cytokine involved in systemic inflammation and stimulates the acute phase reaction. Its primary role is in the regulation of immune cells. Members of TNF superfamily share a number of common features. The majority of TNF superfamily members are synthesized as type II transmembrane proteins (extracellular C-terminus) containing a short cytoplasmic segment and a relatively long extracellular region. TNF superfamily members include, without limitation, nerve growth factor (NGF), CD40L (CD40L)/CD154, CD137L/4-1BBL, TNF-α, CD134L/OX40L/CD252, CD27L/CD70, Fas ligand (FasL), CD30L/CD153, tumor necrosis factor beta (TNFβ)/lymphotoxin-alpha (LTα), lymphotoxin-beta (LTβ), CD257/B cell-activating factor (BAFF)/Blys/THANK/Tall-1, glucocorticoid-induced TNF Receptor ligand (GITRL), and TNF-related apoptosis-inducing ligand (TRAIL), LIGHT (TNFSF14). The immunoglobulin (Ig) superfamily is a large group of cell surface and soluble proteins that are involved in the recognition, binding, or adhesion processes of cells. These proteins share structural features with immunoglobulins—they possess an immunoglobulin domain. Immunoglobulin superfamily ligands include, but are not limited to, CD80 and CD86, both ligands for CD28, PD-L1/(B7-H1) that ligands for PD-1. In certain embodiments, the at least one co-stimulatory ligand is selected from the group consisting of 4-1BBL, CD80, CD86, CD70, OX40L, CD48, TNFRSF14, PD-L1, and combinations thereof. In certain embodiments, the immunoresponsive cell comprises one recombinant co-stimulatory ligand that is 4-1BBL. In certain embodiments, the immunoresponsive cell comprises two recombinant co-stimulatory ligands that are 4-1BBL and CD80. CARs comprising at least one co-stimulatory ligand are described in U.S. Pat. No. 8,389,282, which is incorporated by reference in its entirety.

Furthermore, a presently disclosed immunoresponsive cell can further comprise at least one exogenous cytokine. For example, a presently disclosed immunoresponsive cell can be further transduced with at least one cytokine, such that the immunoresponsive cell secretes the at least one cytokine as well as expresses the MDA-targeted CAR. In certain embodiments, the at least one cytokine is selected from the group consisting of IL-2, IL-3, IL-6, IL-7, IL-11, IL-12, IL-15, IL-17, and IL-21. In certain embodiments, the cytokine is IL-12.

The MDA-specific or MDA-targeted human lymphocytes that can be used in peripheral donor lymphocytes, e.g., those disclosed in Sadelain, M., et al. 2003 Nat Rev Cancer 3:35-45 (disclosing peripheral donor lymphocytes genetically modified to express CARs), in Morgan, R. A., et al. 2006 Science 314:126-129 (disclosing peripheral donor lymphocytes genetically modified to express a full-length tumor antigen-recognizing T cell receptor complex comprising the α and β heterodimer), in Panelli, M. C., et al. 2000 J Immunol 164:495-504; Panelli, M. C., et al. 2000 J Immunol 164:4382-4392 (disclosing lymphocyte cultures derived from tumor infiltrating lymphocytes (TILs) in tumor biopsies), and in Dupont, J., et al. 2005 Cancer Res 65:5417-5427; Papanicolaou, G. A., et al. 2003 Blood 102:2498-2505 (disclosing selectively in vitro-expanded antigen-specific peripheral blood leukocytes employing artificial antigen-presenting cells (AAPCs) or pulsed dendritic cells). The immunoresponsive cells (e.g., T cells) can be autologous, non-autologous (e.g., allogeneic), or derived in vitro from engineered progenitor or stem cells.

In certain embodiments, a presently disclosed immunoresponsive cell (e.g., T cell) expresses from about 1 to about 5, from about 1 to about 4, from about 2 to about 5, from about 2 to about 4, from about 3 to about 5, from about 3 to about 4, from about 4 to about 5, from about 1 to about 2, from about 2 to about 3, from about 3 to about 4, or from about 4 to about 5 vector copy numbers/cell of a presently disclosed MDA-targeted CAR.

In certain embodiments, a presently disclosed immunoresponsive cell (e.g., T cell) can be additionally modified to express antagonistic scFvs with immune regulatory functions (“armored CAR T cells”). For example, upon activation of a presently disclosed CAR to a cognate antigen (e.g., an MDA (e.g., Trp1)), armored CAR modified T cells can be induced to express scFvs antagonistic to an inhibitory T cell receptor (e.g., an inhibitory PD-1 T cell receptor, an inhibitory CTLA-4 T cell receptor, an inhibitory PD-L1 T cell receptor, or an inhibitory LAG3 T cell receptor) on both infused CAR modified T cells and endogenous anti-tumor T cells enhancing anti-tumor effector function. Details on armored CAR T cells are disclosed in WO2014134165, the contents of which are herein incorporated by reference.

### V. Vectors

Genetic modification of immunoresponsive cells (e.g., T cells, NK cells) can be accomplished by transducing a substantially homogeneous cell composition with a recombinant DNA or RNA construct. The vector can be a retroviral vector (e.g., gamma retroviral), which is employed for the introduction of the DNA or RNA construct into the host cell genome. For example, a polynucleotide encoding the MDA-targeted CAR can be cloned into a retroviral vector and expression can be driven from its endogenous promoter, from the retroviral long terminal repeat, or from an alternative internal promoter.

Non-viral vectors or RNA may be used as well. Random chromosomal integration, or targeted integration (e.g., using a nuclease, transcription activator-like effector nucleases (TALENs), Zinc-finger nucleases (ZFNs), and/or clustered regularly interspaced short palindromic repeats (CRISPRs), or transgene expression (e.g., using a natural or chemically modified RNA) can be used.

For initial genetic modification of the cells to provide MDA-targeted CAR expressing cells, a retroviral vector is generally employed for transduction, however any other suitable viral vector or non-viral delivery system can be used. For subsequent genetic modification of the cells to provide cells comprising an antigen presenting complex comprising at least two co-stimulatory ligands, retroviral gene transfer (transduction) likewise proves effective. Combinations of retroviral vector and an appropriate packaging line are also suitable, where the capsid proteins will be functional for infecting human cells. Various amphotropic virus-producing cell lines are known, including, but not limited to, PA12 (Miller, et al. (1985) Mol. Cell. Biol. 5:431-437); PA317 (Miller, et al. (1986) Mol. Cell. Biol. 6:2895-2902); and CRIP (Danos, et al. (1988) Proc. Natl. Acad. Sci. USA 85:6460-6464). Non-amphotropic particles are suitable too, e.g., particles pseudotyped with VSVG, RD114 or GALV envelope and any other known in the art.

Possible methods of transduction also include direct co-culture of the cells with producer cells, e.g., by the method of Bregni, et al. (1992) Blood 80:1418-1422, or culturing with viral supernatant alone or concentrated vector stocks with or without appropriate growth factors and polycations, e.g., by the method of Xu, et al. (1994) Exp. Hemat. 22:223-230; and Hughes, et al. (1992) J. Clin. Invest. 89:1817.

Transducing viral vectors can be used to express a co-stimulatory ligand and/or to secret a cytokine (e.g., 4-1BBL and/or IL-12) in an immunoresponsive cell. Preferably, the chosen vector exhibits high efficiency of infection and stable integration and expression (see, e.g., Cayouette et al., Human Gene Therapy 8:423-430, 1997; Kido et al., Current Eye Research 15:833-844, 1996; Bloomer et al., Journal of Virology 71:6641-6649, 1997; Naldini et al., Science 272:263 267, 1996; and Miyoshi et al., Proc. Natl. Acad. Sci. U.S.A. 94:10319, 1997). Other viral vectors that can be used include, for example, adenoviral, lentiviral, and adeno-associated viral (“AAV”) vectors, vaccinia virus, a bovine papilloma virus, or a herpes virus, such as Epstein-Barr Virus (also see, for example, the vectors of Miller, Human Gene Therapy 15-14, 1990; Friedman, Science 244:1275-1281, 1989; Eglitis et al., BioTechniques 6:608-614, 1988; Tolstoshev et al., Current Opinion in Biotechnology 1:55-61, 1990; Sharp, The Lancet 337:1277-1278, 1991; Cornetta et al., Nucleic Acid Research and Molecular Biology 36:311-322, 1987; Anderson, Science 226:401-409, 1984; Moen, Blood Cells 17:407-416, 1991; Miller et al., Biotechnology 7:980-990, 1989; Le Gal La Salle et al., Science 259:988-990, 1993; and Johnson, Chest 107:77S-83S, 1995). Retroviral vectors are particularly well developed and have been used in clinical settings (Rosenberg et al., N. Engl. J. Med 323:370, 1990; Anderson et al., U.S. Pat. No. 5,399,346). In certain embodiments, the vector is a lentiviral (“LV”) vector.

In certain non-limiting embodiments, the vector expressing a presently disclosed MDA-targeted CAR is a retroviral vector, e.g., an oncoretroviral vector.

Non-viral approaches can also be employed for the expression of a protein in cell. For example, a nucleic acid molecule can be introduced into a cell by administering the nucleic acid in the presence of lipofection (Feigner et al., Proc. Nat'l. Acad. Sci. U.S.A. 84:7413, 1987; Ono et al., Neuroscience Letters 17:259, 1990; Brigham et al., Am. J. Med. Sci. 298:278, 1989; Staubinger et al., Methods in Enzymology 101:512, 1983), asialoorosomucoid-polylysine conjugation (Wu et al., Journal of Biological Chemistry 263:14621, 1988; Wu et al., Journal of Biological Chemistry 264:16985, 1989), or by micro-injection under surgical conditions (Wolff et al., Science 247:1465, 1990). Other non-viral means for gene transfer include transfection in vitro using calcium phosphate, DEAE dextran, electroporation, and protoplast fusion. Liposomes can also be potentially beneficial for delivery of DNA into a cell. Transformation of normal genes into the affected tissues of a subject can also be accomplished by transferring a normal nucleic acid into a cultivatable cell type ex vivo (e.g., an autologous or heterologous primary cell or progeny thereof), after which the cell (or its descendants) are injected into a targeted tissue or are injected systemically. Recombinant receptors can also be derived or obtained using transposases or targeted nucleases (e.g., Zinc finger nucleases, meganucleases, or TALE nucleases). Transient expression may be obtained by RNA electroporation.

cDNA expression for use in polynucleotide therapy methods can be directed from any suitable promoter (e.g., the human cytomegalovirus (CMV), simian virus 40 (SV40), or metallothionein promoters), and regulated by any appropriate mammalian regulatory element or intron (e.g., the elongation factor 1α enhancer/promoter/intron structure). For example, if desired, enhancers known to preferentially direct gene expression in specific cell types can be used to direct the expression of a nucleic acid. The enhancers used can include, without limitation, those that are characterized as tissue- or cell-specific enhancers. Alternatively, if a genomic clone is used as a therapeutic construct, regulation can be mediated by the cognate regulatory sequences or, if desired, by regulatory sequences derived from a heterologous source, including any of the promoters or regulatory elements described above.

The resulting cells can be grown under conditions similar to those for unmodified cells, whereby the modified cells can be expanded and used for a variety of purposes.

### VI. Polypeptides and Analogs and Polynucleotides

Also included in the presently disclosed subject matter are extracellular antigen-binding domains that specifically binds to an MDA (e.g., Trp1) (e.g., an scFv, a Fab, or a (Fab)₂), CD3zeta, CD8, CD28, etc. polypeptides or fragments thereof, and polynucleotides encoding thereof that are modified in ways that enhance their anti-tumor activity when expressed in an immunoresponsive cell. The presently disclosed subject matter provides methods for optimizing an amino acid sequence or a nucleic acid sequence by producing an alteration in the sequence. Such alterations may comprise certain mutations, deletions, insertions, or post-translational modifications. The presently disclosed subject matter further comprises analogs of any naturally-occurring polypeptide of the presently disclosed subject matter. Analogs can differ from a naturally-occurring polypeptide of the presently disclosed subject matter by amino acid sequence differences, by post-translational modifications, or by both. Analogs of the presently disclosed subject matter can generally exhibit at least about 85%, about 90%, about 91%, about 92%, about 93%, about 94%, about 95%, about 96%, about 97%, about 98%, about 99% or more identity or homology with all or part of a naturally-occurring amino, acid sequence of the presently disclosed subject matter. The length of sequence comparison is at least about 5, about 10, about 15, about 20, about 25, about 50, about 75, about 100 or more amino acid residues. Again, in an exemplary approach to determining the degree of identity, a BLAST program may be used, with a probability score between e⁻³ and e⁻¹⁰⁰ indicating a closely related sequence. Modifications comprise in vivo and in vitro chemical derivatization of polypeptides, e.g., acetylation, carboxylation, phosphorylation, or glycosylation; such modifications may occur during polypeptide synthesis or processing or following treatment with isolated modifying enzymes. Analogs can also differ from the naturally-occurring polypeptides of the presently disclosed subject matter by alterations in primary sequence. These include genetic variants, both natural and induced (for example, resulting from random mutagenesis by irradiation or exposure to ethanemethylsulfate or by site-specific mutagenesis as described in Sambrook, Fritsch and Maniatis, Molecular Cloning: A Laboratory Manual (2d ed.), CSH Press, 1989, or Ausubel et al., supra). Also included are cyclized peptides, molecules, and analogs which contain residues other than L-amina acids, e.g., D-amino acids or non-naturally occurring or synthetic amino acids, e.g., beta (β) or gamma (γ) amino acids.

In addition to full-length polypeptides, the presently disclosed subject matter also provides fragments of any one of the polypeptides or peptide domains of the presently disclosed subject matter. A fragment can be at least about 5, about 10, about 13, or about 15 amino acids. In some embodiments, a fragment is at least about 20 contiguous amino acids, at least about 30 contiguous amino acids, or at least about 50 contiguous amino acids. In some embodiments, a fragment is at least about 60 to about 80, about 100, about 200, about 300 or more contiguous amino acids. Fragments of the presently disclosed subject matter can be generated by methods known to those of ordinary skill in the art or may result from normal protein processing (e.g., removal of amino acids from the nascent polypeptide that are not required for biological activity or removal of amino acids by alternative mRNA splicing or alternative protein processing events).

Non-protein analogs have a chemical structure designed to mimic the functional activity of a protein of the presently disclosed subject matter. Such analogs are administered according to methods of the presently disclosed subject matter. Such analogs may exceed the physiological activity of the original polypeptide. Methods of analog design are well known in the art, and synthesis of analogs can be carried out according to such methods by modifying the chemical structures such that the resultant analogs increase the anti-neoplastic activity of the original polypeptide when expressed in an immunoresponsive cell. The protein analogs can be relatively resistant to in vivo degradation, resulting in a more prolonged therapeutic effect upon administration. Assays for measuring functional activity include, but are not limited to, those described in the Examples below.

In accordance with the presently disclosed subject matter, the polynucleotides encoding an extracellular antigen-binding domain that specifically binds to an MDA (e.g., Trp1) (e.g., an scFv, a Fab, or a (Fab)₂), CD3zeta, CD8, CD28) can be modified by codon optimization. Codon optimization can alter both naturally occurring and recombinant gene sequences to achieve the highest possible levels of productivity in any given expression system. Factors that are involved in different stages of protein expression include codon adaptability, mRNA structure, and various cis-elements in transcription and translation. Any suitable codon optimization methods or technologies that are known to ones skilled in the art can be used to modify the polynucleotides of the presently disclosed subject matter, including, but not limited to, OptimumGene™, Encor optimization, and Blue Heron.

### VIII. Administration

MDA-targeted CARs and immunoresponsive cells comprising thereof of the presently disclosed subject matter can be provided systemically or directly to a subject for treating or preventing a neoplasia. In certain embodiments, MDA-targeted CARs, and immunoresponsive cells comprising thereof are directly injected into an organ of interest (e.g., an organ affected by a neoplasia). Alternatively or additionally, the MDA-targeted CARs and immunoresponsive cells comprising thereof are provided indirectly to the organ of interest, for example, by administration into the circulatory system (e.g., the tumor vasculature). Expansion and differentiation agents can be provided prior to, during or after administration of cells and compositions to increase production of T cells in vitro or in vivo.

MDA-targeted CARs and immunoresponsive cells comprising thereof of the presently disclosed subject matter can be administered in any physiologically acceptable vehicle, normally intravascularly, although they may also be introduced into bone or other convenient site where the cells may find an appropriate site for regeneration and differentiation (e.g., thymus). In certain embodiments, at least about 1×10⁵ cells can be administered, eventually reaching about 1×10¹⁰ or more. In certain embodiments, at least about 1×10⁶ cells can be administered. A cell population comprising immunoresponsive cells comprising an MDA-targeted CAR can comprise a purified population of cells. Those skilled in the art can readily determine the percentage of immunoresponsive cells in a cell population using various well-known methods, such as fluorescence activated cell sorting (FACS). The ranges of purity in cell populations comprising genetically modified immunoresponsive cells expressing an MDA-specific CAR can be from about 50% to about 55%, from about 55% to about 60%, from about 65% to about 70%, from about 70% to about 75%, from about 75% to about 80%, from about 80% to about 85%; from about 85% to about 90%, from about 90% to about 95%, or from about 95 to about 100%. Dosages can be readily adjusted by those skilled in the art (e.g., a decrease in purity may require an increase in dosage). The immunoresponsive cells can be introduced by injection, catheter, or the like. If desired, factors can also be included, including, but not limited to, interleukins, e.g. IL-2, IL-3, IL 6, IL-11, IL-7, IL-12, IL-15, IL-21, as well as the other interleukins, the colony stimulating factors, such as G-, M- and GM-CSF, interferons, e.g., γ-interferon.

In certain embodiments, compositions of the presently disclosed subject matter comprise pharmaceutical compositions comprising immunoresponsive cells comprising an MDA-targeted CAR and a pharmaceutically acceptable carrier. Administration can be autologous or non-autologous. For example, immunoresponsive cells expressing an MDA-targeted CAR and compositions comprising thereof can be obtained from one subject, and administered to the same subject or a different, compatible subject. Peripheral blood derived T cells of the presently disclosed subject matter or their progeny (e.g., in vivo, ex vivo or in vitro derived) can be administered via localized injection, including catheter administration, systemic injection, localized injection, intravenous injection, or parenteral administration. When administering a pharmaceutical composition of the presently disclosed subject matter (e.g., a pharmaceutical composition comprising immunoresponsive cells comprising an MDA-targeted CAR), it can be formulated in a unit dosage injectable form (solution, suspension, emulsion).

In certain embodiments, compositions of the presently disclosed subject matter can comprise one or more MDA-targeted CAR disclosed herein, and a pharmaceutically acceptable carrier.

### IX. Formulations

Immunoresponsive cells comprising a presently disclosed an MDA-targeted CAR and compositions comprising thereof can be conveniently provided as sterile liquid preparations, e.g., isotonic aqueous solutions, suspensions, emulsions, dispersions, or viscous compositions, which may be buffered to a selected pH. Liquid preparations are normally easier to prepare than gels, other viscous compositions, and solid compositions. Additionally, liquid compositions are somewhat more convenient to administer, especially by injection. Viscous compositions, on the other hand, can be formulated within the appropriate viscosity range to provide longer contact periods with specific tissues. Liquid or viscous compositions can comprise carriers, which can be a solvent or dispersing medium containing, for example, water, saline, phosphate buffered saline, polyol (for example, glycerol, propylene glycol, liquid polyethylene glycol, and the like) and suitable mixtures thereof.

Sterile injectable solutions can be prepared by incorporating the compositions of the presently disclosed subject matter, e.g., a composition comprising immunoresponsive cells comprising a presently disclosed an MDA-targeted CAR, in the required amount of the appropriate solvent with various amounts of the other ingredients, as desired. Such compositions may be in admixture with a suitable carrier, diluent, or excipient such as sterile water, physiological saline, glucose, dextrose, or the like. The compositions can also be lyophilized. The compositions can contain auxiliary substances such as wetting, dispersing, or emulsifying agents (e.g., methylcellulose), pH buffering agents, gelling or viscosity enhancing additives, preservatives, flavoring agents, colors, and the like, depending upon the route of administration and the preparation desired. Standard texts, such as “REMINGTON'S PHARMACEUTICAL SCIENCE”, 17th edition, 1985, incorporated herein by reference, may be consulted to prepare suitable preparations, without undue experimentation.

Various additives which enhance the stability and sterility of the compositions, including antimicrobial preservatives, antioxidants, chelating agents, and buffers, can be added. Prevention of the action of microorganisms can be ensured by various antibacterial and antifungal agents, for example, parabens, chlorobutanol, phenol, sorbic acid, and the like. Prolonged absorption of the injectable pharmaceutical form can be brought about by the use of agents delaying absorption, for example, aluminium monostearate and gelatin. According to the presently disclosed subject matter, however, any vehicle, diluent, or additive used would have to be compatible with the immunoresponsive cells comprising an MDA-targeted CAR of the presently disclosed subject matter.

The compositions can be isotonic, i.e., they can have the same osmotic pressure as blood and lacrimal fluid. The desired isotonicity of the compositions of the presently disclosed subject matter may be accomplished using sodium chloride, or other pharmaceutically acceptable agents such as dextrose, boric acid, sodium tartrate, propylene glycol or other inorganic or organic solutes.

Viscosity of the compositions, if desired, can be maintained at the selected level using a pharmaceutically acceptable thickening agent. Methylcellulose can be used because it is readily and economically available and is easy to work with. Other suitable thickening agents include, for example, xanthan gum, carboxymethyl cellulose, hydroxypropyl cellulose, carbomer, and the like. The concentration of the thickener can depend upon the agent selected. The important point is to use an amount that will achieve the selected viscosity. Obviously, the choice of suitable carriers and other additives will depend on the exact route of administration and the nature of the particular dosage form, e.g., liquid dosage form (e.g., whether the composition is to be formulated into a solution, a suspension, gel or another liquid form, such as a time release form or liquid-filled form).

Those skilled in the art will recognize that the components of the compositions should be selected to be chemically inert and will not affect the viability or efficacy of the immunoresponsive cells as described herein. This will present no problem to those skilled in the chemical and pharmaceutical arts, or problems can be readily avoided by reference to standard texts or by simple experiments (not involving undue experimentation), from this disclosure and the documents cited herein.

One consideration concerning the therapeutic use of the immunoresponsive cells disclosed herein is the quantity of cells necessary to achieve an optimal effect. The quantity of cells to be administered will vary for the subject being treated. In certain embodiments, from about 10⁴ to about 10¹⁰, from about 10⁵ to about 10⁹, or from about 10⁶ to about 10⁸ immunoresponsive cells of the presently disclosed subject matter are administered to a subject. More effective cells may be administered in even smaller numbers. In some embodiments, at least about 1×10⁸, at least about 2×10⁸, at least about 3×10⁸, at least about 4×10⁸, or at least about 5×10⁸ immunoresponsive cells of the presently disclosed subject matter are administered to a human subject. The precise determination of what would be considered an effective dose may be based on factors individual to each subject, including their size, age, sex, weight, and condition of the particular subject. Dosages can be readily ascertained by those skilled in the art from this disclosure and the knowledge in the art.

The skilled artisan can readily determine the amount of cells and optional additives, vehicles, and/or carrier in compositions and to be administered in methods disclosed herein. Typically, any additives (in addition to the active cell(s) and/or agent(s)) are present in an amount of from about 0.001% to about 50% by weight) solution in phosphate buffered saline, and the active ingredient is present in the order of micrograms to milligrams, such as from about 0.0001 wt % to about 5 wt %, from about 0.0001 wt % to about 1 wt %, from about 0.0001 wt % to about 0.05 wt %, from about 0.001 wt % to about 20 wt %, from about 0.01 wt % to about 10 wt %, or from about 0.05 wt % to about 5 wt %. For any composition to be administered to an animal or human, and for any particular method of administration, toxicity should be determined, such as by determining the lethal dose (LD) and LD50 in a suitable animal model e.g., a rodent such as a mouse; and, the dosage of the composition(s), concentration of components therein and timing of administering the composition(s), which elicit a suitable response. Such determinations do not require undue experimentation from the knowledge of the skilled artisan, this disclosure and the documents cited herein. And, the time for sequential administrations can be ascertained without undue experimentation.

### X. Methods of Treatment

Tumor Microenvironment. Tumors have a microenvironment that is hostile to the host immune response involving a series of mechanisms by malignant cells to protect themselves from immune recognition and elimination. This “hostile tumor microenvironment” comprises a variety of immune suppressive factors including infiltrating regulatory CD4⁺ T cells (Tregs), myeloid derived suppressor cells (MDSCs), tumor associated macrophages (TAMs), immune suppressive cytokines including IL-10 and TGF-β, and expression of ligands targeted to immune suppressive receptors expressed by activated T cells (CTLA-4 and PD-1). These mechanisms of immune suppression play a role in the maintenance of tolerance and suppressing inappropriate immune responses, however within the tumor microenvironment these mechanisms prevent an effective anti-tumor immune response. Collectively these immune suppressive factors can induce either marked anergy or apoptosis of adoptively transferred CAR modified T cells upon encounter with targeted tumor cells.

Challenges in tumor immunology. Effective tumor immunity requires recognition of tumor antigens and unopposed tumor elimination by immune effector cells. Tumor antigens must contain peptide epitopes that are presented by the tumor and can be recognized by specific cytotoxic T lymphocytes (CTLs). The primed CTLs must expand to a sufficient number and migrate to tumor sites, wherein they mature into effectors to perform their functions, which are enhanced by helper T cells and dampened by Tregs and inhibitory macrophages.

Targeted T cell therapy with engineered T lymphocytes. T cell engineering is a groundbreaking strategy to potentially resolve many previously observed shortcomings of earlier immunotherapeutic approaches. Researchers have reported dramatic complete remissions in relapsed (Brentjens, R. J., et al. Safety and persistence of adoptively transferred autologous CD19-targeted T cells in patients with relapsed or chemotherapy refractory B-cell leukemias. Blood 118, 4817-4828 (2011); Brentjens, R. J., et al. CD19-targeted T cells rapidly induce molecular remissions in adults with chemotherapy-refractory acute lymphoblastic leukemia. Science translational medicine 5, 177ra138 (2013)), chemorefractory leukemia and metastatic melanoma (Hunder, N. N., et al. Treatment of metastatic melanoma with autologous CD4+ T cells against NY-ESO-1. N. Engl. J. Med. 358, 2698-2703 (2008); Rosenberg, S. A., Restifo, N. P., Yang, J. C., Morgan, R. A. & Dudley, M. E. Adoptive cell transfer: a clinical path to effective cancer immunotherapy. Nat. Rev. Cancer 8, 299-308 (2008); Dudley, M. E., et al. Adoptive cell therapy for patients with metastatic melanoma: evaluation of intensive myeloablative chemoradiation preparative regimens. J Clin Oncol 26, 5233-5239 (2008)), obtained with autologous peripheral blood T cells targeted to a defined antigen (CD19 and NY-ESO-1, respectively).

Rationale for a genetic approach: Cell engineering can be used to redirect T cells toward tumor antigens and to enhance T cell function. One impetus for genetic T cell modification is the potential to enhance T cell survival and expansion and to offset T cell death, anergy, and immune suppression. The genetic targeting of T cells can also be refined to prevent undesired destruction of normal tissues.

Chimeric antigen receptors (CARs): Tumor-specific T cells can be generated by the transfer of genes that encode CARs (Brentjens, R. J., et al. Genetically targeted T cells eradicate systemic acute lymphoblastic leukemia xenografts. Clin.Cancer Res. 13, 5426-5435 (2007); Gade, T. P., et al. Targeted elimination of prostate cancer by genetically directed human T lymphocytes. Cancer Res. 65, 9080-9088 (2005); Maher, J., Brentjens, R. J., Gunset, G., Riviere, I. & Sadelain, M. Human T-lymphocyte cytotoxicity and proliferation directed by a single chimeric TCRzeta/CD28 receptor. Nat. Biotechnol. 20, 70-75 (2002); Kershaw, M. H., et al. Gene-engineered T cells as a superior adjuvant therapy for metastatic cancer. J Immunol 173, 2143-2150 (2004); Sadelain, M., Brentjens, R. & Riviere, I. The promise and potential pitfalls of chimeric antigen receptors. Curr Opin Immunol (2009); Hollyman, D., et al. Manufacturing validation of biologically functional T cells targeted to CD19 antigen for autologous adoptive cell therapy. J Immunother 32, 169-180 (2009)). Second-generation CARs comprise a tumor antigen-binding domain fused to an intracellular signaling domain capable of activating T cells and a co-stimulatory domain designed to augment T cell potency and persistence (Sadelain, M., Brentjens, R. & Riviere, I. The basic principles of chimeric antigen receptor design. Cancer discovery 3, 388-398 (2013)). CAR design can therefore reconcile antigen recognition with signal transduction, two functions that are physiologically borne by two separate complexes, the TCR heterodimer and the CD3 complex. The CAR's extracellular antigen-binding domain is usually derived from a murine monoclonal antibody (mAb) or from receptors or their ligands. Antigen recognition is therefore not MHC-restricted (Riviere, I., Sadelain, M. & Brentjens, R. J. Novel strategies for cancer therapy: the potential of genetically modified T lymphocytes. Curr Hematol Rep 3, 290-297 (2004); Stephan, M. T., et al. T cell-encoded CD80 and 4-1BBL induce auto- and transco-stimulation, resulting in potent tumor rejection. Nat. Med. 13, 1440-1449 (2007)) and is therefore applicable to any patient expressing the target antigen, using the same CAR. Antigen binding by the CARs triggers phosphorylation of immunoreceptor tyrosine-based activation motifs (ITAMs) in the intracellular domain, initiating a signaling cascade required for cytolysis induction, cytokine secretion, and proliferation. Because MHC restriction of antigen recognition is bypassed, the function of CAR-targeted T cells is not affected by HLA downregulation or defects in the antigen-processing machinery.

T cell requirements for expansion and survival: Proliferation of tumor-specific T cells is needed ex vivo and is desirable in vivo. T cell proliferation must be accompanied by T cell survival to permit absolute T cell expansion and persistence. To proliferate in response to antigen, T cells must receive two signals. One is provided by TCR recognition of antigenic peptide/MHC complexes displayed on the surface of antigen-presenting cells (APCs) (Sadelain (2009). The other is provided by a T cell co-stimulatory receptor, such as the CD28 or 4-1BB receptors. Whereas the cytolytic activity of T cells does not require concomitant co-stimulation, there is a critical need for the provision of co-stimulatory signals to sustain the antitumor functions of adoptively transferred T cells, as previously demonstrated (Maher (2002); Sadelain (2013); Krause, A., et al. Antigen-dependent CD28 signaling selectively enhances survival and proliferation in genetically modified activated human primary T lymphocytes. J Exp Med 188, 619-626 (1998); Gong, M. C., et al. Cancer patient T cells genetically targeted to prostate-specific membrane antigen specifically lyse prostate cancer cells and release cytokines in response to prostate-specific membrane antigen. Neoplasia. 1, 123-127 (1999); Lyddane, C., et al. Cutting Edge: CD28 controls dominant regulatory T cell activity during active immunization. J. Immunol. 176, 3306-3310 (2006).

Immune monitoring: Lymphocytes are multifunctional “drugs” that exhibit dynamically evolving effects after infusion. Upon antigen encounter, tumor-specific T cells activate and/or release a variety of proteins that can trigger tumor killing, T cell proliferation, and recruitment or immunomodulation of other immune cells. Thus, measuring which proteins are secreted from which cells, in what quantity, and at what time point yields profound insights into why a particular patient is or is not responding and provides critical feedback for designing more-effective trials.

For treatment, the amount administered is an amount effective in producing the desired effect. An effective amount can be provided in one or a series of administrations. An effective amount can be provided in a bolus or by continuous perfusion.

For adoptive immunotherapy using antigen-specific T cells, cell doses in the range of about 10⁶ to about 10¹⁰ (e.g., about 10⁹ or about 10⁶) are typically infused. Upon administration of the immunoresponsive cells into the subject and subsequent proliferation and growth, the immunoresponsive cells are induced that are specifically directed against one specific antigen (e.g., MDA). “Induction” of T cells can include inactivation of antigen-specific T cells such as by deletion or anergy. Inactivation is particularly useful to establish or reestablish tolerance such as in autoimmune disorders. The immunoresponsive cells of the presently disclosed subject matter can be administered by any methods known in the art, including, but not limited to, pleural administration, intravenous administration, subcutaneous administration, intranodal administration, intratumoral administration, intrathecal administration, intrapleural administration, intraperitoneal administration, and direct administration to the thymus. In one embodiment, the immunoresponsive cells and the compositions comprising thereof are intravenously administered to the subject in need.

The presently disclosed subject matter provides various methods of using the immunoresponsive cells (e.g., T cells) comprising an MDA-targeted CAR. For example, the presently disclosed subject matter provides methods of reducing tumor burden in a subject. In one non-limiting example, the method of reducing tumor burden comprises administering to the subject an effective amount of the presently disclosed immunoresponsive cells or a pharmaceutical composition comprising thereof, thereby inducing tumor cell death in the subject. The presently disclosed immunoresponsive cells or pharmaceutical composition comprising thereof can reduce the number of tumor cells, reduce tumor size, and/or eradicate the tumor in the subject. Non-limiting examples of suitable tumors include melanoma, glioblastoma multiforme, anaplastic astrocytoma, ependymoma, meningioma, and oligodendroglioma. In certain embodiments, the tumor is a melanoma.

In certain embodiments, the method further comprising pre-conditioning the subject prior to administering the immunoresponsive cells or pharmaceutical composition. Any suitable pre-conditioning treatments for immunotherapy can be applied. Non-limiting examples of pre-conditioning treatments include chemotherapy, radiotherapy, lymphodepleting treatment, total body irradiation, and a combination thereof. In certain embodiments, the pre-conditioning treatment is myeloablative. In certain embodiments, the pre-conditioning treatment is non-myeloablative. In certain embodiments, the pre-conditioning treatment is lymphodepleting. In certain embodiments, the pre-conditioning treatment can facilitate T cell expansion.

In certain embodiments, the pre-conditioning treatment is chemotherapy. In certain embodiments, the method further comprises administering to the subject a chemotherapeutic agent. In certain embodiments, the subject receives the chemotherapeutic agent prior to the immunoresponsive cells or pharmaceutical composition comprising thereof. Non-limiting examples of chemotherapeutic agents include docetaxel, cyclophosphamide, capecitabine, doxorubic, fludarabin, and a combination thereof. In certain embodiments, the chemotherapeutic agent is cyclophosphamide.

In certain embodiments, the pre-conditioning treatment is performed about 1 day, about 2 days, about 3 days, about 4 days, about 5 days, about 6 days, about 7 days, about 8 days, about 9 days, about 10 days, about 11 days, about 12 days, about 13 days, about 14 days, about 15 days, about 16 days, about 17 days, about 18 days, about 19 days, about 20 days, about 25 days, about 30 days, about 40 days, about 50 days or more, or any intermediate time period thereof, prior to the administration of the immunoresponsive cells or pharmaceutical composition comprising thereof.

In certain embodiments, the pre-conditioning treatment is performed between about 1 day to about 2 weeks. between about 1 week to about 2 weeks, between about 2 weeks to about 3 weeks, between about 3 weeks to about 4 weeks, or between about 4 weeks to about 5 weeks, prior to the administration of the immunoresponsive cells or pharmaceutical composition comprising thereof.

The presently disclosed subject matter also provides methods of increasing or lengthening survival of a subject having a neoplasia. In one non-limiting example, the method of increasing or lengthening survival of a subject having neoplasia comprises administering to the subject an effective amount of the presently disclosed immunoresponsive cells or a pharmaceutical composition comprising thereof, thereby increasing or lengthening survival of the subject. The method can reduce or eradicate tumor burden in the subject. The presently disclosed subject matter further provides methods for treating or preventing a neoplasia in a subject, comprising administering to the subject the presently disclosed immunoresponsive cells or a pharmaceutical composition comprising thereof.

Cancers whose growth may be inhibited using the immunoresponsive cells of the presently disclosed subject matter comprise cancers typically responsive to immunotherapy. Non-limiting examples of cancers for treatment include melanoma, glioblastoma multiforme, anaplastic astrocytoma, ependymoma, meningioma, and oligodendroglioma. In certain embodiments, the cancer is melanoma.

Suitable human subjects for therapy typically comprise two treatment groups that can be distinguished by clinical criteria. Subjects with “advanced disease” or “high tumor burden” are those who bear a clinically measurable tumor (e.g., melanoma). A clinically measurable tumor is one that can be detected on the basis of tumor mass (e.g., by palpation, CAT scan, sonogram, mammogram or X-ray; positive biochemical or histopathologic markers on their own are insufficient to identify this population). A pharmaceutical composition embodied in the presently disclosed subject matter is administered to these subjects to elicit an anti-tumor response, with the objective of palliating their condition. Ideally, reduction in tumor mass occurs as a result, but any clinical improvement constitutes a benefit. Clinical improvement comprises decreased risk or rate of progression or reduction in pathological consequences of the tumor (e.g., melanoma).

A second group of suitable subjects is known in the art as the “adjuvant group.” These are individuals who have had a history of neoplasia (e.g., melanoma), but have been responsive to another mode of therapy. The prior therapy can have included, but is not restricted to, surgical resection, radiotherapy, and traditional chemotherapy. As a result, these individuals have no clinically measurable tumor. However, they are suspected of being at risk for progression of the disease, either near the original tumor site, or by metastases. This group can be further subdivided into high-risk and low-risk individuals. The subdivision is made on the basis of features observed before or after the initial treatment. These features are known in the clinical arts, and are suitably defined for each different neoplasia. Features typical of high-risk subgroups are those in which the tumor (e.g., melanoma) has invaded neighboring tissues, or who show involvement of lymph nodes. Another group has a genetic predisposition to neoplasia (e.g., melanoma) but has not yet evidenced clinical signs of neoplasia (e.g., melanoma). For instance, women testing positive for a genetic mutation associated with breast cancer, but still of childbearing age, may wish to receive one or more of the MDA-specific CARs described herein in treatment prophylactically to prevent the occurrence of neoplasia until it is suitable to perform preventive surgery.

The subjects can have an advanced form of disease (e.g., melanoma), in which case the treatment objective can include mitigation or reversal of disease progression, and/or amelioration of side effects. The subjects can have a history of the condition, for which they have already been treated, in which case the therapeutic objective will typically include a decrease or delay in the risk of recurrence. Further modification can be introduced to the MDA-targeted CAR-expressing immunoresponsive cells (e.g., T cells) to avert or minimize the risks of immunological complications (known as “malignant T-cell transformation”), e.g., graft versus-host disease (GvHD), or when healthy tissues express the same target antigens as the tumor cells, leading to outcomes similar to GvHD. A potential solution to this problem is engineering a suicide gene into the MDA-targeted CAR-expressing T cells. Suitable suicide genes include, but are not limited to, Herpes simplex virus thymidine kinase (hsv-tk), inducible Caspase 9 Suicide gene (iCasp-9), and a truncated human epidermal growth factor receptor (EGFRt) polypeptide. In certain embodiments, the suicide gene is an EGFRt polypeptide. The EGFRt polypeptide can enable T cell elimination by administering anti-EGFR monoclonal antibody (e.g., cetuximab). EGFRt can be covalently joined to the 3′ terminus of the intracellular domain of the MDA-targeted CAR. The suicide gene can be included within the vector comprising nucleic acids encoding the presently disclosed MDA-targeted CARs. In this way, administration of a prodrug designed to activate the suicide gene (e.g., a prodrug (e.g., AP1903 that activates iCasp-9) during malignant T-cell transformation (e.g., GvHD) triggers apoptosis in the suicide gene-activated CAR-expressing T cells. The incorporation of a suicide gene into the a presently disclosed MDA-targeted CAR gives an added level of safety with the ability to eliminate the majority of CAR T cells within a very short time period. A presently disclosed immunoresponsive cell (e.g., a T cell) incorporated with a suicide gene can be pre-emptively eliminated at a given timepoint post CAR T cell infusion, or eradicated at the earliest signs of toxicity.

Immunomodulatory Agents: In accordance with the presently disclosed subject matter, the above-described various methods can further comprise administering to the subject at least one checkpoint immune blockade agent. Non-limiting examples of checkpoint immune blockade agents include an anti-4-1BB antibody, an anti-OX40 antibody, an anti-GITR antibody, an anti-PD-L1 antibody, an anti-CTLA-4 antibody, an anti-PD-1 antibody, an anti-LAG3 antibody, an anti-TNSF25 antibody, an anti-TIGT antibody, an anti-CD40 antibody, and combinations thereof.

### XI. Kits

The presently disclosed subject matter provides kits for the treatment or prevention of a neoplasia (e.g., melanoma). In certain embodiments, the kit comprises a therapeutic or prophylactic composition comprising an effective amount of presently disclosed immunoresponsive cells or a pharmaceutical composition comprising thereof in unit dosage form. In certain embodiments, the cells further express at least one co-stimulatory ligand. In some embodiments, the kit comprises a sterile container which contains a therapeutic or prophylactic vaccine; such containers can be boxes, ampules, bottles, vials, tubes, bags, pouches, blister-packs, or other suitable container forms known in the art. Such containers can be made of plastic, glass, laminated paper, metal foil, or other materials suitable for holding medicaments.

If desired, the immunoresponsive cells can be provided together with instructions for administering the cells to a subject having or at risk of developing a neoplasia (e.g., melanoma). The instructions will generally include information about the use of the composition for the treatment or prevention of a neoplasia (e.g., melanoma). In other embodiments, the instructions include at least one of the following: description of the therapeutic agent; dosage schedule and administration for treatment or prevention of a neoplasia (e.g., melanoma) or symptoms thereof; precautions; warnings; indications; counter-indications; overdosage information; adverse reactions; animal pharmacology; clinical studies; and/or references. The instructions may be printed directly on the container (when present), or as a label applied to the container, or as a separate sheet, pamphlet, card, or folder supplied in or with the container.

### EXAMPLES

The practice of the presently disclosed subject matter employs, unless otherwise indicated, conventional techniques of molecular biology (including recombinant techniques), microbiology, cell biology, biochemistry and immunology, which are well within the purview of the skilled artisan. Such techniques are explained fully in the literature, such as, “Molecular Cloning: A Laboratory Manual”, second edition (Sambrook, 1989); “Oligonucleotide Synthesis” (Gait, 1984); “Animal Cell Culture” (Freshney, 1987); “Methods in Enzymology” “Handbook of Experimental Immunology” (Weir, 1996); “Gene Transfer Vectors for Mammalian Cells” (Miller and Calos, 1987); “Current Protocols in Molecular Biology” (Ausubel, 1987); “PCR: The Polymerase Chain Reaction”, (Mullis, 1994); “Current Protocols in Immunology” (Coligan, 1991). These techniques are applicable to the production of the polynucleotides and polypeptides of the presently disclosed subject matter, and, as such, may be considered in making and practicing the presently disclosed subject matter. Particularly useful techniques for particular embodiments will be discussed in the sections that follow.

The following examples are put forth so as to provide those of ordinary skill in the art with a complete disclosure and description of how to make and use the compositions, and assay, screening, and therapeutic methods of the presently disclosed subject matter, and are not intended to limit the scope of what the inventors regard as the presently disclosed subject matter.

### Example 1: T Cells Expressing a CAR Targeting Melanoma Differentiation Antigens (MDA)

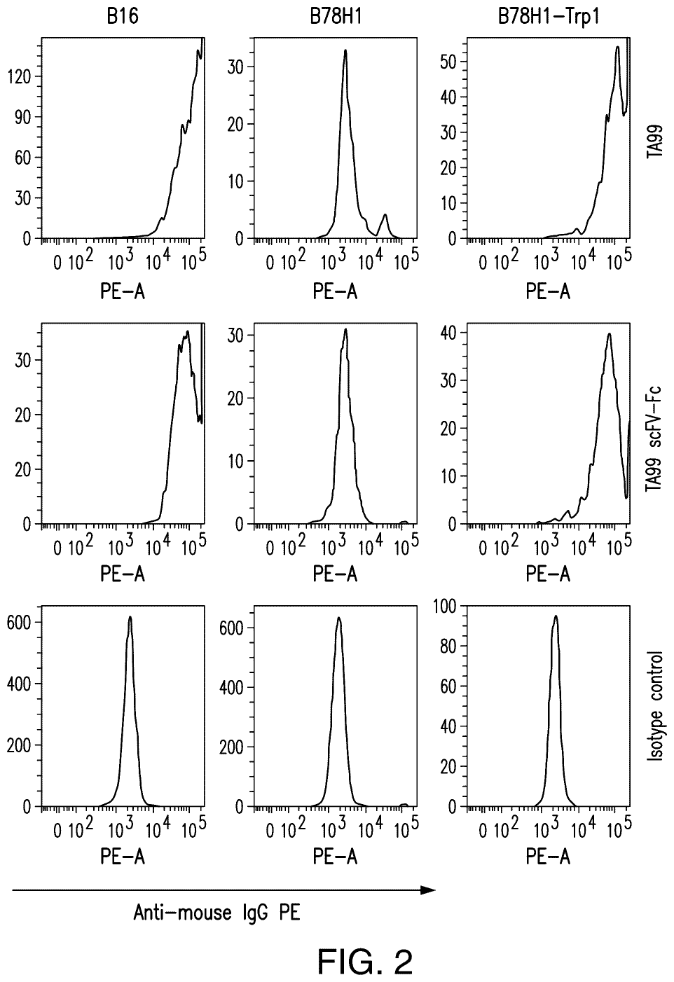

An scFv that specifically binds to a mouse Trp1 polypeptide and a human Trp1 polypeptide was generated from a murine monoclonal antibody TA99 that was generated from the TA99 antibody disclosed in International Patent Publication No. WO96/40249. This scFv was cloned into an eGFP cassette. The binding specificity of this scFv to the Trp1 polypeptide was evaluated using a soluble protein comprising this scFv and a Fc domain fused to the scFv. The binding specificity of the scFv-Fc fusion protein to B16 melanoma cells (expressing Trp1), a B16 variant B78H1 (not expressing Trp1), and B78H1-Trp1 (B78H1 engineered to express TRP1 on the surface) was evaluated by flow cytometry. As shown in FIG. 2, the scFv-Fc fusion portion showed comparable binding activity to the original TA99 antibody from which the scFv was derived. Once the binding specificity of the scFv was validated, a second generation CAR comprising this Trp1-specific scFv, a transmembrane domain comprising a CD8 polypeptide, an intracellular domain comprising a CD28 polypeptide, an intracellular domain comprising a CD3zeta polypeptide, and a co-stimulatory signaling region that comprises a CD28 polypeptide, was generated, which CAR is referred to as “TA99 CAR”, “TA99-CAR” or “CAR TA99”. Cloned T cell receptors (“TCRs”): the Trp1 TCRs and TCRs cloned from the OTII TCR transgenic mouse were used as positive controls. Plasmids were transfected into Plat-E retrovirus-producing cell line and after 10-15 days of geneticin selection, a clone derived from single cells was tested for retroviral expression by ELISA. Clones producing high titers of CAR retroviral vector were expanded and frozen.

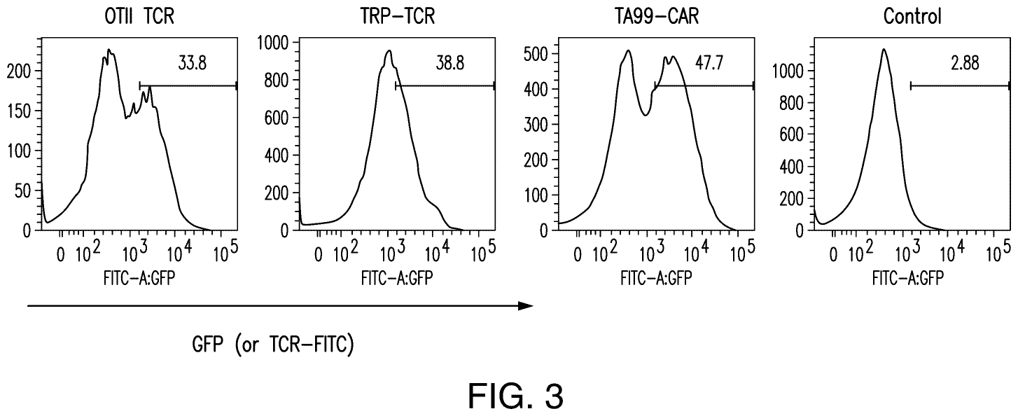

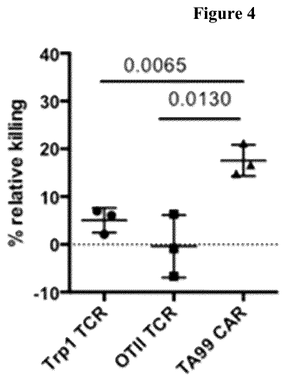

The functionality of the TA99 CAR was tested. Purified CD8⁺ T cells from naïve mice were stimulated with plate-bound anti-CD3/CD28 antibodies for 2 days in the presence of 30 U/ml of IL-2. In parallel, virus producing cells lines were grown to confluence. Supernatants from the virus producing cells were used to transduce activated CD8⁺ T cells with CAR- or TCR-expressing viruses as positive control. Transduction efficiency was tested by flow cytometry and was comparable to controls (FIG. 3). Subsequently, the TA99 CAR or control TCR transduced CD8⁺ T cells were tested for the ability to kill B16 melanoma cells in vitro. As shown in FIG. 4, the TA99 CAR transduced CD8⁺ T cells killed B16 cells more efficiently than Trp1 or OTII TCR transduced CD8⁺ T cells.

### Example 2: Explore Different Host Cell Types for CAR (CD8, CD4, γδ T, NK, NKT Cells) and Compare With Cells Bearing Trp1 TCR

Preparation and activation of cell types for retroviral transduction: CD8⁺, CD4⁺, γδ T and NK cells are purified by MACS from spleens and/or lymph nodes of naïve mice (3 mice as donors for CD8+ and CD4+ T cells, 15 mice per group as donors for γδ T and NK cells). CD8⁺ and CD4⁺ T cells are activated for 2 days with plate-bound anti-CD3/CD28 and 30 U/ml of IL-2. On day 2 after activation, T cells are transduced with high titer viral supernatants of either Trp1 TCR (or OTII as control) or the Trp1-targeted CAR of Example 1 (or Ova CAR as control) by a spin-infection method in the presence of protamine sulfate. The following day, T cells receive a second infection round. The activated T cells are rested for an additional 3-5 days in fresh media and the level of transduction tested by FACS. NK cells are activated with 1,000 U/ml of IL-2 and after 1-2 days in culture, are transduced as above. Three days after transduction, NK T cells are tested for expression of the Trp1-targeted CAR or Trp1 TCR. NK T cells are purified from liver by collagenase/DNA dissociation followed by a 33% Percol gradient (10 mice). The lymphocyte fraction is enriched for NK T cells by incubation with an α-GalCer/CD1d dimer-PE followed by anti PE-microbeads and MACS purification. NK T cells are expanded with 3 μg/ml of plate-bound anti-CD3, 100 U/ml IL-2, 1 ng/ml IL-12 and 1 μg/ml soluble anti-CD28. After 2-3 weeks, cells are transduced with the Trp1-targeted CAR or Trp1 TCR as above and expression of transgenes is tested. γδ T cells are cultured in the presence of 1 μg/ml of plate-bound anti-CD3 and 20 U/ml of IL-2. After 2 days in culture, activated γδ T cells are transduced with the Trp1-targeted CAR or Trp1 TCR as above and expression is tested 24 hours later. Only cells with transduction efficiencies >50% are used in subsequent assays.

B16 killing assays in vitro: To test the cytotoxic potential of the transduced cells in vitro, a collagen-fibrin clonogenic killing assay is performed. This assay has proven 5,000× more sensitive in detecting cytotoxicity than conventional killing assays. B16 cells (with/without effectors) are incubated in PBS containing 1 mg/ml fibrinogen, 1 mg/ml collagen I, 10% FBS, and 0.1 U thrombin. Gels are formed at 37° C.×20 min and 24 h later, lysed by sequential collagenase/trypsin digestion and recovered melanoma cells plated for colony formation. After a 7-day culture, plates are fixed, stained and colonies counted. 10:1 and 1:1 effector: target ratios are used. For preparation of targets, B16 cells are incubated overnight with 10 ng/ml IFN-γ and single cell suspensions are prepared. In addition to transfecting cells with the Trp1-targeted CAR of Example 1 or TCR directed against Ova as a control, 20 μg/ml of anti-MHC class II Ab or TA99 are also added to test that effector cells are killing in an antigen-dependent manner.

### Example 3: Testing Trp1-CAR T Cells In Vivo

T cells are transduced with the Trp1-CAR retrovirus and are transferred to established melanoma-bearing mice preconditioned with high dose cyclophosphamide or sub-lethal irradiation. Tumor size is periodically measured every 2-3 days. Once a curative regimen is established, purified Trp1-CAR CD4⁺ or CD8⁺ T cells are injected in separate experiment to measure the contribution of each subset to the therapeutic effects.

It has been shown that transfer of 1×10⁵ Trp1 TCR transduced CD4⁺ T cells can cure 20-40% of mice with established B16 tumors when mice are also given CTX. This suboptimal regression is ideal since improvement can be quantified. Therefore, mice bearing established tumors are injected with 250 mg/kg of CTX. The next day, 1×10⁵ CD8⁺ T, NK cells, NK T cells, γδ T cells and CD4⁺ T cells transduced with either Trp1-expressing or OTII-expressing TCRs, or Trp1-expressing or OVA-expressing CAR (15/group) are transferred to the mice and tumor growth is monitored. Optimal efficacy is defined as the largest number of animals with durable (>90 days) control of established tumors. To test that transduced cells kill B16 specifically and also assess for antigen spreading, the above process is repeated in mice injected with B16 in one flank and B78H1 in the other. B78H1 is a B16 variant that does not express Trp1.

### Example 4: Assessing the Bio-Distribution of Trp1-CAR T Cells in Vivo

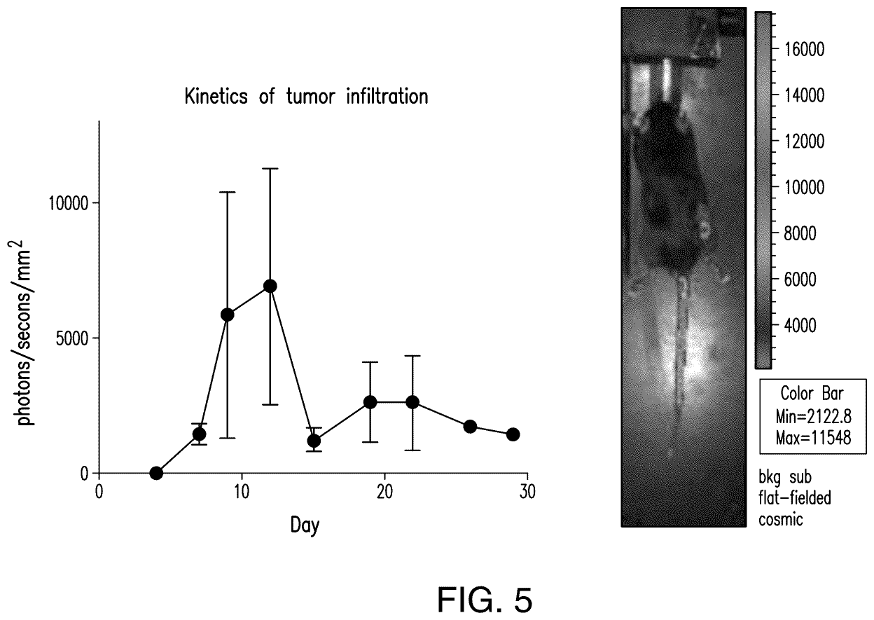

To measure the kinetics and distribution of the T cells transduced with the Trp1-CAR, an equivalent experiment is performed as above except donor T splenocytes and peripheral lymph nodes are isolated from transgenic mice expressing luciferase. Transduced luciferase-expressing cells are transferred to tumor-bearing mice pre-treated with CTX and whole animal images collected with an IVIS200 optical imaging system. Treated mice are imaged every 3-4 days by i.p. injection of D-luciferin (150 mg/kg) and 10 min exposure time. It is previously found that luciferase-Trp1 TCR CD4⁺ tumor infiltration peaks 14 days post-transfer, decaying to basal level at day 25-30 (FIG. 5). Kinetics of infiltration and persistence of the transferred cells are monitored (repeated ×3).

### Example 5: Combination of Trp1-CAR T Cells With Costimulatory Molecules or Checkpoint Blockade

Whether clinically relevant monoclonal antibodies known to potentiate T cell function can be combined with Trp1-CAR-expressing T cell to augment the anti-tumor properties is tested. Antibodies to be test in combination with Trp1-CAR T cells include: anti-CTLA-4, anti-PD1, anti-PD1L, anti-OX40, anti-GITR, anti-CD40, anti-4-1bb, anti-TIGT, anti-LAG3, anti-TNSF25. Optimally established doses of each antibody are administered. To optimize the schedule of each antibody, Trp1-CAR-expressing T cells are prepared as described above using congenic mice (such as CD45.1 or Thy1.1) donors. After adoptive transfer, the mice are sacrificed at several time-points and the expression of each target molecule (or its receptor) on Trp1-CAR-expressing T cells is tested ex-vivo by flow cytometry from single cell suspension prepared from tumors, draining lymph nodes, and spleens. After an adequate schedule is found for each molecule, a suboptimal dose of Trp1-CAR-expressing T cells or mice with more advanced tumor burden is administered to mice in combination with each of the monoclonal antibodies described. Tumors are periodically tested every 2-3 days until the conclusion of the experiment.

### Example 6: Testing Trp1-CAR NKT Cells In Vivo and In Vitro

NK T cell, being a versatile subset with both innate and adaptive like properties, can effectively remove tumors under certain circumstances. NK T cells are isolated from the spleens and livers of donors mice. After 48 hours of activation with anti-CD3/CD28 beads in low dose IL-2, NK T cells are transduced with high titer Trp1-CAR retrovirus. On day 7, the transduced NK T cells are incubated with irradiated B78H1 expressing surface Trp1. The ability of Trp1-CAR NK T cells to kill in vitro B16 melanoma or B78H1 expressing surface Trp1 is tested before adoptive transfer. After 7 days, the Trp1-CAR-expressing NK T cells are transferred to a B16 melanoma bearing mice preconditioned with high dose cyclophosphamide or sub-lethal irradiation. The tumors are periodically measured every 2-3 days.

### Example 7: Testing Trp1-CAR T Cells in Combination With Anti-Melanoma CD4+ or CD8+ T Cells

Trp1 expression by melanoma is both, intra- and extra-cellular. Therefore, the addition of T cells with TCRs against differentiation antigens can enhance the potency of the Trp1-CAR-expressing T cells. Trp1-CAR-expressing T cells are co-transferred with anti-melanoma CD4⁺ or CD8⁺ T cells. The CD4⁺ and CD8⁺ T melanoma-specific T cells are isolated TCR transgenic mice. A suboptimal dose of Trp1-CAR T cells or mice with a more advanced tumor burden are treated in combination with anti-melanoma CD4⁺ or CD8⁺ T cells as described above. The tumor size is measured periodically every 2-3 days.

### Example 8: Testing Efficacy and Persistance of Trp1-CAR T Cells In Vivo

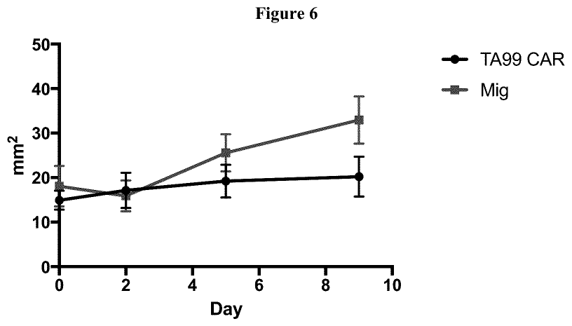

T cells were transduced with the TA99 CAR. The TA99 CAR-expressing T cells were transferred to mice (10 per group) bearing B16 melanoma tumors. The mice received cyclophosphamide at a dose of 250 mg/Kg before the CAR-T cell treatment. The next day, the mice were injected with either Mig (empty control vector) or the TA99 CAR transduced CD4⁺ and CD8⁺ T cells. Tumor size progression was measured over time and is shown in FIG. 6. Data points represent average size with standard error of the mean per group. Tumor progression in mice treated with the TA99 CAR T cells was significantly reduced compared with the control group.

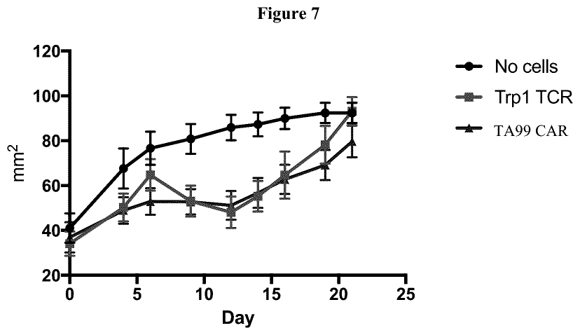

Mice (10 per group) bearing B16 melanoma were preconditioned with cyclophosphamide at a dose of 250 mg/Kg before the CAR T cell treatment. The next day, the mice were injected with CD4⁺ and CD8⁺ T cells transduced with either Trp1-TCR or the TA99 CAR. Tumor size progression was measured over time and is shown in FIG. 7. Data points represent average size with standard error of the mean per group. Tumor progression in mice treated with Trp1-TCR T cells was delayed compared to untreated mice. However, the efficacy of the Trp1-TCR T cell treatment diminished after 20 days of treatment, where average tumor size reached the same level with untreated mice. Tumor progression in mice treated with the TA99 CAR-expressing T cells was reduced compared to untreated mice, and improvement of efficacy over the Trp1-TCR T cell treatment was observed after 20 days of treatment, where average tumor size remained lower compared to untreated mice.

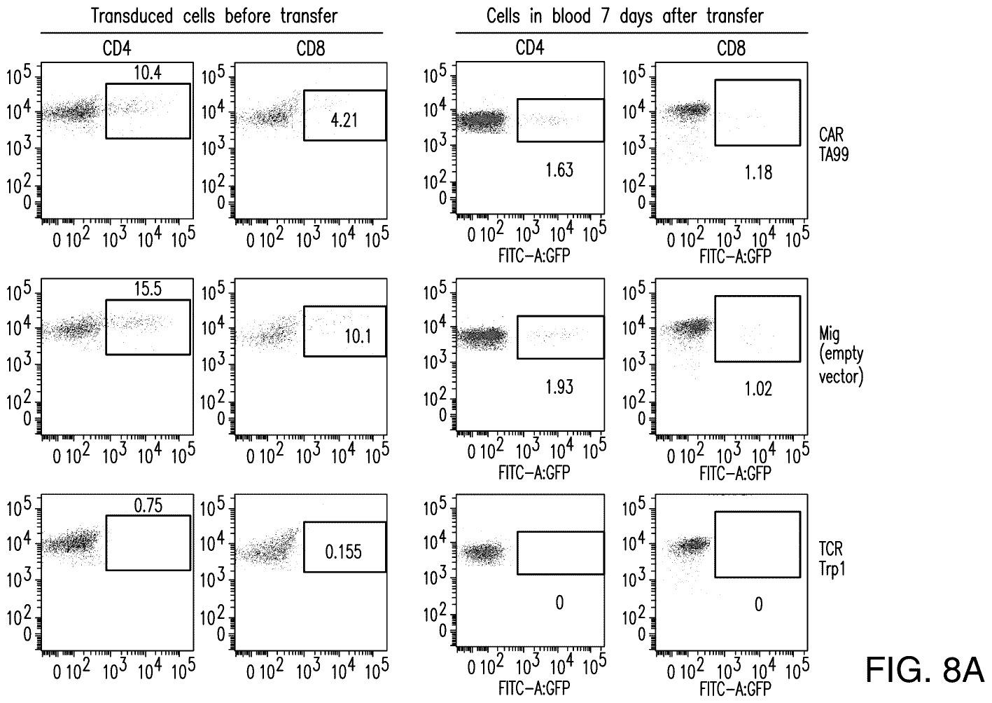

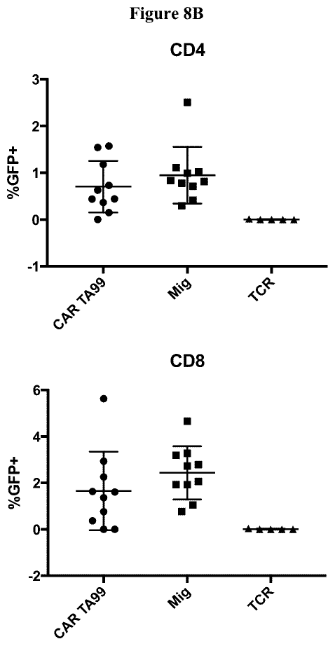

The persistence of the TA99 CAR-expressing T cells was also analyzed in vivo. Mice bearing B16 melanoma (9 to 10 per group) were given cyclophosphamide at a dose of 250 mg/Kg 21 days after tumor challenge. The next day, 100,000 CD4⁺ and CD8⁺ T cells transduced with the TA99 CAR or controls were administered through intravenous injection. After 7 days, the mice were bled retro-orbitally and lymphocytes in the blood were analyzed by flow cytometry before and after treatment. Events were gated on live CD4 and CD8. FIG. 8A shows one representative plot of the flow cytometry analyses. FIG. 8B shows the percentage of GFP (transduced with CARs or empty vector) 7 days after treatment. Cells transduced with the Trp1 TCR serve as positive control. The results indicate that a significant amount of the TA99 CAR-expressing T cells persisted in blood 7 days after T cell transfer.

From the foregoing description, it will be apparent that variations and modifications may be made to the presently disclosed subject matter described herein to adopt it to various usages and conditions. Such embodiments are also within the scope of the following claims.

All patents and publications and sequences referred to by accession or reference number mentioned in this specification are herein incorporated by reference to the same extent as if each independent patent and publication and sequence was specifically and individually indicated to be incorporated by reference.

1.-59. (canceled)

60. A method of reducing tumor burden in a subject and/or increasing or lengthening survival of a subject having neoplasia, comprising administering to the subject an effective amount of an immunoresponsive cell comprising an extracellular antigen-binding domain, a transmembrane domain, and an intracellular domain,
wherein the extracellular antigen-binding comprises a heavy chain variable region (VH) comprising a CDR1 comprising the amino acid sequence set forth in SEQ ID NO: 1, a CDR2 comprising the amino acid sequence set forth in SEQ ID NO: 2, and a CDR3 comprising the amino acid sequence set forth in SEQ ID NO: 3; and a light chain variable region (VL) comprising a CDR1 comprising the amino acid sequence set forth in SEQ ID NO: 4, a CDR2 comprising the amino acid sequence set forth in SEQ ID NO: 5, and a CDR3 comprising the amino acid sequence set forth in SEQ ID NO: 6,
thereby inducing tumor cell death in the subject and/or increasing or lengthening survival of the subject.

61. The method of claim 60, wherein the method reduces the number of tumor cells, reduces tumor size, and/or eradicates the tumor in the subject.

62.-63. (canceled)

64. The method of claim 60, wherein the tumor and/or neoplasia is associated with overexpression of one or more MDAs or Trp1.

65. (canceled)

66. The method of claim 60, wherein the tumor and/or neoplasia is selected from the group consisting of melanoma, glioblastoma multiforme, anaplastic astrocytoma, ependymoma, meningioma, oligodendroglioma, and combinations thereof.

67. The method of claim 66, wherein the tumor and/or neoplasia is melanoma.

68.-73. (canceled)

74. The method of claim 60, wherein the subject is a human.

75. The method of claim 60, wherein the immunoresponsive cell is a T cell.

76. The method of claim 60, further comprising administering to the subject a chemotherapeutic agent.

77. The method of claim 76, wherein the chemotherapeutic agent is selected from the group consisting of docetaxel, cyclophosphamide, capecitabine, doxorubic, and fludarabin.

78. The method of claim 77, wherein the chemotherapeutic agent is cyclophosphamide.

79.-85. (canceled)

86. The method of claim 60, wherein the V_{H} comprises the amino acid sequence set forth in SEQ ID NO:7, and the V_{L} comprises the amino acid sequence set forth in SEQ ID NO: 8.

87. The method of claim 60, wherein the extracellular antigen-binding domain a single-chain variable fragment (scFv), a Fab, or a F(ab)₂.

88. The method of claim 60, wherein the transmembrane domain comprises a CD8 polypeptide, a CD28 polypeptide, a CD3zeta polypeptide, a CD4 polypeptide, a 4-1BB polypeptide, an OX40 polypeptide, an ICOS polypeptide, a CTLA-4 polypeptide, a PD-1 polypeptide, a LAG-3 polypeptide, a 2B4 polypeptide, a BTLA polypeptide, a synthetic peptide (not based on a protein associated with the immune response), or a combination thereof.

89. The method of claim 88, wherein the transmembrane domain comprises a CD8 polypeptide.

90. The method of claim 60, wherein the intracellular domain comprises a CD3zeta polypeptide.

91. The method of claim 60, wherein the intracellular domain further comprises at least one co-stimulatory signaling region.

92. The method of claim 91, the at least one co-stimulatory signaling region comprises a CD28 polypeptide, a 4-1BB polypeptide, an OX40 polypeptide, an ICOS polypeptide, a DAP-10 polypeptide, or a combination thereof.

93. The method of claim 92, the at least one co-stimulatory signaling region comprises a CD28 polypeptide.

94. The method of claim 60, wherein the transmembrane domain comprises a CD8 polypeptide, and the intracellular domain that comprises a CD3zeta polypeptide and a co-stimulatory signaling region that comprises a CD28 polypeptide.

95. The method of claim 75, wherein the T cell is selected from the group consisting of a cytotoxic T lymphocyte (CTL), a regulatory T cell, an NK T cell, and a central memory T cell.
